SEQUENCE LISTING (294 sequences)

SEQ ID NO: 1 - DNA, 25 bp, synthetic construct
  misc_feature 1..25: note = Description of Artificial Sequence: Syntheticprobe
  source 1..25: mol_type = other DNA
ataggtgattttggtctagctacag

SEQ ID NO: 2 - DNA, 22 bp, synthetic construct
  misc_feature 1..22: note = Description of Artificial Sequence: Syntheticprobe
  source 1..22: mol_type = other DNA
tgaaatctcgatggagtgggtc

SEQ ID NO: 3 - DNA, 22 bp, synthetic construct
  misc_feature 1..22: note = Description of Artificial Sequence: Syntheticprobe
  source 1..22: mol_type = other DNA
agaaatctcgatggagtgggtc

SEQ ID NO: 4 - DNA, 23 bp, synthetic construct
  misc_feature 1..23: note = Description of Artificial Sequence: Syntheticprobe
  source 1..23: mol_type = other DNA
tgtcaagatcacagattttgggc

SEQ ID NO: 5 - DNA, 16 bp, synthetic construct
  misc_feature 1..16: note = Description of Artificial Sequence: Syntheticprobe
  source 1..16: mol_type = other DNA
tggccaaactgctggg

SEQ ID NO: 6 - DNA, 16 bp, synthetic construct
  misc_feature 1..16: note = Description of Artificial Sequence: Syntheticprobe
  source 1..16: mol_type = other DNA
gggccaaactgctggg

SEQ ID NO: 7 - DNA, 17 bp, synthetic construct
  misc_feature 1..17: note = Description of Artificial Sequence: Syntheticprobe
  source 1..17: mol_type = other DNA
caccgtgcagctcatca

SEQ ID NO: 8 - DNA, 17 bp, synthetic construct
  misc_feature 1..17: note = Description of Artificial Sequence: Syntheticprobe
  source 1..17: mol_type = other DNA
cgcagctcatgcccttc

SEQ ID NO: 9 - DNA, 17 bp, synthetic construct
  misc_feature 1..17: note = Description of Artificial Sequence: Syntheticprobe
  source 1..17: mol_type = other DNA
tgcagctcatgcccttc

SEQ ID NO: 10 - DNA, 26 bp, synthetic construct
  misc_feature 1..26: note = Description of Artificial Sequence: Syntheticprobe
  source 1..26: mol_type = other DNA
ataggtgattttggtctagctacagt

SEQ ID NO: 11 - DNA, 26 bp, synthetic construct
  misc_feature 1..26: note = Description of Artificial Sequence: Syntheticprobe
  source 1..26: mol_type = other DNA
ataggtgattttggtctagctacaga

SEQ ID NO: 12 - DNA, 21 bp, synthetic construct
  misc_feature 1..21: note = Description of Artificial Sequence: Syntheticprobe
  source 1..21: mol_type = other DNA
gaaatctcgatggagtgggtc

SEQ ID NO: 13 - DNA, 24 bp, synthetic construct
  misc_feature 1..24: note = Description of Artificial Sequence: Syntheticprobe
  source 1..24: mol_type = other DNA
tgtcaagatcacagattttgggct

SEQ ID NO: 14 - DNA, 24 bp, synthetic construct
  misc_feature 1..24: note = Description of Artificial Sequence: Syntheticprobe
  source 1..24: mol_type = other DNA
tgtcaagatcacagattttgggcg

SEQ ID NO: 15 - DNA, 16 bp, synthetic construct
  misc_feature 1..16: note = Description of Artificial Sequence: Syntheticprobe
  source 1..16: mol_type = other DNA
ggccaaactgctgggt

SEQ ID NO: 16 - DNA, 18 bp, synthetic construct
  misc_feature 1..18: note = Description of Artificial Sequence: Syntheticprobe
  source 1..18: mol_type = other DNA
caccgtgcagctcatcac

SEQ ID NO: 17 - DNA, 18 bp, synthetic construct
  misc_feature 1..18: note = Description of Artificial Sequence: Syntheticprobe
  source 1..18: mol_type = other DNA
caccgtgcagctcatcat

SEQ ID NO: 18 - DNA, 17 bp, synthetic construct
  misc_feature 1..17: note = Description of Artificial Sequence: Syntheticprobe
  source 1..17: mol_type = other DNA
gcagctcatgcccttcg

SEQ ID NO: 19 - DNA, 25 bp, synthetic construct
  misc_feature 1..25: note = Description of Artificial Sequence: Syntheticprobe
  source 1..25: mol_type = other DNA
cagattttgggctggccaaactgct

SEQ ID NO: 20 - DNA, 25 bp, synthetic construct
  misc_feature 1..25: note = Description of Artificial Sequence: Syntheticprobe
  source 1..25: mol_type = other DNA
cagattttgggagggccaaactgct

SEQ ID NO: 21 - DNA, 25 bp, synthetic construct
  misc_feature 1..25: note = Description of Artificial Sequence: Syntheticprobe
  source 1..25: mol_type = other DNA
agattttgggctggccaaactgctg

SEQ ID NO: 22 - DNA, 25 bp, synthetic construct
  misc_feature 1..25: note = Description of Artificial Sequence: Syntheticprobe
  source 1..25: mol_type = other DNA
agattttgggagggccaaactgctg

SEQ ID NO: 23 - DNA, 24 bp, synthetic construct
  misc_feature 1..24: note = Description of Artificial Sequence: Syntheticprobe
  source 1..24: mol_type = other DNA
cgtgcagctcatcacgcagctcat

SEQ ID NO: 24 - DNA, 24 bp, synthetic construct
  misc_feature 1..24: note = Description of Artificial Sequence: Syntheticprobe
  source 1..24: mol_type = other DNA
cgtgcagctcatcatgcagctcat

SEQ ID NO: 25 - DNA, 25 bp, synthetic construct
  misc_feature 1..25: note = Description of Artificial Sequence: Syntheticprobe
  source 1..25: mol_type = other DNA
ccgtgcagctcatcacgcagctcat

SEQ ID NO: 26 - DNA, 25 bp, synthetic construct
  misc_feature 1..25: note = Description of Artificial Sequence: Syntheticprobe
  source 1..25: mol_type = other DNA
ccgtgcagctcatcatgcagctcat

SEQ ID NO: 27 - DNA, 26 bp, synthetic construct
  misc_feature 1..26: note = Description of Artificial Sequence: Syntheticprobe
  source 1..26: mol_type = other DNA
accgtgcagctcatcacgcagctcat

SEQ ID NO: 28 - DNA, 26 bp, synthetic construct
  misc_feature 1..26: note = Description of Artificial Sequence: Syntheticprobe
  source 1..26: mol_type = other DNA
accgtgcagctcatcatgcagctcat

SEQ ID NO: 29 - DNA, 26 bp, synthetic construct
  misc_feature 1..26: note = Description of Artificial Sequence: Syntheticprobe
  source 1..26: mol_type = other DNA
cgtgcagctcatcacgcagctcatgc

SEQ ID NO: 30 - DNA, 26 bp, synthetic construct
  misc_feature 1..26: note = Description of Artificial Sequence: Syntheticprobe
  source 1..26: mol_type = other DNA
cgtgcagctcatcatgcagctcatgc

SEQ ID NO: 31 - DNA, 26 bp, synthetic construct
  misc_feature 1..26: note = Description of Artificial Sequence: Syntheticprobe
  source 1..26: mol_type = other DNA
gcagctcatcacgcagctcatgccct

SEQ ID NO: 32 - DNA, 26 bp, synthetic construct
  misc_feature 1..26: note = Description of Artificial Sequence: Syntheticprobe
  source 1..26: mol_type = other DNA
gcagctcatcatgcagctcatgccct

SEQ ID NO: 33 - DNA, 26 bp, synthetic construct
  misc_feature 1..26: note = Description of Artificial Sequence: Syntheticprobe
  source 1..26: mol_type = other DNA
cagctcatcacgcagctcatgccctt

SEQ ID NO: 34 - DNA, 26 bp, synthetic construct
  misc_feature 1..26: note = Description of Artificial Sequence: Syntheticprobe
  source 1..26: mol_type = other DNA
cagctcatcatgcagctcatgccctt

SEQ ID NO: 35 - DNA, 27 bp, synthetic construct
  misc_feature 1..27: note = Description of Artificial Sequence: Syntheticprobe
  source 1..27: mol_type = other DNA
ccgtgcagctcatcacgcagctcatgc

SEQ ID NO: 36 - DNA, 27 bp, synthetic construct
  misc_feature 1..27: note = Description of Artificial Sequence: Syntheticprobe
  source 1..27: mol_type = other DNA
ccgtgcagctcatcatgcagctcatgc

SEQ ID NO: 37 - DNA, 27 bp, synthetic construct
  misc_feature 1..27: note = Description of Artificial Sequence: Syntheticprobe
  source 1..27: mol_type = other DNA
cgtgcagctcatcacgcagctcatgcc

SEQ ID NO: 38 - DNA, 27 bp, synthetic construct
  misc_feature 1..27: note = Description of Artificial Sequence: Syntheticprobe
  source 1..27: mol_type = other DNA
cgtgcagctcatcatgcagctcatgcc

SEQ ID NO: 39 - DNA, 27 bp, synthetic construct
  misc_feature 1..27: note = Description of Artificial Sequence: Syntheticprobe
  source 1..27: mol_type = other DNA
gtgcagctcatcacgcagctcatgccc

SEQ ID NO: 40 - DNA, 27 bp, synthetic construct
  misc_feature 1..27: note = Description of Artificial Sequence: Syntheticprobe
  source 1..27: mol_type = other DNA
gtgcagctcatcatgcagctcatgccc

SEQ ID NO: 41 - DNA, 27 bp, synthetic construct
  misc_feature 1..27: note = Description of Artificial Sequence: Syntheticprobe
  source 1..27: mol_type = other DNA
tgcagctcatcacgcagctcatgccct

SEQ ID NO: 42 - DNA, 27 bp, synthetic construct
  misc_feature 1..27: note = Description of Artificial Sequence: Syntheticprobe
  source 1..27: mol_type = other DNA
tgcagctcatcatgcagctcatgccct

SEQ ID NO: 43 - DNA, 27 bp, synthetic construct
  misc_feature 1..27: note = Description of Artificial Sequence: Syntheticprobe
  source 1..27: mol_type = other DNA
gcagctcatcacgcagctcatgccctt

SEQ ID NO: 44 - DNA, 27 bp, synthetic construct
  misc_feature 1..27: note = Description of Artificial Sequence: Syntheticprobe
  source 1..27: mol_type = other DNA
gcagctcatcatgcagctcatgccctt

SEQ ID NO: 45 - DNA, 27 bp, synthetic construct
  misc_feature 1..27: note = Description of Artificial Sequence: Syntheticprobe
  source 1..27: mol_type = other DNA
cagctcatcacgcagctcatgcccttc

SEQ ID NO: 46 - DNA, 27 bp, synthetic construct
  misc_feature 1..27: note = Description of Artificial Sequence: Syntheticprobe
  source 1..27: mol_type = other DNA
cagctcatcatgcagctcatgcccttc

SEQ ID NO: 47 - DNA, 28 bp, synthetic construct
  misc_feature 1..28: note = Description of Artificial Sequence: Syntheticprobe
  source 1..28: mol_type = other DNA
accgtgcagctcatcacgcagctcatgc

SEQ ID NO: 48 - DNA, 28 bp, synthetic construct
  misc_feature 1..28: note = Description of Artificial Sequence: Syntheticprobe
  source 1..28: mol_type = other DNA
accgtgcagctcatcatgcagctcatgc

SEQ ID NO: 49 - DNA, 28 bp, synthetic construct
  misc_feature 1..28: note = Description of Artificial Sequence: Syntheticprobe
  source 1..28: mol_type = other DNA
ccgtgcagctcatcacgcagctcatgcc

SEQ ID NO: 50 - DNA, 28 bp, synthetic construct
  misc_feature 1..28: note = Description of Artificial Sequence: Syntheticprobe
  source 1..28: mol_type = other DNA
ccgtgcagctcatcatgcagctcatgcc

SEQ ID NO: 51 - DNA, 28 bp, synthetic construct
  misc_feature 1..28: note = Description of Artificial Sequence: Syntheticprobe
  source 1..28: mol_type = other DNA
cgtgcagctcatcacgcagctcatgccc

SEQ ID NO: 52 - DNA, 28 bp, synthetic construct
  misc_feature 1..28: note = Description of Artificial Sequence: Syntheticprobe
  source 1..28: mol_type = other DNA
cgtgcagctcatcatgcagctcatgccc

SEQ ID NO: 53 - DNA, 28 bp, synthetic construct
  misc_feature 1..28: note = Description of Artificial Sequence: Syntheticprobe
  source 1..28: mol_type = other DNA
gtgcagctcatcacgcagctcatgccct

SEQ ID NO: 54 - DNA, 28 bp, synthetic construct
  misc_feature 1..28: note = Description of Artificial Sequence: Syntheticprobe
  source 1..28: mol_type = other DNA
gtgcagctcatcatgcagctcatgccct

SEQ ID NO: 55 - DNA, 28 bp, synthetic construct
  misc_feature 1..28: note = Description of Artificial Sequence: Syntheticprobe
  source 1..28: mol_type = other DNA
tgcagctcatcacgcagctcatgccctt

SEQ ID NO: 56 - DNA, 28 bp, synthetic construct
  misc_feature 1..28: note = Description of Artificial Sequence: Syntheticprobe
  source 1..28: mol_type = other DNA
tgcagctcatcatgcagctcatgccctt

SEQ ID NO: 57 - DNA, 28 bp, synthetic construct
  misc_feature 1..28: note = Description of Artificial Sequence: Syntheticprobe
  source 1..28: mol_type = other DNA
gcagctcatcacgcagctcatgcccttc

SEQ ID NO: 58 - DNA, 28 bp, synthetic construct
  misc_feature 1..28: note = Description of Artificial Sequence: Syntheticprobe
  source 1..28: mol_type = other DNA
gcagctcatcatgcagctcatgcccttc

SEQ ID NO: 59 - DNA, 29 bp, synthetic construct
  misc_feature 1..29: note = Description of Artificial Sequence: Syntheticprobe
  source 1..29: mol_type = other DNA
caccgtgcagctcatcacgcagctcatgc

SEQ ID NO: 60 - DNA, 29 bp, synthetic construct
  misc_feature 1..29: note = Description of Artificial Sequence: Syntheticprobe
  source 1..29: mol_type = other DNA
caccgtgcagctcatcatgcagctcatgc

SEQ ID NO: 61 - DNA, 29 bp, synthetic construct
  misc_feature 1..29: note = Description of Artificial Sequence: Syntheticprobe
  source 1..29: mol_type = other DNA
accgtgcagctcatcacgcagctcatgcc

SEQ ID NO: 62 - DNA, 29 bp, synthetic construct
  misc_feature 1..29: note = Description of Artificial Sequence: Syntheticprobe
  source 1..29: mol_type = other DNA
accgtgcagctcatcatgcagctcatgcc

SEQ ID NO: 63 - DNA, 29 bp, synthetic construct
  misc_feature 1..29: note = Description of Artificial Sequence: Syntheticprobe
  source 1..29: mol_type = other DNA
ccgtgcagctcatcacgcagctcatgccc

SEQ ID NO: 64 - DNA, 29 bp, synthetic construct
  misc_feature 1..29: note = Description of Artificial Sequence: Syntheticprobe
  source 1..29: mol_type = other DNA
ccgtgcagctcatcatgcagctcatgccc

SEQ ID NO: 65 - DNA, 29 bp, synthetic construct
  misc_feature 1..29: note = Description of Artificial Sequence: Syntheticprobe
  source 1..29: mol_type = other DNA
cgtgcagctcatcacgcagctcatgccct

SEQ ID NO: 66 - DNA, 29 bp, synthetic construct
  misc_feature 1..29: note = Description of Artificial Sequence: Syntheticprobe
  source 1..29: mol_type = other DNA
cgtgcagctcatcatgcagctcatgccct

SEQ ID NO: 67 - DNA, 29 bp, synthetic construct
  misc_feature 1..29: note = Description of Artificial Sequence: Syntheticprobe
  source 1..29: mol_type = other DNA
gtgcagctcatcacgcagctcatgccctt

SEQ ID NO: 68 - DNA, 29 bp, synthetic construct
  misc_feature 1..29: note = Description of Artificial Sequence: Syntheticprobe
  source 1..29: mol_type = other DNA
gtgcagctcatcatgcagctcatgccctt

SEQ ID NO: 69 - DNA, 29 bp, synthetic construct
  misc_feature 1..29: note = Description of Artificial Sequence: Syntheticprobe
  source 1..29: mol_type = other DNA
tgcagctcatcacgcagctcatgcccttc

SEQ ID NO: 70 - DNA, 29 bp, synthetic construct
  misc_feature 1..29: note = Description of Artificial Sequence: Syntheticprobe
  source 1..29: mol_type = other DNA
tgcagctcatcatgcagctcatgcccttc

SEQ ID NO: 71 - DNA, 30 bp, synthetic construct
  misc_feature 1..30: note = Description of Artificial Sequence: Syntheticprobe
  source 1..30: mol_type = other DNA
ccaccgtgcagctcatcacgcagctcatgc

SEQ ID NO: 72 - DNA, 30 bp, synthetic construct
  misc_feature 1..30: note = Description of Artificial Sequence: Syntheticprobe
  source 1..30: mol_type = other DNA
ccaccgtgcagctcatcatgcagctcatgc

SEQ ID NO: 73 - DNA, 30 bp, synthetic construct
  misc_feature 1..30: note = Description of Artificial Sequence: Syntheticprobe
  source 1..30: mol_type = other DNA
caccgtgcagctcatcacgcagctcatgcc

SEQ ID NO: 74 - DNA, 30 bp, synthetic construct
  misc_feature 1..30: note = Description of Artificial Sequence: Syntheticprobe
  source 1..30: mol_type = other DNA
caccgtgcagctcatcatgcagctcatgcc

SEQ ID NO: 75 - DNA, 30 bp, synthetic construct
  misc_feature 1..30: note = Description of Artificial Sequence: Syntheticprobe
  source 1..30: mol_type = other DNA
accgtgcagctcatcacgcagctcatgccc

SEQ ID NO: 76 - DNA, 30 bp, synthetic construct
  misc_feature 1..30: note = Description of Artificial Sequence: Syntheticprobe
  source 1..30: mol_type = other DNA
accgtgcagctcatcatgcagctcatgccc

SEQ ID NO: 77 - DNA, 30 bp, synthetic construct
  misc_feature 1..30: note = Description of Artificial Sequence: Syntheticprobe
  source 1..30: mol_type = other DNA
ccgtgcagctcatcacgcagctcatgccct

SEQ ID NO: 78 - DNA, 30 bp, synthetic construct
  misc_feature 1..30: note = Description of Artificial Sequence: Syntheticprobe
  source 1..30: mol_type = other DNA
ccgtgcagctcatcatgcagctcatgccct

SEQ ID NO: 79 - DNA, 30 bp, synthetic construct
  misc_feature 1..30: note = Description of Artificial Sequence: Syntheticprobe
  source 1..30: mol_type = other DNA
cgtgcagctcatcacgcagctcatgccctt

SEQ ID NO: 80 - DNA, 30 bp, synthetic construct
  misc_feature 1..30: note = Description of Artificial Sequence: Syntheticprobe
  source 1..30: mol_type = other DNA
cgtgcagctcatcatgcagctcatgccctt

SEQ ID NO: 81 - DNA, 30 bp, synthetic construct
  misc_feature 1..30: note = Description of Artificial Sequence: Syntheticprobe
  source 1..30: mol_type = other DNA
gtgcagctcatcacgcagctcatgcccttc

SEQ ID NO: 82 - DNA, 30 bp, synthetic construct
  misc_feature 1..30: note = Description of Artificial Sequence: Syntheticprobe
  source 1..30: mol_type = other DNA
gtgcagctcatcatgcagctcatgcccttc

SEQ ID NO: 83 - DNA, 24 bp, synthetic construct
  misc_feature 1..24: note = Description of Artificial Sequence: Syntheticprobe
  source 1..24: mol_type = other DNA
gattttgggctggccaaactgctg

SEQ ID NO: 84 - DNA, 24 bp, synthetic construct
  misc_feature 1..24: note = Description of Artificial Sequence: Syntheticprobe
  source 1..24: mol_type = other DNA
gattttgggcgagccaaactgctg

SEQ ID NO: 85 - DNA, 25 bp, synthetic construct
  misc_feature 1..25: note = Description of Artificial Sequence: Syntheticprobe
  source 1..25: mol_type = other DNA
cagattttgggctggccaaactgct

SEQ ID NO: 86 - DNA, 25 bp, synthetic construct
  misc_feature 1..25: note = Description of Artificial Sequence: Syntheticprobe
  source 1..25: mol_type = other DNA
cagattttgggcgagccaaactgct

SEQ ID NO: 87 - DNA, 25 bp, synthetic construct
  misc_feature 1..25: note = Description of Artificial Sequence: Syntheticprobe
  source 1..25: mol_type = other DNA
agattttgggctggccaaactgctg

SEQ ID NO: 88 - DNA, 25 bp, synthetic construct
  misc_feature 1..25: note = Description of Artificial Sequence: Syntheticprobe
  source 1..25: mol_type = other DNA
agattttgggcgagccaaactgctg

SEQ ID NO: 89 - DNA, 24 bp, synthetic construct
  misc_feature 1..24: note = Description of Artificial Sequence: Syntheticprobe
  source 1..24: mol_type = other DNA
gattttgggctggccaaactgctg

SEQ ID NO: 90 - DNA, 24 bp, synthetic construct
  misc_feature 1..24: note = Description of Artificial Sequence: Syntheticprobe
  source 1..24: mol_type = other DNA
gattttgggcgtgccaaactgctg

SEQ ID NO: 91 - DNA, 22 bp, synthetic construct
  misc_feature 1..22: note = Description of Artificial Sequence: Syntheticprobe
  source 1..22: mol_type = other DNA
gattttgggctggccaaactgc

SEQ ID NO: 92 - DNA, 22 bp, synthetic construct
  misc_feature 1..22: note = Description of Artificial Sequence: Syntheticprobe
  source 1..22: mol_type = other DNA
gattttgggcgggccaaactgc

SEQ ID NO: 93 - DNA, 29 bp, synthetic construct
  misc_feature 1..29: note = Description of Artificial Sequence: Syntheticprobe
  source 1..29: mol_type = other DNA
cttgcctacgccaccagctccaactacca

SEQ ID NO: 94 - DNA, 29 bp, synthetic construct
  misc_feature 1..29: note = Description of Artificial Sequence: Syntheticprobe
  source 1..29: mol_type = other DNA
cttgcctacgccatcagctccaactacca

SEQ ID NO: 95 - DNA, 30 bp, synthetic construct
  misc_feature 1..30: note = Description of Artificial Sequence: Syntheticprobe
  source 1..30: mol_type = other DNA
gcactcttgcctacgccaccagctccaact

SEQ ID NO: 96 - DNA, 30 bp, synthetic construct
  misc_feature 1..30: note = Description of Artificial Sequence: Syntheticprobe
  source 1..30: mol_type = other DNA
gcactcttgcctacgccatcagctccaact

SEQ ID NO: 97 - DNA, 30 bp, synthetic construct
  misc_feature 1..30: note = Description of Artificial Sequence: Syntheticprobe
  source 1..30: mol_type = other DNA
tcttgcctacgccaccagctccaactacca

SEQ ID NO: 98 - DNA, 30 bp, synthetic construct
  misc_feature 1..30: note = Description of Artificial Sequence: Syntheticprobe
  source 1..30: mol_type = other DNA
tcttgcctacgccatcagctccaactacca

SEQ ID NO: 99 - DNA, 30 bp, synthetic construct
  misc_feature 1..30: note = Description of Artificial Sequence: Syntheticprobe
  source 1..30: mol_type = other DNA
cttgcctacgccaccagctccaactaccac

SEQ ID NO: 100 - DNA, 30 bp, synthetic construct
  misc_feature 1..30: note = Description of Artificial Sequence: Syntheticprobe
  source 1..30: mol_type = other DNA
cttgcctacgccatcagctccaactaccac

SEQ ID NO: 101 - DNA, 29 bp, synthetic construct
  misc_feature 1..29: note = Description of Artificial Sequence: Syntheticprobe
  source 1..29: mol_type = other DNA
cttgcctacgccaccagctccaactacca

SEQ ID NO: 102 - DNA, 29 bp, synthetic construct
  misc_feature 1..29: note = Description of Artificial Sequence: Syntheticprobe
  source 1..29: mol_type = other DNA
cttgcctacgccagcagctccaactacca

SEQ ID NO: 103 - DNA, 30 bp, synthetic construct; identical to SEQ ID NO: 95
  misc_feature 1..30: note = Description of Artificial Sequence: Syntheticprobe
  source 1..30: mol_type = other DNA

SEQ ID NO: 104 - DNA, 30 bp, synthetic construct
  misc_feature 1..30: note = Description of Artificial Sequence: Syntheticprobe
  source 1..30: mol_type = other DNA
gcactcttgcctacgccagcagctccaact

SEQ ID NO: 105 - DNA, 30 bp, synthetic construct; identical to SEQ ID NO: 97
  misc_feature 1..30: note = Description of Artificial Sequence: Syntheticprobe
  source 1..30: mol_type = other DNA

SEQ ID NO: 106 - DNA, 30 bp, synthetic construct
  misc_feature 1..30: note = Description of Artificial Sequence: Syntheticprobe
  source 1..30: mol_type = other DNA
tcttgcctacgccagcagctccaactacca

SEQ ID NO: 107 - DNA, 30 bp, synthetic construct; identical to SEQ ID NO: 99
  misc_feature 1..30: note = Description of Artificial Sequence: Syntheticprobe
  source 1..30: mol_type = other DNA

SEQ ID NO: 108 - DNA, 30 bp, synthetic construct
  misc_feature 1..30: note = Description of Artificial Sequence: Syntheticprobe
  source 1..30: mol_type = other DNA
cttgcctacgccagcagctccaactaccac

SEQ ID NO: 109 - DNA, 29 bp, synthetic construct
  misc_feature 1..29: note = Description of Artificial Sequence: Syntheticprobe
  source 1..29: mol_type = other DNA
cttgcctacgccaccagctccaactacca

SEQ ID NO: 110 - DNA, 29 bp, synthetic construct
  misc_feature 1..29: note = Description of Artificial Sequence: Syntheticprobe
  source 1..29: mol_type = other DNA
cttgcctacgccgcaagctccaactacca

SEQ ID NO: 111 - DNA, 30 bp, synthetic construct; identical to SEQ ID NO: 95
  misc_feature 1..30: note = Description of Artificial Sequence: Syntheticprobe
  source 1..30: mol_type = other DNA

SEQ ID NO: 112 - DNA, 30 bp, synthetic construct
  misc_feature 1..30: note = Description of Artificial Sequence: Syntheticprobe
  source 1..30: mol_type = other DNA
gcactcttgcctacgccgcaagctccaact

SEQ ID NO: 113 - DNA, 30 bp, synthetic construct; identical to SEQ ID NO: 97
  misc_feature 1..30: note = Description of Artificial Sequence: Syntheticprobe
  source 1..30: mol_type = other DNA

SEQ ID NO: 114 - DNA, 30 bp, synthetic construct
  misc_feature 1..30: note = Description of Artificial Sequence: Syntheticprobe
  source 1..30: mol_type = other DNA
tcttgcctacgccgcaagctccaactacca

SEQ ID NO: 115 - DNA, 30 bp, synthetic construct; identical to SEQ ID NO: 99
  misc_feature 1..30: note = Description of Artificial Sequence: Syntheticprobe
  source 1..30: mol_type = other DNA

SEQ ID NO: 116 - DNA, 30 bp, synthetic construct
  misc_feature 1..30: note = Description of Artificial Sequence: Syntheticprobe
  source 1..30: mol_type = other DNA
cttgcctacgccgcaagctccaactaccac

SEQ ID NO: 117 - DNA, 29 bp, synthetic construct
  misc_feature 1..29: note = Description of Artificial Sequence: Syntheticprobe
  source 1..29: mol_type = other DNA
cttgcctacgccaccagctccaactacca

SEQ ID NO: 118 - DNA, 29 bp, synthetic construct
  misc_feature 1..29: note = Description of Artificial Sequence: Syntheticprobe
  source 1..29: mol_type = other DNA
cttgcctacgccgtcagctccaactacca

SEQ ID NO: 119 - DNA, 30 bp, synthetic construct; identical to SEQ ID NO: 95
  misc_feature 1..30: note = Description of Artificial Sequence: Syntheticprobe
  source 1..30: mol_type = other DNA

SEQ ID NO: 120 - DNA, 30 bp, synthetic construct
  misc_feature 1..30: note = Description of Artificial Sequence: Syntheticprobe
  source 1..30: mol_type = other DNA
gcactcttgcctacgccgtcagctccaact

SEQ ID NO: 121 - DNA, 30 bp, synthetic construct; identical to SEQ ID NO: 97
  misc_feature 1..30: note = Description of Artificial Sequence: Syntheticprobe
  source 1..30: mol_type = other DNA

SEQ ID NO: 122 - DNA, 30 bp, synthetic construct
  misc_feature 1..30: note = Description of Artificial Sequence: Syntheticprobe
  source 1..30: mol_type = other DNA
tcttgcctacgccgtcagctccaactacca

SEQ ID NO: 123 - DNA, 30 bp, synthetic construct; identical to SEQ ID NO: 99
  misc_feature 1..30: note = Description of Artificial Sequence: Syntheticprobe
  source 1..30: mol_type = other DNA

SEQ ID NO: 124 - DNA, 30 bp, synthetic construct
  misc_feature 1..30: note = Description of Artificial Sequence: Syntheticprobe
  source 1..30: mol_type = other DNA
cttgcctacgccgtcagctccaactaccac

SEQ ID NO: 125 - DNA, 29 bp, synthetic construct
  misc_feature 1..29: note = Description of Artificial Sequence: Syntheticprobe
  source 1..29: mol_type = other DNA
cttgcctacgccaccagctccaactacca

SEQ ID NO: 126 - DNA, 29 bp, synthetic construct
  misc_feature 1..29: note = Description of Artificial Sequence: Syntheticprobe
  source 1..29: mol_type = other DNA
cttgcctacgccacaagctccaactacca

SEQ ID NO: 127 - DNA, 30 bp, synthetic construct; identical to SEQ ID NO: 95
  misc_feature 1..30: note = Description of Artificial Sequence: Syntheticprobe
  source 1..30: mol_type = other DNA

SEQ ID NO: 128 - DNA, 30 bp, synthetic construct
  misc_feature 1..30: note = Description of Artificial Sequence: Syntheticprobe
  source 1..30: mol_type = other DNA
gcactcttgcctacgccacaagctccaact

SEQ ID NO: 129 - DNA, 30 bp, synthetic construct; identical to SEQ ID NO: 97
  misc_feature 1..30: note = Description of Artificial Sequence: Syntheticprobe
  source 1..30: mol_type = other DNA

SEQ ID NO: 130 - DNA, 30 bp, synthetic construct
  misc_feature 1..30: note = Description of Artificial Sequence: Syntheticprobe
  source 1..30: mol_type = other DNA
tcttgcctacgccacaagctccaactacca

SEQ ID NO: 131 - DNA, 30 bp, synthetic construct; identical to SEQ ID NO: 99
  misc_feature 1..30: note = Description of Artificial Sequence: Syntheticprobe
  source 1..30: mol_type = other DNA

SEQ ID NO: 132 - DNA, 30 bp, synthetic construct
  misc_feature 1..30: note = Description of Artificial Sequence: Syntheticprobe
  source 1..30: mol_type = other DNA
cttgcctacgccacaagctccaactaccac

SEQ ID NO: 133 - DNA, 50 bp, synthetic construct
  misc_feature 1..50: note = Description of Artificial Sequence: Syntheticprobe
  source 1..50: mol_type = other DNA
catgtcaagatcacagattttgggctggccaaactgctgggtgcggaaga

SEQ ID NO: 134 - DNA, 50 bp, synthetic construct
  misc_feature 1..50: note = Description of Artificial Sequence: Syntheticprobe
  source 1..50: mol_type = other DNA
catgtcaagatcacagattttgggagggccaaactgctgggtgcggaaga

SEQ ID NO: 135 - DNA, 51 bp, synthetic construct
  misc_feature 1..51: note = Description of Artificial Sequence: Syntheticprobe
  source 1..51: mol_type = other DNA
catgtcaagatcacagattttgggctggccaaactgctgggtgcggaagag

SEQ ID NO: 136 - DNA, 51 bp, synthetic construct
  misc_feature 1..51: note = Description of Artificial Sequence: Syntheticprobe
  source 1..51: mol_type = other DNA
catgtcaagatcacagattttgggagggccaaactgctgggtgcggaagag

SEQ ID NO: 137 - DNA, 52 bp, synthetic construct
  misc_feature 1..52: note = Description of Artificial Sequence: Syntheticprobe
  source 1..52: mol_type = other DNA
gcatgtcaagatcacagattttgggctggccaaactgctgggtgcggaagag

SEQ ID NO: 138 - DNA, 52 bp, synthetic construct
  misc_feature 1..52: note = Description of Artificial Sequence: Syntheticprobe
  source 1..52: mol_type = other DNA
gcatgtcaagatcacagattttgggagggccaaactgctgggtgcggaagag

SEQ ID NO: 139 - DNA, 53 bp, synthetic construct
  misc_feature 1..53: note = Description of Artificial Sequence: Syntheticprobe
  source 1..53: mol_type = other DNA
gcatgtcaagatcacagattttgggctggccaaactgctgggtgcggaagaga

SEQ ID NO: 140 - DNA, 53 bp, synthetic construct
  misc_feature 1..53: note = Description of Artificial Sequence: Syntheticprobe
  source 1..53: mol_type = other DNA
gcatgtcaagatcacagattttgggagggccaaactgctgggtgcggaagaga

SEQ ID NO: 141 - DNA, 57 bp, synthetic construct
  misc_feature 1..57: note = Description of Artificial Sequence: Syntheticprobe
  source 1..57: mol_type = other DNA
cagcatgtcaagatcacagattttgggctggccaaactgctgggtgcggaagagaaa

SEQ ID NO: 142 - DNA, 57 bp, synthetic construct
  misc_feature 1..57: note = Description of Artificial Sequence: Syntheticprobe
  source 1..57: mol_type = other DNA
cagcatgtcaagatcacagattttgggagggccaaactgctgggtgcggaagagaaa

SEQ ID NO: 143 - DNA, 51 bp, synthetic construct
  misc_feature 1..51: note = Description of Artificial Sequence: Syntheticprobe
  source 1..51: mol_type = other DNA
ctgcctcacctccaccgtgcagctcatcacgcagctcatgcccttcggctg

SEQ ID NO: 144 - DNA, 51 bp, synthetic construct
  misc_feature 1..51: note = Description of Artificial Sequence: Syntheticprobe
  source 1..51: mol_type = other DNA
ctgcctcacctccaccgtgcagctcatcatgcagctcatgcccttcggctg

SEQ ID NO: 145 - DNA, 51 bp, synthetic construct
  misc_feature 1..51: note = Description of Artificial Sequence: Syntheticprobe
  source 1..51: mol_type = other DNA
ctcacctccaccgtgcagctcatcacgcagctcatgcccttcggctgcctc

SEQ ID NO: 146 - DNA, 51 bp, synthetic construct
  misc_feature 1..51: note = Description of Artificial Sequence: Syntheticprobe
  source 1..51: mol_type = other DNA
ctcacctccaccgtgcagctcatcatgcagctcatgcccttcggctgcctc

SEQ ID NO: 147 - DNA, 52 bp, synthetic construct
  misc_feature 1..52: note = Description of Artificial Sequence: Syntheticprobe
  source 1..52: mol_type = other DNA
atctgcctcacctccaccgtgcagctcatcacgcagctcatgcccttcggct

SEQ ID NO: 148 - DNA, 52 bp, synthetic construct
  misc_feature 1..52: note = Description of Artificial Sequence: Syntheticprobe
  source 1..52: mol_type = other DNA
atctgcctcacctccaccgtgcagctcatcatgcagctcatgcccttcggct

SEQ ID NO: 149 - DNA, 53 bp, synthetic construct
  misc_feature 1..53: note = Description of Artificial Sequence: Syntheticprobe
  source 1..53: mol_type = other DNA
atctgcctcacctccaccgtgcagctcatcacgcagctcatgcccttcggctg

SEQ ID NO: 150 - DNA, 53 bp, synthetic construct
  misc_feature 1..53: note = Description of Artificial Sequence: Syntheticprobe
  source 1..53: mol_type = other DNA
atctgcctcacctccaccgtgcagctcatcatgcagctcatgcccttcggctg

SEQ ID NO: 151 - DNA, 54 bp, synthetic construct
  misc_feature 1..54: note = Description of Artificial Sequence: Syntheticprobe
  source 1..54: mol_type = other DNA
catctgcctcacctccaccgtgcagctcatcacgcagctcatgcccttcggctg

SEQ ID NO: 152 - DNA, 54 bp, synthetic construct
  misc_feature 1..54: note = Description of Artificial Sequence: Syntheticprobe
  source 1..54: mol_type = other DNA
catctgcctcacctccaccgtgcagctcatcatgcagctcatgcccttcggctg

SEQ ID NO: 153 - DNA, 50 bp, synthetic construct; identical to SEQ ID NO: 133
  misc_feature 1..50: note = Description of Artificial Sequence: Syntheticprobe
  source 1..50: mol_type = other DNA

SEQ ID NO: 154 - DNA, 50 bp, synthetic construct
  misc_feature 1..50: note = Description of Artificial Sequence: Syntheticprobe
  source 1..50: mol_type = other DNA
catgtcaagatcacagattttgggcgagccaaactgctgggtgcggaaga

SEQ ID NO: 155 - DNA, 51 bp, synthetic construct; identical to SEQ ID NO: 135
  misc_feature 1..51: note = Description of Artificial Sequence: Syntheticprobe
  source 1..51: mol_type = other DNA

SEQ ID NO: 156 - DNA, 51 bp, synthetic construct
  misc_feature 1..51: note = Description of Artificial Sequence: Syntheticprobe
  source 1..51: mol_type = other DNA
catgtcaagatcacagattttgggcgagccaaactgctgggtgcggaagag

SEQ ID NO: 157 - DNA, 52 bp, synthetic construct; identical to SEQ ID NO: 137
  misc_feature 1..52: note = Description of Artificial Sequence: Syntheticprobe
  source 1..52: mol_type = other DNA

SEQ ID NO: 158 - DNA, 52 bp, synthetic construct
  misc_feature 1..52: note = Description of Artificial Sequence: Syntheticprobe
  source 1..52: mol_type = other DNA
gcatgtcaagatcacagattttgggcgagccaaactgctgggtgcggaagag

SEQ ID NO: 159 - DNA, 53 bp, synthetic construct; identical to SEQ ID NO: 139
  misc_feature 1..53: note = Description of Artificial Sequence: Syntheticprobe
  source 1..53: mol_type = other DNA

SEQ ID NO: 160 - DNA, 53 bp, synthetic construct
  misc_feature 1..53: note = Description of Artificial Sequence: Syntheticprobe
  source 1..53: mol_type = other DNA
gcatgtcaagatcacagattttgggcgagccaaactgctgggtgcggaagaga

SEQ ID NO: 161 - DNA, 57 bp, synthetic construct; identical to SEQ ID NO: 141
  misc_feature 1..57: note = Description of Artificial Sequence: Syntheticprobe
  source 1..57: mol_type = other DNA

SEQ ID NO: 162 - DNA, 57 bp, synthetic construct
  misc_feature 1..57: note = Description of Artificial Sequence: Syntheticprobe
  source 1..57: mol_type = other DNA
cagcatgtcaagatcacagattttgggcgagccaaactgctgggtgcggaagagaaa

SEQ ID NO: 163 - DNA, 50 bp, synthetic construct; identical to SEQ ID NO: 133
  misc_feature 1..50: note = Description of Artificial Sequence: Syntheticprobe
  source 1..50: mol_type = other DNA

SEQ ID NO: 164 - DNA, 50 bp, synthetic construct
  misc_feature 1..50: note = Description of Artificial Sequence: Syntheticprobe
  source 1..50: mol_type = other DNA
catgtcaagatcacagattttgggcgtgccaaactgctgggtgcggaaga

SEQ ID NO: 165 - DNA, 51 bp, synthetic construct; identical to SEQ ID NO: 135
  misc_feature 1..51: note = Description of Artificial Sequence: Syntheticprobe
  source 1..51: mol_type = other DNA

SEQ ID NO: 166 - DNA, 51 bp, synthetic construct
  misc_feature 1..51: note = Description of Artificial Sequence: Syntheticprobe
  source 1..51: mol_type = other DNA
catgtcaagatcacagattttgggcgtgccaaactgctgggtgcggaagag

SEQ ID NO: 167 - DNA, 52 bp, synthetic construct; identical to SEQ ID NO: 137
  misc_feature 1..52: note = Description of Artificial Sequence: Syntheticprobe
  source 1..52: mol_type = other DNA

SEQ ID NO: 168 - DNA, 52 bp, synthetic construct
  misc_feature 1..52: note = Description of Artificial Sequence: Syntheticprobe
  source 1..52: mol_type = other DNA
gcatgtcaagatcacagattttgggcgtgccaaactgctgggtgcggaagag

SEQ ID NO: 169 - DNA, 50 bp, synthetic construct; identical to SEQ ID NO: 133
  misc_feature 1..50: note = Description of Artificial Sequence: Syntheticprobe
  source 1..50: mol_type = other DNA

SEQ ID NO: 170 - DNA, 50 bp, synthetic construct
  misc_feature 1..50: note = Description of Artificial Sequence: Syntheticprobe
  source 1..50: mol_type = other DNA
catgtcaagatcacagattttgggcgggccaaactgctgggtgcggaaga

SEQ ID NO: 171 - DNA, 51 bp, synthetic construct; identical to SEQ ID NO: 135
  misc_feature 1..51: note = Description of Artificial Sequence: Syntheticprobe
  source 1..51: mol_type = other DNA

SEQ ID NO: 172 - DNA, 51 bp, synthetic construct
  misc_feature 1..51: note = Description of Artificial Sequence: Syntheticprobe
  source 1..51: mol_type = other DNA
catgtcaagatcacagattttgggcgggccaaactgctgggtgcggaagag

SEQ ID NO: 173 - DNA, 50 bp, synthetic construct
  misc_feature 1..50: note = Description of Artificial Sequence: Syntheticprobe
  source 1..50: mol_type = other DNA
cgtcaaggcactcttgcctacgccaccagctccaactaccacaagtttat

SEQ ID NO: 174 - DNA, 50 bp, synthetic construct
  misc_feature 1..50: note = Description of Artificial Sequence: Syntheticprobe
  source 1..50: mol_type = other DNA
cgtcaaggcactcttgcctacgccatcagctccaactaccacaagtttat

SEQ ID NO: 175 - DNA, 51 bp, synthetic construct
  misc_feature 1..51: note = Description of Artificial Sequence: Syntheticprobe
  source 1..51: mol_type = other DNA
cgtcaaggcactcttgcctacgccaccagctccaactaccacaagtttata

SEQ ID NO: 176 - DNA, 51 bp, synthetic construct
  misc_feature 1..51: note = Description of Artificial Sequence: Syntheticprobe
  source 1..51: mol_type = other DNA
cgtcaaggcactcttgcctacgccatcagctccaactaccacaagtttata

SEQ ID NO: 177 - DNA, 52 bp, synthetic construct
  misc_feature 1..52: note = Description of Artificial Sequence: Syntheticprobe
  source 1..52: mol_type = other DNA
tcgtcaaggcactcttgcctacgccaccagctccaactaccacaagtttata

SEQ ID NO: 178 - DNA, 52 bp, synthetic construct
  misc_feature 1..52: note = Description of Artificial Sequence: Syntheticprobe
  source 1..52: mol_type = other DNA
tcgtcaaggcactcttgcctacgccatcagctccaactaccacaagtttata

SEQ ID NO: 179 - DNA, 53 bp, synthetic construct
  misc_feature 1..53: note = Description of Artificial Sequence: Syntheticprobe
  source 1..53: mol_type = other DNA
tcgtcaaggcactcttgcctacgccaccagctccaactaccacaagtttatat

SEQ ID NO: 180 - DNA, 53 bp, synthetic construct
  misc_feature 1..53: note = Description of Artificial Sequence: Syntheticprobe
  source 1..53: mol_type = other DNA
tcgtcaaggcactcttgcctacgccatcagctccaactaccacaagtttatat

SEQ ID NO: 181 - DNA, 54 bp, synthetic construct
  misc_feature 1..54: note = Description of Artificial Sequence: Syntheticprobe
  source 1..54: mol_type = other DNA
atcgtcaaggcactcttgcctacgccaccagctccaactaccacaagtttatat

SEQ ID NO: 182 - DNA, 54 bp, synthetic construct
  misc_feature 1..54: note = Description of Artificial Sequence: Syntheticprobe
  source 1..54: mol_type = other DNA
atcgtcaaggcactcttgcctacgccatcagctccaactaccacaagtttatat

SEQ ID NO: 183 - DNA, 55 bp, synthetic construct
  misc_feature 1..55: note = Description of Artificial Sequence: Syntheticprobe
  source 1..55: mol_type = other DNA
atcgtcaaggcactcttgcctacgccaccagctccaactaccacaagtttatatt

SEQ ID NO: 184 - DNA, 55 bp, synthetic construct
  misc_feature 1..55: note = Description of Artificial Sequence: Syntheticprobe
  source 1..55: mol_type = other DNA
atcgtcaaggcactcttgcctacgccatcagctccaactaccacaagtttatatt

SEQ ID NO: 185 - DNA, 56 bp, synthetic construct
  misc_feature 1..56: note = Description of Artificial Sequence: Syntheticprobe
  source 1..56: mol_type = other DNA
tatcgtcaaggcactcttgcctacgccaccagctccaactaccacaagtttatatt

SEQ ID NO: 186 - DNA, 56 bp, synthetic construct
  misc_feature 1..56: note = Description of Artificial Sequence: Syntheticprobe
  source 1..56: mol_type = other DNA
tatcgtcaaggcactcttgcctacgccatcagctccaactaccacaagtttatatt

SEQ ID NO: 187 - DNA, 57 bp, synthetic construct
  misc_feature 1..57: note = Description of Artificial Sequence: Syntheticprobe
  source 1..57: mol_type = other DNA
tatcgtcaaggcactcttgcctacgccaccagctccaactaccacaagtttatattc

SEQ ID NO: 188 - DNA, 57 bp, synthetic construct
  misc_feature 1..57: note = Description of Artificial Sequence: Syntheticprobe
  source 1..57: mol_type = other DNA
tatcgtcaaggcactcttgcctacgccatcagctccaactaccacaagtttatattc

SEQ ID NO: 189 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccaccagctccaactaccacaagtttatattc

SEQ ID NO: 190 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccatcagctccaactaccacaagtttatattc

SEQ ID NO: 191 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccaccagctccaactaccacaagtttatattca

SEQ ID NO: 192 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccatcagctccaactaccacaagtttatattca

SEQ ID NO: 193 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccaccagctccaactaccacaagtttatattca

SEQ ID NO: 194 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccatcagctccaactaccacaagtttatattca

SEQ ID NO: 195 - DNA, 50 bp, synthetic construct; identical to SEQ ID NO: 173
  misc_feature 1..50: note = Description of Artificial Sequence: Syntheticprobe
  source 1..50: mol_type = other DNA

SEQ ID NO: 196 - DNA, 50 bp, synthetic construct
  misc_feature 1..50: note = Description of Artificial Sequence: Syntheticprobe
  source 1..50: mol_type = other DNA
cgtcaaggcactcttgcctacgccagcagctccaactaccacaagtttat

SEQ ID NO: 197 - DNA, 51 bp, synthetic construct; identical to SEQ ID NO: 175
  misc_feature 1..51: note = Description of Artificial Sequence: Syntheticprobe
  source 1..51: mol_type = other DNA

SEQ ID NO: 198 - DNA, 51 bp, synthetic construct
  misc_feature 1..51: note = Description of Artificial Sequence: Syntheticprobe
  source 1..51: mol_type = other DNA
cgtcaaggcactcttgcctacgccagcagctccaactaccacaagtttata

SEQ ID NO: 199 - DNA, 52 bp, synthetic construct; identical to SEQ ID NO: 177
  misc_feature 1..52: note = Description of Artificial Sequence: Syntheticprobe
  source 1..52: mol_type = other DNA

SEQ ID NO: 200 - DNA, 52 bp, synthetic construct
  misc_feature 1..52: note = Description of Artificial Sequence: Syntheticprobe
  source 1..52: mol_type = other DNA
tcgtcaaggcactcttgcctacgccagcagctccaactaccacaagtttata

SEQ ID NO: 201 - DNA, 53 bp, synthetic construct; identical to SEQ ID NO: 179
  misc_feature 1..53: note = Description of Artificial Sequence: Syntheticprobe
  source 1..53: mol_type = other DNA

SEQ ID NO: 202 - DNA, 53 bp, synthetic construct
  misc_feature 1..53: note = Description of Artificial Sequence: Syntheticprobe
  source 1..53: mol_type = other DNA
tcgtcaaggcactcttgcctacgccagcagctccaactaccacaagtttatat

SEQ ID NO: 203 - DNA, 54 bp, synthetic construct; identical to SEQ ID NO: 181
  misc_feature 1..54: note = Description of Artificial Sequence: Syntheticprobe
  source 1..54: mol_type = other DNA

SEQ ID NO: 204 - DNA, 54 bp, synthetic construct
  misc_feature 1..54: note = Description of Artificial Sequence: Syntheticprobe
  source 1..54: mol_type = other DNA
atcgtcaaggcactcttgcctacgccagcagctccaactaccacaagtttatat

SEQ ID NO: 205 - DNA, 55 bp, synthetic construct; identical to SEQ ID NO: 183
  misc_feature 1..55: note = Description of Artificial Sequence: Syntheticprobe
  source 1..55: mol_type = other DNA

SEQ ID NO: 206 - DNA, 55 bp, synthetic construct
  misc_feature 1..55: note = Description of Artificial Sequence: Syntheticprobe
  source 1..55: mol_type = other DNA
atcgtcaaggcactcttgcctacgccagcagctccaactaccacaagtttatatt

SEQ ID NO: 207 - DNA, 56 bp, synthetic construct; identical to SEQ ID NO: 185
  misc_feature 1..56: note = Description of Artificial Sequence: Syntheticprobe
  source 1..56: mol_type = other DNA

SEQ ID NO: 208 - DNA, 56 bp, synthetic construct
  misc_feature 1..56: note = Description of Artificial Sequence: Syntheticprobe
  source 1..56: mol_type = other DNA
tatcgtcaaggcactcttgcctacgccagcagctccaactaccacaagtttatatt

SEQ ID NO: 209 - DNA, 57 bp, synthetic construct; identical to SEQ ID NO: 187
  misc_feature 1..57: note = Description of Artificial Sequence: Syntheticprobe
  source 1..57: mol_type = other DNA

SEQ ID NO: 210 - DNA, 57 bp, synthetic construct
  misc_feature 1..57: note = Description of Artificial Sequence: Syntheticprobe
  source 1..57: mol_type = other DNA
tatcgtcaaggcactcttgcctacgccagcagctccaactaccacaagtttatattc

SEQ ID NO: 211 - DNA, 58 bp, synthetic construct; identical to SEQ ID NO: 189
  misc_feature 1..58: note = Description of Artificial Sequence: Syntheticprobe
  source 1..58: mol_type = other DNA

SEQ ID NO: 212 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccagcagctccaactaccacaagtttatattc

SEQ ID NO: 213 - DNA, 59 bp, synthetic construct; identical to SEQ ID NO: 191
  misc_feature 1..59: note = Description of Artificial Sequence: Syntheticprobe
  source 1..59: mol_type = other DNA

SEQ ID NO: 214 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccagcagctccaactaccacaagtttatattca

SEQ ID NO: 215 - DNA, 60 bp, synthetic construct; identical to SEQ ID NO: 193
  misc_feature 1..60: note = Description of Artificial Sequence: Syntheticprobe
  source 1..60: mol_type = other DNA

SEQ ID NO: 216 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccagcagctccaactaccacaagtttatattca

SEQ ID NO: 217 - DNA, 50 bp, synthetic construct; identical to SEQ ID NO: 173
  misc_feature 1..50: note = Description of Artificial Sequence: Syntheticprobe
  source 1..50: mol_type = other DNA

SEQ ID NO: 218 - DNA, 50 bp, synthetic construct
  misc_feature 1..50: note = Description of Artificial Sequence: Syntheticprobe
  source 1..50: mol_type = other DNA
cgtcaaggcactcttgcctacgccgcaagctccaactaccacaagtttat

SEQ ID NO: 219 - DNA, 51 bp, synthetic construct; identical to SEQ ID NO: 175
  misc_feature 1..51: note = Description of Artificial Sequence: Syntheticprobe
  source 1..51: mol_type = other DNA

SEQ ID NO: 220 - DNA, 51 bp, synthetic construct
  misc_feature 1..51: note = Description of Artificial Sequence: Syntheticprobe
  source 1..51: mol_type = other DNA
cgtcaaggcactcttgcctacgccgcaagctccaactaccacaagtttata

SEQ ID NO: 221 - DNA, 52 bp, synthetic construct; identical to SEQ ID NO: 177
  misc_feature 1..52: note = Description of Artificial Sequence: Syntheticprobe
  source 1..52: mol_type = other DNA

SEQ ID NO: 222 - DNA, 52 bp, synthetic construct
  misc_feature 1..52: note = Description of Artificial Sequence: Syntheticprobe
  source 1..52: mol_type = other DNA
tcgtcaaggcactcttgcctacgccgcaagctccaactaccacaagtttata

SEQ ID NO: 223 - DNA, 53 bp, synthetic construct; identical to SEQ ID NO: 179
  misc_feature 1..53: note = Description of Artificial Sequence: Syntheticprobe
  source 1..53: mol_type = other DNA

SEQ ID NO: 224 - DNA, 53 bp, synthetic construct
  misc_feature 1..53: note = Description of Artificial Sequence: Syntheticprobe
  source 1..53: mol_type = other DNA
tcgtcaaggcactcttgcctacgccgcaagctccaactaccacaagtttatat

SEQ ID NO: 225 - DNA, 54 bp, synthetic construct; identical to SEQ ID NO: 181
  misc_feature 1..54: note = Description of Artificial Sequence: Syntheticprobe
  source 1..54: mol_type = other DNA

SEQ ID NO: 226 - DNA, 54 bp, synthetic construct
  misc_feature 1..54: note = Description of Artificial Sequence: Syntheticprobe
  source 1..54: mol_type = other DNA
atcgtcaaggcactcttgcctacgccgcaagctccaactaccacaagtttatat

SEQ ID NO: 227 - DNA, 55 bp, synthetic construct; identical to SEQ ID NO: 183
  misc_feature 1..55: note = Description of Artificial Sequence: Syntheticprobe
  source 1..55: mol_type = other DNA

SEQ ID NO: 228 - DNA, 55 bp, synthetic construct
  misc_feature 1..55: note = Description of Artificial Sequence: Syntheticprobe
  source 1..55: mol_type = other DNA
atcgtcaaggcactcttgcctacgccgcaagctccaactaccacaagtttatatt

SEQ ID NO: 229 - DNA, 56 bp, synthetic construct; identical to SEQ ID NO: 185
  misc_feature 1..56: note = Description of Artificial Sequence: Syntheticprobe
  source 1..56: mol_type = other DNA

SEQ ID NO: 230 - DNA, 56 bp, synthetic construct
  misc_feature 1..56: note = Description of Artificial Sequence: Syntheticprobe
  source 1..56: mol_type = other DNA
tatcgtcaaggcactcttgcctacgccgcaagctccaactaccacaagtttatatt

SEQ ID NO: 231 - DNA, 57 bp, synthetic construct; identical to SEQ ID NO: 187
  misc_feature 1..57: note = Description of Artificial Sequence: Syntheticprobe
  source 1..57: mol_type = other DNA

SEQ ID NO: 232 - DNA, 57 bp, synthetic construct
  misc_feature 1..57: note = Description of Artificial Sequence: Syntheticprobe
  source 1..57: mol_type = other DNA
tatcgtcaaggcactcttgcctacgccgcaagctccaactaccacaagtttatattc

SEQ ID NO: 233 - DNA, 58 bp, synthetic construct; identical to SEQ ID NO: 189
  misc_feature 1..58: note = Description of Artificial Sequence: Syntheticprobe
  source 1..58: mol_type = other DNA

SEQ ID NO: 234 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccgcaagctccaactaccacaagtttatattc

SEQ ID NO: 235 - DNA, 59 bp, synthetic construct; identical to SEQ ID NO: 191
  misc_feature 1..59: note = Description of Artificial Sequence: Syntheticprobe
  source 1..59: mol_type = other DNA

SEQ ID NO: 236 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccgcaagctccaactaccacaagtttatattca

SEQ ID NO: 237 - DNA, 60 bp, synthetic construct; identical to SEQ ID NO: 193
  misc_feature 1..60: note = Description of Artificial Sequence: Syntheticprobe
  source 1..60: mol_type = other DNA

SEQ ID NO: 238 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccgcaagctccaactaccacaagtttatattca

SEQ ID NO: 239 - DNA, 50 bp, synthetic construct; identical to SEQ ID NO: 173
  misc_feature 1..50: note = Description of Artificial Sequence: Syntheticprobe
  source 1..50: mol_type = other DNA

SEQ ID NO: 240 - DNA, 50 bp, synthetic construct
  misc_feature 1..50: note = Description of Artificial Sequence: Syntheticprobe
  source 1..50: mol_type = other DNA
cgtcaaggcactcttgcctacgccgtcagctccaactaccacaagtttat

SEQ ID NO: 241 - DNA, 51 bp, synthetic construct; identical to SEQ ID NO: 175
  misc_feature 1..51: note = Description of Artificial Sequence: Syntheticprobe
  source 1..51: mol_type = other DNA

SEQ ID NO: 242 - DNA, 51 bp, synthetic construct
  misc_feature 1..51: note = Description of Artificial Sequence: Syntheticprobe
  source 1..51: mol_type = other DNA
cgtcaaggcactcttgcctacgccgtcagctccaactaccacaagtttata

SEQ ID NO: 243 - DNA, 52 bp, synthetic construct; identical to SEQ ID NO: 177
  misc_feature 1..52: note = Description of Artificial Sequence: Syntheticprobe
  source 1..52: mol_type = other DNA

SEQ ID NO: 244 - DNA, 52 bp, synthetic construct
  misc_feature 1..52: note = Description of Artificial Sequence: Syntheticprobe
  source 1..52: mol_type = other DNA
tcgtcaaggcactcttgcctacgccgtcagctccaactaccacaagtttata

SEQ ID NO: 245 - DNA, 53 bp, synthetic construct; identical to SEQ ID NO: 179
  misc_feature 1..53: note = Description of Artificial Sequence: Syntheticprobe
  source 1..53: mol_type = other DNA

SEQ ID NO: 246 - DNA, 53 bp, synthetic construct
  misc_feature 1..53: note = Description of Artificial Sequence: Syntheticprobe
  source 1..53: mol_type = other DNA
tcgtcaaggcactcttgcctacgccgtcagctccaactaccacaagtttatat

SEQ ID NO: 247 - DNA, 54 bp, synthetic construct; identical to SEQ ID NO: 181
  misc_feature 1..54: note = Description of Artificial Sequence: Syntheticprobe
  source 1..54: mol_type = other DNA

SEQ ID NO: 248 - DNA, 54 bp, synthetic construct
  misc_feature 1..54: note = Description of Artificial Sequence: Syntheticprobe
  source 1..54: mol_type = other DNA
atcgtcaaggcactcttgcctacgccgtcagctccaactaccacaagtttatat

SEQ ID NO: 249 - DNA, 55 bp, synthetic construct; identical to SEQ ID NO: 183
  misc_feature 1..55: note = Description of Artificial Sequence: Syntheticprobe
  source 1..55: mol_type = other DNA

SEQ ID NO: 250 - DNA, 55 bp, synthetic construct
  misc_feature 1..55: note = Description of Artificial Sequence: Syntheticprobe
  source 1..55: mol_type = other DNA
atcgtcaaggcactcttgcctacgccgtcagctccaactaccacaagtttatatt

SEQ ID NO: 251 - DNA, 56 bp, synthetic construct; identical to SEQ ID NO: 185
  misc_feature 1..56: note = Description of Artificial Sequence: Syntheticprobe
  source 1..56: mol_type = other DNA

SEQ ID NO: 252 - DNA, 56 bp, synthetic construct
  misc_feature 1..56: note = Description of Artificial Sequence: Syntheticprobe
  source 1..56: mol_type = other DNA
tatcgtcaaggcactcttgcctacgccgtcagctccaactaccacaagtttatatt

SEQ ID NO: 253 - DNA, 57 bp, synthetic construct; identical to SEQ ID NO: 187
  misc_feature 1..57: note = Description of Artificial Sequence: Syntheticprobe
  source 1..57: mol_type = other DNA

SEQ ID NO: 254 - DNA, 57 bp, synthetic construct
  misc_feature 1..57: note = Description of Artificial Sequence: Syntheticprobe
  source 1..57: mol_type = other DNA
tatcgtcaaggcactcttgcctacgccgtcagctccaactaccacaagtttatattc

SEQ ID NO: 255 - DNA, 58 bp, synthetic construct; identical to SEQ ID NO: 189
  misc_feature 1..58: note = Description of Artificial Sequence: Syntheticprobe
  source 1..58: mol_type = other DNA

SEQ ID NO: 256 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccgtcagctccaactaccacaagtttatattc

SEQ ID NO: 257 - DNA, 59 bp, synthetic construct; identical to SEQ ID NO: 191
  misc_feature 1..59: note = Description of Artificial Sequence: Syntheticprobe
  source 1..59: mol_type = other DNA

SEQ ID NO: 258 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccgtcagctccaactaccacaagtttatattca

SEQ ID NO: 259 - DNA, 60 bp, synthetic construct; identical to SEQ ID NO: 193
  misc_feature 1..60: note = Description of Artificial Sequence: Syntheticprobe
  source 1..60: mol_type = other DNA

SEQ ID NO: 260 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccgtcagctccaactaccacaagtttatattca

SEQ ID NO: 261 - DNA, 50 bp, synthetic construct; identical to SEQ ID NO: 173
  misc_feature 1..50: note = Description of Artificial Sequence: Syntheticprobe
  source 1..50: mol_type = other DNA

SEQ ID NO: 262 - DNA, 50 bp, synthetic construct
  misc_feature 1..50: note = Description of Artificial Sequence: Syntheticprobe
  source 1..50: mol_type = other DNA
cgtcaaggcactcttgcctacgccacaagctccaactaccacaagtttat

SEQ ID NO: 263 - DNA, 51 bp, synthetic construct; identical to SEQ ID NO: 175
  misc_feature 1..51: note = Description of Artificial Sequence: Syntheticprobe
  source 1..51: mol_type = other DNA

SEQ ID NO: 264 - DNA, 51 bp, synthetic construct
  misc_feature 1..51: note = Description of Artificial Sequence: Syntheticprobe
  source 1..51: mol_type = other DNA
cgtcaaggcactcttgcctacgccacaagctccaactaccacaagtttata

SEQ ID NO: 265 - DNA, 52 bp, synthetic construct; identical to SEQ ID NO: 177
  misc_feature 1..52: note = Description of Artificial Sequence: Syntheticprobe
  source 1..52: mol_type = other DNA

SEQ ID NO: 266 - DNA, 52 bp, synthetic construct
  misc_feature 1..52: note = Description of Artificial Sequence: Syntheticprobe
  source 1..52: mol_type = other DNA
tcgtcaaggcactcttgcctacgccacaagctccaactaccacaagtttata

SEQ ID NO: 267 - DNA, 53 bp, synthetic construct; identical to SEQ ID NO: 179
  misc_feature 1..53: note = Description of Artificial Sequence: Syntheticprobe
  source 1..53: mol_type = other DNA

SEQ ID NO: 268 - DNA, 53 bp, synthetic construct
  misc_feature 1..53: note = Description of Artificial Sequence: Syntheticprobe
  source 1..53: mol_type = other DNA
tcgtcaaggcactcttgcctacgccacaagctccaactaccacaagtttatat

SEQ ID NO: 269 - DNA, 54 bp, synthetic construct; identical to SEQ ID NO: 181
  misc_feature 1..54: note = Description of Artificial Sequence: Syntheticprobe
  source 1..54: mol_type = other DNA

SEQ ID NO: 270 - DNA, 54 bp, synthetic construct
  misc_feature 1..54: note = Description of Artificial Sequence: Syntheticprobe
  source 1..54: mol_type = other DNA
atcgtcaaggcactcttgcctacgccacaagctccaactaccacaagtttatat

SEQ ID NO: 271 - DNA, 55 bp, synthetic construct; identical to SEQ ID NO: 183
  misc_feature 1..55: note = Description of Artificial Sequence: Syntheticprobe
  source 1..55: mol_type = other DNA

SEQ ID NO: 272 - DNA, 55 bp, synthetic construct
  misc_feature 1..55: note = Description of Artificial Sequence: Syntheticprobe
  source 1..55: mol_type = other DNA
atcgtcaaggcactcttgcctacgccacaagctccaactaccacaagtttatatt

SEQ ID NO: 273 - DNA, 56 bp, synthetic construct; identical to SEQ ID NO: 185
  misc_feature 1..56: note = Description of Artificial Sequence: Syntheticprobe
  source 1..56: mol_type = other DNA

SEQ ID NO: 274 - DNA, 56 bp, synthetic construct
  misc_feature 1..56: note = Description of Artificial Sequence: Syntheticprobe
  source 1..56: mol_type = other DNA
tatcgtcaaggcactcttgcctacgccacaagctccaactaccacaagtttatatt

SEQ ID NO: 275 - DNA, 57 bp, synthetic construct; identical to SEQ ID NO: 187
  misc_feature 1..57: note = Description of Artificial Sequence: Syntheticprobe
  source 1..57: mol_type = other DNA

SEQ ID NO: 276 - DNA, 57 bp, synthetic construct
  misc_feature 1..57: note = Description of Artificial Sequence: Syntheticprobe
  source 1..57: mol_type = other DNA
tatcgtcaaggcactcttgcctacgccacaagctccaactaccacaagtttatattc

SEQ ID NO: 277 - DNA, 58 bp, synthetic construct; identical to SEQ ID NO: 189
  misc_feature 1..58: note = Description of Artificial Sequence: Syntheticprobe
  source 1..58: mol_type = other DNA

SEQ ID NO: 278 - DNA, 58 bp, synthetic construct
  misc_feature 1..58: note = Description of Artificial Sequence: Syntheticprobe
  source 1..58: mol_type = other DNA
gtatcgtcaaggcactcttgcctacgccacaagctccaactaccacaagtttatattc

SEQ ID NO: 279 - DNA, 59 bp, synthetic construct; identical to SEQ ID NO: 191
  misc_feature 1..59: note = Description of Artificial Sequence: Syntheticprobe
  source 1..59: mol_type = other DNA

SEQ ID NO: 280 - DNA, 59 bp, synthetic construct
  misc_feature 1..59: note = Description of Artificial Sequence: Syntheticprobe
  source 1..59: mol_type = other DNA
gtatcgtcaaggcactcttgcctacgccacaagctccaactaccacaagtttatattca

SEQ ID NO: 281 - DNA, 60 bp, synthetic construct; identical to SEQ ID NO: 193
  misc_feature 1..60: note = Description of Artificial Sequence: Syntheticprobe
  source 1..60: mol_type = other DNA

SEQ ID NO: 282 - DNA, 60 bp, synthetic construct
  misc_feature 1..60: note = Description of Artificial Sequence: Syntheticprobe
  source 1..60: mol_type = other DNA
tgtatcgtcaaggcactcttgcctacgccacaagctccaactaccacaagtttatattca

SEQ ID NO: 283 - DNA, 30 bp, synthetic construct
  misc_feature 1..30: note = Description of Artificial Sequence: Syntheticprobe
  source 1..30: mol_type = other DNA
taaaaataggtgattttggtctagctacag

SEQ ID NO: 284 - DNA, 24 bp, synthetic construct
  misc_feature 1..24: note = Description of Artificial Sequence: Syntheticprobe
  source 1..24: mol_type = other DNA
atgtcaagatcacagattttgggc

SEQ ID NO: 285 - DNA, 20 bp, synthetic construct
  misc_feature 1..20: note = Description of Artificial Sequence: Syntheticprobe
  source 1..20: mol_type = other DNA
ctccaccgtgcagctcatca

SEQ ID NO: 286 - DNA, 26 bp, synthetic construct
  misc_feature 1..26: note = Description of Artificial Sequence: Syntheticprimer
  source 1..26: mol_type = other DNA
ataggtgattttggtctagctacagt

SEQ ID NO: 287 - DNA, 26 bp, synthetic construct
  misc_feature 1..26: note = Description of Artificial Sequence: Syntheticprimer
  source 1..26: mol_type = other DNA
ataggtgattttggtctagctacaga

SEQ ID NO: 288 - DNA, 21 bp, synthetic construct
  misc_feature 1..21: note = Description of Artificial Sequence: Syntheticprimer
  source 1..21: mol_type = other DNA
gacccactccatcgagatttc

SEQ ID NO: 289 - DNA, 24 bp, synthetic construct
  misc_feature 1..24: note = Description of Artificial Sequence: Syntheticprimer
  source 1..24: mol_type = other DNA
tgtcaagatcacagattttgggct

SEQ ID NO: 290 - DNA, 24 bp, synthetic construct
  misc_feature 1..24: note = Description of Artificial Sequence: Syntheticprimer
  source 1..24: mol_type = other DNA
tgtcaagatcacagattttgggcg

SEQ ID NO: 291 - DNA, 16 bp, synthetic construct
  misc_feature 1..16: note = Description of Artificial Sequence: Syntheticprimer
  source 1..16: mol_type = other DNA
acccagcagtttggcc

SEQ ID NO: 292 - DNA, 18 bp, synthetic construct
  misc_feature 1..18: note = Description of Artificial Sequence: Syntheticprimer
  source 1..18: mol_type = other DNA
caccgtgcagctcatcac

SEQ ID NO: 293 - DNA, 18 bp, synthetic construct
  misc_feature 1..18: note = Description of Artificial Sequence: Syntheticprimer
  source 1..18: mol_type = other DNA
caccgtgcagctcatcat

SEQ ID NO: 294 - DNA, 17 bp, synthetic construct
  misc_feature 1..17: note = Description of Artificial Sequence: Syntheticprimer
  source 1..17: mol_type = other DNA
cgaagggcatgagctgc

(19) United States
(12) Patent Application Publication
(10) Pub. No.: US 2023/0416806 A1
(43) Pub. Date: Dec. 28, 2023

(54) POLYMORPHISM DETECTION WITH INCREASED ACCURACY

(71) Applicant: Apton Biosystems, Inc., Pleasanton, CA (US)

(72) Inventors: Bryan P. STAKER, San Ramon, CA (US); Niandong LIU, San Ramon, CA (US); Manohar R. FURTADO, San Ramon, CA (US); Rixun FANG, Menlo Park, CA (US)

(21) Appl. No.: 17/955,426

(22) Filed: Sep. 28, 2022

Related U.S. Application Data
(63) Continuation of application No. 16/496,923, filed on Sep. 23, 2019, now abandoned.
(60) Provisional application No. 62/475,791, filed on Mar. 23, 2017.

(51) Int. Cl. C12Q 1/6827; C12Q 1/6837

(52) U.S. Cl. CPC C12Q 1/6827; C12Q 1/6837; C12Q 2533/107; C12Q 2537/155; C12Q 2565/514; C12Q 2565/518

(57) ABSTRACT

The invention relates to methods and compositions for the detection and quantification of nucleotide sequence variants, such as genetic polymorphisms, with decreased error and increased sensitivity, including single molecule detection. Detection of genetic polymorphisms, including single nucleotide polymorphisms (SNPs), is highly useful for the study of physiology, disease, phylogeny and forensics. Current methods for the detection and identification of nucleic acid sequence variants, such as genetic polymorphisms, lack the sensitivity to accurately detect low incidence mutations, sequence variants or alleles. Detection techniques for highly multiplexed single molecule identification and quantification of analytes using optical systems are disclosed. Analytes include, but are not limited to, nucleic acid, such as DNA and RNA molecules, with and without modifications. Techniques described herein include use of specific and non-specific probes complementary to nucleic acids of interest for detailed characterization of nucleotide sequence variants and highly multiplexed single molecule identification and quantification.

### CROSS-REFERENCE TO RELATED APPLICATIONS

This application is a continuation of U.S. application Ser. No. 16/496,923, filed Sep. 23, 2019, which is a 371 National Stage application of PCT/US2018/23310, filed Mar. 20, 2018, which claims the benefit of U.S. Provisional Application No. 62/475,791, filed Mar. 23, 2017, all of which are hereby incorporated in their entireties by reference.

### SEQUENCE LISTING

The instant application contains a Sequence Listing which has been submitted electronically in ASCII format and is hereby incorporated by reference in its entirety. Said ASCII copy, created on Sep. 28, 2022, is named 55495-805-301 SL.xml and is 388,232 bytes.

### BACKGROUND

### Field of the Invention

The invention relates to methods and compositions for the detection and quantification of nucleic acid sequences and nucleotide sequence variants, including genetic polymorphisms, with decreased error and increased sensitivity, including single molecule detection. Detection of genetic polymorphisms, including single nucleotide polymorphisms (SNPs) and Indels (insertion-deletions) is highly useful for the study of physiology, disease, phylogeny and forensics. Single-nucleotide polymorphisms and Indels are the most common forms of sequence variation between individuals. Analysis of this variation offers an opportunity to understand the genetic basis of disease, response to therapeutics and disease progression and is a driving force behind modern pharmacogenomics and disease management practices. Accurate, high throughput, and cost effective methods to analyze genetic variation are crucial to fully utilize the medical value of the DNA sequence data that has been generated in the human genome project.

### Description of the Related Art

Current methods for the detection and identification of nucleic acid sequence variants, such as genetic polymorphisms, lack the sensitivity to accurately detect low incidence mutations sequence variants or alleles. Furthermore, current methods are limited in their capacity for identification and quantification of sequence variants of a large number of loci. Current methods often generate errors during analyte detection and quantification due to conditions such as weak signal detection, false positives, and other mistakes. These errors may result in the misidentification and inaccurate quantification of nucleic acid analytes, particularly for rare sequence variants. Therefore, novel more sensitive and efficient approaches for the detection of rare or low incidence mutations are needed.

### SUMMARY OF THE INVENTION

Disclosed herein are methods of detecting at least one target nucleotide sequence variant suspected of being present in a sample. In certain embodiments, the application describes methods of detecting at least one target nucleotide sequence variant suspected of being present in a sample, comprising: distributing a plurality of oligonucleotides on a substrate such that individual oligonucleotides bind to the substrate at spatially separate regions; carrying out on the substrate a target nucleotide sequence variant identification assay, wherein the sequence variant identification assay comprises performing at least M detection cycles to generate a signal detection sequence, wherein M is at least two, each cycle comprising: contacting the plurality of oligonucleotides with a probe comprising a detection label, wherein the probe binds preferentially to one of the at least one target nucleotide sequence variants or a barcode sequence bound to one of the at least one target nucleotide sequence variants; washing the surface of the substrate to remove unbound barcode probes; detecting the identity and location of the detection label on the substrate, and if the cycle number is less than M, removing the barcode probe from the barcode moiety; and analyzing the signal detection sequence generated by the M cycles at the spatially separate locations on the substrate to determine the presence or absence of the at least one target nucleotide sequence variant of interest.

In certain embodiments, the application describes methods of identifying at least one target nucleotide sequence variant suspected of being present in a sample, comprising: distributing a plurality of oligonucleotides comprising N distinct nucleotide sequence variants on a substrate such that each distinct nucleotide sequence variant of the N distinct nucleotide sequence variants is immobilized on a solid substrate in a location that is spatially separate from any other distinct target analyte of the N distinct target analytes carrying out on the substrate a target nucleotide sequence variant identification assay for identifying at least one of N distinct nucleotide sequence variants, wherein the assay comprises: obtaining a plurality of ordered probe reagent sets, each of the ordered probe reagent sets comprising one or more probes directed to a defined subset of the N distinct nucleotide sequence variants, wherein each of the probes comprises a sequence complementary to an oligonucleotide comprising one of the nucleotide sequence variants, and wherein each of the probes is detectably labeled such that one probe is configured to detect one distinct nucleotide sequence variants; performing at least M cycles of probe binding and signal detection, each cycle comprising one or more passes, wherein a pass comprises use of at least one of the ordered probe reagent sets; detecting from the at least M cycles a presence or an absence of a plurality of signals from the spatially separate locations of the substrate; determining from the plurality of signals at least K bits of information per cycle for one or more of the N distinct nucleotide sequence variants, wherein the at least K bits of information are used to determine L total bits of information, wherein K×M=L bits of information and L>log₂ (N), and wherein the L bits of information are used to determine a presence or an absence of one or more of the N distinct nucleotide sequence variants.

In certain embodiments, the application discloses methods of detecting at least one target nucleotide sequence variant suspected of being present in a sample comprising providing a ligation reaction product of a target-dependent oligonucleotide ligation reaction performed on the sample, wherein the ligation reaction product comprises a plurality of oligonucleotides each comprising a substrate binding moiety and a barcode moiety; distributing the ligation reaction product on a substrate such that individual oligonucleotides bind to the substrate via the substrate binding moiety at spatially separate regions of the substrate; carrying out on the substrate a target nucleotide sequence variant identification assay, wherein the sequence variant identification assay comprises performing at least M detection cycles to generate a signal detection sequence, wherein M is at least two, each cycle comprising contacting the ligation reaction product with a barcode probe comprising a detection label, wherein the barcode probe binds to the barcode moiety when it is present on the substrate; washing the surface of the substrate to remove unbound barcode probes; detecting the identity and location of the detection label on the substrate; and if the cycle number is less than M, removing the barcode probe from the barcode moiety; and analyzing the signal detection sequence generated by the M cycles at the spatially separate locations on the substrate to determine the presence or absence of the at least one target nucleotide sequence variant of interest. In certain aspects, the ligation reaction product comprises an oligonucleotide comprising a sequence variant-specific oligonucleotide sequence, a locus-specific oligonucleotide sequence, a binding moiety, and a barcode moiety. In certain aspects, providing the ligation reaction product comprises carrying out the target-dependent oligonucleotide ligation reaction on the sample suspected of comprising at least one target nucleotide sequence variant. In certain aspects, the sample is an enriched nucleic acid sample suspected of comprising at least one target nucleotide sequence variant of a plurality of sequence variants at one of a plurality of target loci. In an aspect, the enriched nucleic acid sample is enriched by performing a reverse transcription reaction on a sample comprising RNA. In certain aspects, carrying out the target-dependent oligonucleotide ligation reaction comprises: providing a plurality of oligonucleotide probe sets, each set comprising a first oligonucleotide probe capable of hybridizing to one of a plurality of sequence variants at one of the plurality of target loci, wherein the probe is bound to a barcode moiety; a second oligonucleotide probe capable of hybridizing to a sequence adjacent to the sequence variant for a plurality of the plurality of sequence variants at the target locus, wherein the second oligonucleotide probe is bound to a substrate binding moiety; wherein the oligonucleotide probes in a particular set are suitable for ligation together when hybridized adjacent to one another on a corresponding target locus; contacting the sample with the N oligonucleotide probe sets to perform a hybridization reaction, wherein the first and second oligonucleotide probes hybridize at adjacent positions in a base-specific manner to their respective target sequences, if present in the sample; and contacting the hybridized sample with a ligase to perform a ligation reaction, wherein the hybridized first and second oligonucleotide probes from a ligation reaction product comprising the barcode moiety and the substrate binding moiety. In certain aspects, carrying out the target-dependent oligonucleotide ligation reaction comprises: hybridizing a sequence variant-specific oligonucleotide to a first region of a locus suspected of comprising the nucleotide sequence variant at the locus, wherein the sequence variant-specific oligonucleotide is bound to a barcode moiety, the barcode moiety comprising an identifier barcode sequence corresponding to a sequence variant at the locus, hybridizing a locus-specific oligonucleotide to a second region of the locus comprising a constant sequence at the locus, wherein the second oligonucleotide is bound to a substrate binding moiety, and wherein the first and second oligonucleotides are aligned for ligation when hybridized to the at least one target nucleotide sequence variant; and generating a ligation reaction product between the hybridized first oligonucleotide and the hybridized second oligonucleotide at the locus such that the ligation reaction product comprises a ligated oligonucleotide comprising both the barcode moiety and the substrate binding moiety. In certain aspects, the method further comprises the step of performing a denaturation reaction after generating the ligation reaction product to separate the ligation reaction product from the oligonucleotide comprising the target nucleotide sequence variant of interest prior to binding the ligation reaction product to the substrate. In an aspect, the barcode probe comprises a unique label between at least two different cycles. In certain aspects, analyzing the signal detection sequence comprises comparing the signal detection sequence with the anticipated signal detection sequence for the target nucleotide sequence variant of interest, and determining a probability score for the presence or absence of the target nucleotide sequence variant of interest based on the signal detection sequence. In an aspect, the analysis reduces an error due to misidentification of the target at least one of the M cycles. In an aspect, the misidentification event is due to a false positive or a false negative signal. In an aspect, the at least one target nucleotide sequence variant is an allele. In an aspect, the at least one sequence variant comprises a mutation. In an aspect the mutation is a low incidence genomic mutation of interest. In an aspect, the mutation is a deletion, an insertion, a replacement, or a rearrangement. In an aspect, the mutation is a single nucleotide polymorphism (SNP). In certain aspects of the methods, the false-positive rate for the detection of the at least one target nucleotide sequence variant of interest is less than 1 in 10⁶ wherein the target nucleotide sequence variant identification assay is performed simultaneously for a plurality of target nucleotide sequence variants at a plurality of loci, the assay comprising a plurality of the barcode probes that are unique for each of the plurality of target nucleotide sequence variants. In an aspect, the detection label is a fluorophore. In certain aspect of the methods, M is greater than 1, 2, 3, 4, 5, 10, 15, 20, 25, 30, 35, 40, 45, or In an aspect, M is sufficient to detect a barcode moiety bound to the substrate with a false positive detection rate of less than 1 in 10⁶. In certain aspects, the target-dependent oligonucleotide ligation reaction generates a plurality of distinct ligation products, the ligation products comprising a plurality of nucleotide sequence variants of interest at a plurality of distinct loci, each of the distinct ligation products each comprising a barcode probe comprising a unique identifier barcode sequence, wherein the nucleotide sequence variant identification assay is performed with a plurality of distinct barcode probes that each bind to a corresponding barcode sequence; and wherein the nucleotide sequence variant identification assay is performed for M number of cycles to produce an false positive rate of less than 1 in 10⁶ for the detection of each sequence variant of interest at the plurality of distinct loci. In certain embodiments, the application describes methods of identifying at least one target nucleotide sequence variant suspected of being present in a sample, comprising providing a ligation reaction product of a target-dependent oligonucleotide ligation reaction performed on the sample, wherein the ligation reaction product comprises a plurality of oligonucleotides each comprising a substrate binding moiety and a barcode moiety; distributing the ligation reaction product on a substrate such that individual oligonucleotides bind to the substrate via the substrate binding moiety at spatially separate regions of the substrate; carrying out on the substrate a target nucleotide sequence variant identification assay for identifying at least one of N nucleotide sequence variants, wherein the assay comprises: providing at least M sets of barcode probes for performing at least M cycles of the assay, each set comprising N unique barcode binding moieties capable of binding preferentially to a corresponding one of the N barcode moieties, each barcode probe set comprising a detection label for generating K bits of information per cycle; performing at least M detection cycles to generate a signal detection sequence at a plurality of locations on the substrate, wherein M is at least two, each cycle comprising contacting the substrate bound to the ligation reaction products with the barcode probe set corresponding with the cycle number; washing the surface of the substrate to remove unbound barcode probes; detecting the presence or absence of a plurality of signals from the spatially separate regions of the substrate; and if the cycle number is less than M, performing a denaturation reaction to remove the barcode probe from the barcode moiety; and determining from the at least M detection cycles L total bits of information, wherein K×M=L and L>log₂ (N), and wherein the L bits of information are used to identify one or more of the N nucleotide sequence variants. In certain aspects, the ligation reaction product comprises an oligonucleotide comprising a sequence variant-specific oligonucleotide sequence, a locus-specific oligonucleotide sequence, a binding moiety, and a barcode moiety. In an aspect, providing the ligation reaction product comprises carrying out the target-dependent oligonucleotide ligation reaction on the sample suspected of comprising at least one target nucleotide sequence variant. In certain aspects, the sample is an enriched nucleic acid sample suspected of comprising at least one target nucleotide sequence variant of a plurality of sequence variants at one of a plurality of target loci. In certain aspects, carrying out the target-dependent oligonucleotide ligation reaction comprises: providing N oligonucleotide probe sets, each set comprising a first oligonucleotide probe capable of hybridizing to one of a plurality of sequence variants at one of the plurality of target loci, wherein the probe is bound to a barcode moiety; a second oligonucleotide probe capable of hybridizing to a sequence adjacent to the sequence variant for a plurality of the plurality of sequence variants at the target locus, wherein the second oligonucleotide probe is bound to a substrate binding moiety; wherein the oligonucleotide probes in a particular set are suitable for ligation together when hybridized adjacent to one another on a corresponding target locus; contacting the sample with the N oligonucleotide probe sets to perform a hybridization reaction, wherein the first and second oligonucleotide probes hybridize at adjacent positions in a base-specific manner to their respective target sequences, if present in the sample; and contacting the hybridized sample with a ligase to perform a ligation reaction, wherein the hybridized first and second oligonucleotide probes from a ligation reaction product comprising the barcode moiety and the substrate binding moiety. In certain aspects, carrying out the target-dependent oligonucleotide ligation reaction comprises: hybridizing a sequence variant-specific oligonucleotide to a first region of a locus suspected of comprising the nucleotide sequence variant at the locus, wherein the sequence variant-specific oligonucleotide is bound to a barcode moiety, the barcode moiety comprising an identifier barcode sequence corresponding to a sequence variant at the locus, hybridizing a locus-specific oligonucleotide to a second region of the locus comprising a constant sequence at the locus, wherein the second oligonucleotide is bound to a substrate binding moiety, and wherein the first and second oligonucleotides are aligned for ligation when hybridized to the at least one target nucleotide sequence variant; and generating a ligation reaction product between the hybridized first oligonucleotide and the hybridized second oligonucleotide at the locus such that the ligation reaction product comprises a ligated oligonucleotide comprising both the barcode moiety and the substrate binding moiety. In an aspect, the nucleotide variant identification assay comprises determining L total bits of information such that L is sufficient to reduce a false positive error rate of detection to less than 1 in 10⁶. In an aspect, L is a function of the misidentification rate for a target at each cycle. In an aspect, misidentification rate comprises the non-binding rate and the false binding rate of the probe set to the barcode. In an aspect, the assay determines the presence or absence of the one or more N nucleotide sequence variants. In an aspect, the assay determines a quantity of the one or more N nucleotide sequence variants. In an aspect, the at least one of the M barcode binding moieties comprises a plurality of detection labels across the M sets of barcode probes. In an aspect, the nucleotide sequence variant is an allele at the locus. In an aspect, the locus comprises at least two alleles, and wherein identifying one or more of the N nucleotide sequence variants comprises identifying the presence or absence of one of the at least two alleles at the locus in the sample. In an aspect, the target nucleotide sequence variant comprises a single nucleotide polymorphism. In an aspect, the nucleotide sequence variant comprises a mutation. In an aspect, the mutation is a deletion, a replacement, or an insertion. In an aspect the mutation is a single nucleotide polymorphism. In an aspect, L comprises bits of information that are ordered in a predetermined order. In an aspect, the predetermined order is a random order. In an aspect, L comprises bits of information comprising a key for decoding an order of the plurality of ordered probe reagent sets. In an aspect, the at least K bits of information comprise information about the absence of a signal for one of the N distinct target analytes.

In an aspect, the detection label is a fluorescent label. In an aspect, the barcode probe and the barcode moiety each comprise an oligonucleotide sequence complementary to each other. In an aspect, the substrate and the substrate binding moiety each comprise an oligonucleotide sequence complementary to each other. In an aspect, the substrate binding moiety comprises biotin, and wherein the substrate comprises streptavidin. In certain aspects, the methods comprise the step of performing a denaturation reaction after the ligation step to remove the oligonucleotide comprising the target nucleotide sequence variant from the ligation product before binding the ligation reaction product to the substrate.

In certain embodiments, disclosed herein are methods of detecting at least one target nucleotide sequence variant suspected of being present in a sample, comprising distributing a sample comprising a plurality of oligonucleotides suspected of comprising at least one target nucleotide sequence variant at a locus on a substrate so that they bind to the substrate at spatially separate regions of the substrate; carrying out on the oligonucleotides bound to the substrate a target nucleotide sequence variant identification assay comprising performing M number of detection cycles for target nucleotide sequence variant identification, wherein M is at least two, each cycle comprising contacting the enriched nucleic acid sample bound to the substrate with an target nucleotide sequence variant binding probe that binds preferentially to the target nucleotide sequence variant at the locus, the variant binding probe comprising a detectable label; washing the surface of the substrate to remove unbound variant binding probes; detecting the identity and location of the detectable label on the substrate; and if the cycle number is less than M, performing a denaturation reaction to remove bound variant binding probes from the oligonucleotide bound to the substrate; and determining from the sequence of detectable labels at the location on the substrate the presence or absence of the target nucleotide sequence variant suspected of being present in the sample. In certain aspects, the methods comprise further carrying out a target identification assay on the oligonucleotides bound to the substrate, wherein the target identification assay comprises: contacting the enriched nucleic acid sample bound to the substrate with a locus binding probe that binds preferentially to the locus, but does not bind preferentially the target nucleotide sequence variant at the locus with respect to a different sequence variant at the locus, wherein the locus binding probe comprising a detectable label; washing the surface of the substrate to remove unbound locus binding probes; and detecting the identity and location of the detectable label on the substrate. In certain aspects, for at least one cycle, all probes that bind to the locus comprise the same detection marker regardless of the presence of a particular sequence variant. In certain aspects, the methods further comprise the step of determining the presence or absence of the locus at the spatially separate regions of the substrate using bits of information from the at least one cycle wherein all probes that bind to the locus comprise the same detection marker. In certain aspects, the sample comprising the plurality of oligonucleotides is enriched to increase the proportion of oligonucleotides suspected of comprising at least one target nucleotide sequence variant at a locus as compared to an original sample.

In an embodiment, the specification describes methods of identifying at least one target oligonucleotide sequence variant suspected of being present in a sample, comprising distributing a sample on a substrate such that the plurality of oligonucleotides bind to the substrate at spatially separate regions of the substrate, wherein the oligonucleotides are suspected of comprising at least one target oligonucleotide sequence variant of a plurality of sequence variants at one of a plurality of target loci; carrying out on the oligonucleotides bound to the substrate a target oligonucleotide sequence variant identification assay for identifying at least one of N nucleotide sequence variants, wherein the assay comprises: providing at least M sets of sequence variant probes for performing at least M cycles of the assay, each set comprising sequence variant probes capable of binding preferentially to a single locus comprising one or more of the N nucleotide sequence variants, wherein each of the sequence variant probes comprise a detection label for generating K bits of information for the corresponding cycle; wherein for at least 2 of the M cycles, the sequence variant probe set comprises N sequence variant probes each capable of binding preferentially to a corresponding single one of the N nucleotide sequence variants; and performing at least M detection cycles to generate a signal detection sequence at the spatially separate regions of the substrate bound to the oligonucleotides, wherein M is at least 2, each cycle comprising contacting the oligonucleotides bound to the substrate with the sequence variant probe set corresponding with the cycle; washing the surface of the substrate to remove unbound sequence variant probes; detecting the identity and location of the detection label on the substrate to generate K bits of information at each of the spatially separate regions for the cycle; and if the cycle number is less than M, performing a denaturation reaction to remove bound sequence variant probes from the bound oligonucleotides; and determining from the at least M detection cycles L total bits of information, wherein the L equals the sum of the K bits of information generated at each of the M detection cycles, wherein L>log₂ (N), and wherein the L bits of information are used to identify one or more of the N oligonucleotide sequence variants. In certain aspects, K varies between two or more cycles. In certain aspects, the oligonucleotide sequence variant probe sets for cycles 1 through X are capable of identifying the locus, but not the sequence variant, and wherein X<M. In an aspect, the oligonucleotide sequence variant probe sets for cycles 1 through X comprise N sequence variant probes each capable of binding preferentially to a corresponding single one of the N nucleotide sequence variants, and wherein each probe that binds preferentially to a sequence variant at a particular target locus comprises the same detection marker as other sequence variants at the particular target locus for a particular cycle. In an aspect, the oligonucleotide sequence variant probe sets for cycles 1 through X comprises a plurality of sequence variant probes that bind preferentially to a target locus, but does not bind preferentially to a sequence variant at the target locus. In certain aspects of the methods, X is 1. In certain aspects, the oligonucleotide sequence variant probe sets for cycles (X+1) through M comprises the N sequence variant probes each capable of binding preferentially to a corresponding single one of the N nucleotide sequence variants. In an aspect, the oligonucleotide sequence variant probe sets for cycles (X+1) through M each comprise the same number of detection markers. In an aspect, the oligonucleotide sequence variant probe sets for all cycles comprise N sequence variant probes each capable of binding preferentially to a corresponding single one of the N nucleotide sequence variants. In certain aspects, the oligonucleotide sequence variant probe sets for all cycles comprise the same number of detection markers for generating K total bits of information at each cycle, and wherein L=K×M. In an aspect, the at least one of the N variant probes has a cross-reactivity with non-target sequence variant at the same loci of greater than 2%, 5%, 10%, 15%, 20%, or 25%. In an aspect, L is sufficient to reduce a false positive detection error rate from a single binding cycle to less than 1 in 10⁵, less than 1 in 10⁶, less than 1 in 10⁷, less than 1 in 10⁸, or less than 1 in 10⁹. In an aspect, at least one of the N oligonucleotide sequence variants bound to the substrate does not bind to a corresponding oligonucleotide sequence variant probe for at least 10%, at least 20%, at least 30%, or at least 40% of cycles wherein the probe set comprises the corresponding oligonucleotide sequence variant probe. In an aspect, L is sufficient to reduce a false negative error rate from a single cycle for at least one of the N oligonucleotide sequence variants to less than 0.1%, less than 0.01%, or less than 0.001% of the false negative error rate from a single cycle. In an aspect, L is a function of the average non-binding rate and the false binding rate of the variant probe set to the corresponding N oligonucleotide sequence variants. In an aspect, the assay determines a quantity of the one or more N nucleotide sequence variants. In an aspect, the target locus comprises a portion of a gene. In an aspect, the portion of a gene is a coding region. In an aspect, the oligonucleotide sequence variant is an allele. In an aspect, the allele comprises a mutation. In an aspect, the mutation is a deletion, a replacement, or an insertion. In an aspect, the mutation is a single nucleotide polymorphism. In an aspect, the target locus comprises at least two sequence variants. In an aspect, providing the enriched nucleic acid sample comprises contacting a sample comprising RNA with a reverse transcriptase enzyme. In an aspect, L comprises bits of information that are ordered in a predetermined order. In an aspect, the predetermined order is a random order. In an aspect, the L comprises bits of information comprising a key for decoding an order of the plurality of ordered probe reagent sets. In an aspect, the at least K bits of information comprise information about the absence of a signal for one of the N distinct target analytes. In an aspect, the detection label is a fluorescent label. In certain aspects, the sequence variant or locus-specific probe comprises PNA or LNA.

In certain embodiments, described herein are methods of detecting at least one target nucleotide sequence variant suspected of being present in a sample, comprising distributing a plurality of oligonucleotides on a substrate so that the plurality of oligonucleotides bind to the substrate at spatially separate regions, wherein the plurality of oligonucleotides are suspected of comprising the at least one target nucleotide sequence variant at least one of a plurality of loci; carrying out on the substrate a target nucleotide sequence variant identification assay, wherein the sequence variant identification assay comprises performing at least M detection cycles to generate a signal detection sequence, wherein M is at least two, each cycle comprising contacting the substrate with a set of primers each capable of binding preferentially to an oligonucleotide sequence immediately 5′ or 3′ to the location of one of the at least one target sequence variants, thereby forming a hybridized primer/oligonucleotide bound to the substrate when the at least one target sequence variant is bound to the substrate; contacting the substrate with reagents for performing a single nucleotide extension reaction, the reagents comprising at least one nucleotide comprising a detectable label and a terminator; exposing the substrate to conditions that promote a single nucleotide extension reaction at the 3′ terminus of the primer; washing the surface of the substrate to remove unbound nucleotides; detecting the identity and location of the detectable label on the substrate; and if the cycle number is less than M, performing a denaturation reaction to remove the primers bound to the oligonucleotides; and determining from the sequence of detectable labels for each cycle at a location on the substrate the presence or absence of the target nucleotide sequence variant suspected of being present in the sample. In an aspect, the detection label is a fluorescent label. In certain aspects, the nucleotide comprising a terminator is a ddNTP. In certain aspects, the nucleotides comprise any of ddATP, ddGTP, ddCTP, and ddTTP. In certain aspects, each cycle comprises addition of only one type of a nucleotide selected from the group consisting of: a nucleotide comprising adenosine, a nucleotide comprising guanine, a nucleotide comprising thymine, and a nucleotide comprising cytosine. In an aspect, the nucleotide extension reaction at each cycle comprises addition of all nucleotides comprising adenosine, guanine, thymine, and cytosine. In an aspect, detectable label corresponds to a unique nucleotide identity. In an aspect, the single base extension reaction is performed with a set of reagents comprising 4 distinctly labeled ddNTP, wherein each distinctly labeled ddNTP is bound to a distinct fluorophore. In an aspect, the plurality of oligonucleotides bound to the substrate comprises the + and − strand at the locus, wherein the target single nucleotide variant identification assay is redundantly performed on both the + and − strand. In certain aspects, the target nucleotide sequence variant is a mutation. In certain aspects, the mutation is an insertion, a deletion, a replacement, or a rearrangement. In an aspect, the target nucleotide sequence variant is a single nucleotide variant. In an aspect, the single nucleotide variant is a single nucleotide polymorphism. In an aspect, the target nucleotide sequence variant is an allelic variant. In an aspect, the nucleic acid sample is enriched. In certain aspects, the enrichment comprises contacting a sample comprising RNA with a reverse transcriptase enzyme to generate the enriched nucleic acid sample. In an aspect, the method further comprises contacting the oligonucleotides bound to the substrate with a locus specific probe that binds preferentially to a specific locus comprising any of the single nucleotide variants at the locus.

In an embodiment, the application describes methods of identifying at least one target single nucleotide variant suspected of being present in a sample, comprising distributing a nucleic acid sample comprising a plurality of oligonucleotides suspected of comprising at least one target single nucleotide variant of a plurality of single nucleotide variants at least one of a plurality of loci on a substrate such that the plurality of oligonucleotides bind to the substrate at spatially separate regions of the substrate; carrying out on the oligonucleotides bound to the substrate a target single nucleotide variant identification assay for identifying at least one of N single nucleotide variants at least one of a plurality of loci, the assay comprising providing a set of primers for each locus comprising at least one of the N single nucleotide variants, each of the set of primers capable of hybridizing to an oligonucleotide sequence immediately 5′ or 3′ to one of the N single nucleotide variants; preforming at least M detection cycles to generate a signal detection sequence at the spatially separate regions of the substrate bound to the oligonucleotides, wherein M is at least 2, each cycle comprising contacting the oligonucleotides bound to the substrate with the set of primers for each locus, thereby hybridizing the each of the sets of primers to the corresponding oligonucleotide sequence immediately 5′ or 3′ to the single nucleotide variant at the locus; contacting the oligonucleotides hybridized to the primers with a set of nucleotides for generating K bits of information for the corresponding cycle, the nucleotides comprising a terminator and a detectable label, and reagents for performing a single nucleotide extension reaction, each nucleotide comprising detectable label; exposing the substrate surface to conditions to promote a single nucleotide extension reaction; washing the surface of the substrate to remove unbound nucleotides; detecting the identity and location of the detection label on the substrate to generate K bits of information at each of the spatially separate regions for the cycle; and if the cycle number is less than M, performing a denaturation reaction to remove the primers bound to the oligonucleotides; and determining from the at least M detection cycles L total bits of information, wherein the L equals the sum of the K bits of information generated at each of the M detection cycles, wherein L>log₂ (N), and wherein the L bits of information are used to identify one or more of the N oligonucleotide sequence variants. In certain aspects, K varies between two or more cycles. In certain other aspects, K is constant for all cycles, and wherein L=K×M. In an aspect, the methods further comprise contacting the oligonucleotides bound to the substrate with a locus specific probe that binds preferentially to a specific locus comprising any of the single nucleotide variants at the locus. In certain aspects, the methods further comprise carrying out on the oligonucleotides bound to the substrate a locus identification assay comprising performing Q number of detection cycles for locus identification, wherein Q is at least two, each cycle comprising contacting the oligonucleotides bound to the substrate with a locus binding probe that binds preferentially to the locus, the locus binding probe comprising a detectable label; washing the surface of the substrate to remove unbound locus binding probes; detecting the identity and location of the detectable label on the substrate; and if the cycle number is less than Q, performing a denaturation reaction to remove bound allele binding probes from the oligonucleotide bound to the substrate; and determining from the sequence of detectable labels at the location on the substrate the presence or absence of the allele suspected of being present in the sample. In certain aspects, at least one of the primers binds non-specifically to an off target sequence as compared to the target sequence at a frequency of greater than 1%, 2%, 5%, 10%, 15%, 20%, or 25%. In an aspect, L is sufficient to reduce a false positive detection error rate from a single binding cycle to less than 1 in 10⁵, less than 1 in 10⁶, less than 1 in 10⁷, less than 1 in 10⁸, or less than 1 in 10⁹. In certain aspects, at least one of the oligonucleotides comprising one of the N single nucleotide variants bound to the substrate does not bind to a corresponding primer for at least 10%, at least 20%, at least 30%, or at least 40% of the M cycles. In an aspect, L is sufficient to reduce a false negative error rate of detection of at least one of N oligonucleotide sequence variants to less than 0.1%, less than 0.01%, or less than 0.001%. In an aspect, the assay determines a quantity of the one or more N single nucleotide variants. In certain aspects, N is at least 10, at least 20, at least 30, at least 40, at least 50, at least at least 100, at least 200, at least 500, or at least 1,000. In certain aspects, the limit of detection of the N nucleotide variants at the loci is less than 0.1% or less than 0.01%. In an aspect, the single nucleotide variant is a single nucleotide polymorphism. In certain aspects, the single nucleotide variant is an insertion, a deletion, or a replacement. In an aspect, the target locus comprises a portion of a gene. In an aspect, the portion of a gene is a coding region. In an aspect, the nucleic acid sample is enriched. In certain aspects, the enrichment comprises contacting a sample comprising RNA with a reverse transcriptase enzyme to generate the enriched nucleic acid sample. In an aspect, L comprises bits of information that are ordered in a predetermined order. In an aspect, the predetermined order is a random order. In an aspect, L comprises bits of information comprising a key for decoding an order of the plurality of ordered probe reagent sets. In an aspect, the at least K bits of information comprise information about the absence of a signal for one of the N distinct target analytes. In an aspect, the detection label is a fluorescent label. In an aspect, the nucleotide comprising a terminator is a ddNTP. In an aspect, the nucleotides comprise any of ddATP, ddGTP, ddCTP, and ddTTP. In an aspect, each cycle comprises addition of only one type of a nucleotide selected from the group consisting of: a nucleotide comprising adenosine, a nucleotide comprising guanine, a nucleotide comprising thymine, and a nucleotide comprising cytosine. In an aspect, the nucleotide extension reaction at each cycle comprises addition of all nucleotides comprising adenosine, guanine, thymine, and cytosine. In an aspect, the detectable label corresponds to a unique nucleotide identity. In an aspect, the single base extension reaction is performed with a set of reagents comprising 4 distinct labeled ddNTP, wherein each distinct labeled ddNTP is bound to a distinct fluorophore. In certain aspects, the plurality of oligonucleotides bound to the substrate comprises the + and strand at the locus, wherein the target single nucleotide variant identification assay is redundantly performed on both the + and − strand.

In an embodiment, described herein are methods of identifying at least one target nucleotide sequence variant suspected of being present in a sample, comprising providing an amplification reaction product of a sequence variant-specific amplification reaction performed on the sample, wherein the amplification reaction product comprises a plurality of oligonucleotides each comprising a substrate binding moiety and a barcode moiety; distributing the amplification reaction product on a substrate such that individual oligonucleotides bind to the substrate via the substrate binding moiety at spatially separate regions of the substrate; carrying out on the substrate a target nucleotide sequence variant identification assay, wherein the sequence variant identification assay comprises performing at least M detection cycles to generate a signal detection sequence, wherein M is at least two, each cycle comprising contacting the amplification reaction product with a barcode probe comprising a detection label, wherein the barcode probe binds to the barcode moiety when it is present on the substrate; washing the surface of the substrate to remove unbound barcode probes; detecting the identity and location of the detection label on the substrate; and if the cycle number is less than M, removing the barcode probe from the barcode moiety; and analyzing the signal detection sequence generated by the M cycles at the spatially separate locations on the substrate to determine the presence or absence of the at least one target nucleotide sequence variant of interest. In an aspect, the method comprises providing the amplification reaction product comprises carrying out the sequence variant-specific amplification reaction on the sample. In an aspect, the sample is an enriched nucleic acid sample suspected of comprising at least one target nucleotide sequence variant of a plurality of sequence variants at one of a plurality of target loci. In an aspect, the enriched nucleic acid sample is enriched by performing a reverse transcription reaction on a sample comprising RNA. In an aspect, the method comprises carrying out the sequence variant-specific amplification reaction on the sample comprises: providing a plurality of oligonucleotide primer sets, each set comprising a pair of oligonucleotide primers for amplifying a locus suspected of comprising the oligonucleotide sequence variant, the primer pair comprising a first oligonucleotide primer capable of specifically hybridizing to one of a plurality of nucleotide sequence variants at a target locus, wherein the primer is bound to the barcode moiety; a second oligonucleotide primer capable of specifically hybridizing to the target locus at a region upstream or downstream from the sequence variant, wherein the second oligonucleotide primer is bound to a substrate binding moiety; contacting the sample with the plurality of oligonucleotide primer sets and amplification reagents to perform the sequence variant-specific amplification reaction, thereby generating the amplification reaction product.

In an embodiment, described herein are methods of identifying at least one target nucleotide sequence variant suspected of being present in a sample, comprising providing an amplification reaction product of a sequence variant-specific amplification reaction performed on the sample, wherein the amplification reaction product comprises a plurality of oligonucleotides each comprising a substrate binding moiety and a barcode moiety; distributing the amplification reaction product on a substrate such that individual oligonucleotides bind to the substrate via the substrate binding moiety at spatially separate regions of the substrate; carrying out on the substrate a target nucleotide variant identification assay for identifying at least one of N nucleotide sequence variants, wherein the assay comprises: providing at least M sets of barcode probes for performing at least M cycles of the assay, each set comprising N unique barcode binding moieties capable of binding preferentially to a corresponding one of the N barcode moieties for generating K bits of information per cycle; performing at least M detection cycles to generate a signal detection sequence at a plurality of the spatially separate regions on the substrate, wherein M is at least one, each cycle comprising contacting the substrate bound to the allele specific amplification reaction products with the barcode probe set corresponding with the cycle number; washing the surface of the substrate to remove unbound barcode probes; detecting the presence or absence of a plurality of signals from the spatially separate regions of the substrate; and if the cycle number is less than M, performing a denaturation reaction to remove the barcode probe from the barcode moiety; and determining from the at least M detection cycles L total bits of information, wherein K×M=L and L>log₂ (N), and wherein the L bits of information are used to identify one or more of the N nucleotide sequence variants. In an aspect, the method comprises providing the amplification reaction product comprises carrying out the sequence variant-specific amplification reaction on the sample.

In an aspect, the sample is an enriched nucleic acid sample suspected of comprising at least one target nucleotide sequence variant of a plurality of sequence variants at one of a plurality of target loci. In an aspect, the enriched nucleic acid sample is enriched by performing a reverse transcription reaction on a sample comprising RNA. In certain aspects, carrying out the sequence variant-specific amplification reaction on the sample comprises: providing N oligonucleotide primer sets, each set comprising a first oligonucleotide primer capable of specifically hybridizing to one of a plurality of nucleotide sequence variants at a target locus, wherein the primer is bound to the barcode moiety; a second oligonucleotide primer capable of specifically hybridizing to the target locus at a region upstream or downstream from the sequence variant, wherein the second oligonucleotide primer is bound to a substrate binding moiety; contacting the sample with the N oligonucleotide probe sets and amplification reagents to perform an allele specific amplification reaction, thereby generating the amplification reaction product.

### BRIEF DESCRIPTION OF THE SEVERAL VIEWS OF THE DRAWINGS

These and other features, aspects, and advantages of the present invention will become better understood with regard to the following description, and accompanying drawings, where

FIG. 1 illustrates a locus-specific oligonucleotide (LSO) detection via ligation protocol including detection and error correction steps, according to an embodiment of the invention.

FIG. 2 diagrams allele specific probes with a barcode moiety and locus specific probes with a substrate binding moiety bound to allele and ligation product formed according to an embodiment of the invention.

FIG. 3 illustrates a ligation product comprising a substrate binding moiety, barcode probe and capture moiety according to an embodiment of the invention.

FIG. 4 shows the genotyping results for detection of the EGFR allele harboring the mutation L858R.

FIG. 5 shows the genotyping results for detection of the BRAF allele harboring the V600E mutation.

FIG. 6 shows the genotyping results for detection of the EGFR allele harboring the mutation T790M.

FIG. 7 shows the genotyping results for detection of the EGFR allele harboring the mutation L858R by locus-specific oligonucleotide detection via ligation and detection of mutant targets at a 0.5% minor allele frequency.

FIG. 8 illustrates samples and oligonucleotides bound to a substrate in a randomly ordered format according to an embodiment of the invention.

FIG. 9 is a diagram of a protocol for detection of a target bound to a substrate by hybridization of allele-specific probes including detection and error correction steps, according to an embodiment of the invention.

FIG. 10 shows locus-specific probes bound to substrate, alleles and allele-specific probes bound to substrate with different detection moieties, according to an embodiment of the invention.

FIG. 11 shows the results of detection of Epidermal Growth Factor Receptor (EGFR) Exon 19 deletion mutations by hybridization and detection of allele-specific probes.

FIG. 12 is a diagram of a protocol for detection of single nucleotide polymorphisms comprising single nucleotide extension and including detection and error correction steps, according to an embodiment of the invention.

FIG. 13 is a diagram of a locus-specific oligonucleotide (LSO) adjacent to SNP on allele and extension products with labeled ddNTPs, according to an embodiment of the invention.

FIG. 14 shows the genotyping results using detection by single base extension with labeled ddNTPs of a locus-specific oligonucleotide adjacent to SNPs of the EGFR gene.

FIG. 15 is a diagram of a protocol comprising allele-specific PCR including detection and error correction, according to an embodiment of the invention.

FIG. 16 illustrates allele-specific oligos with barcodes and common primers with substrate binding moiety bound to alleles, according to an embodiment of the invention.

FIG. 17 illustrates amplification products with barcodes bound to substrate and barcode probes bound to amplification products, according to an embodiment of the invention.

### DETAILED DESCRIPTION

Throughout this specification, unless specifically stated otherwise or the context requires otherwise, reference to a single step, feature, composition of matter, group of steps or group of features or compositions of matter shall be taken to encompass one and a plurality (i.e., one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of the steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

### Advantages and Utility

As provided herein, several embodiments of the invention are useful for the simultaneous detection of the presence or absence of multiple nucleotide sequence variants, such as genetic polymorphisms, with increased accuracy over prior approaches. Also described herein are methods that allow for highly sensitive detection of a plurality of sequence variants of many loci in a single assay.

### Selected Definitions

Terms used in the claims and specification are defined as set forth below unless otherwise specified.

The term “sample” as used herein refers to a specimen, culture, or collection from a biological material. Samples may be derived from or taken from a mammal, including, but not limited to, humans, monkey, rat, or mice. Samples may be include materials such as, but not limited to, cultures, blood, tissue, formalin-fixed paraffin embedded (FFPE) tissue, saliva, hair, feces, urine, and the like. These examples are not to be construed as limiting the sample types applicable to the present invention.

The term “enriched nucleic acid sample” as used herein refers to a sample comprising nucleic acid of interest that has been processed to remove unwanted substances from the sample. The enriched nucleic acid sample can be generated by any processes to remove non-nucleic acid biological material such as, but not limited to, carbohydrates, proteins, and/or lipids. The enriched nucleic acid sample can be generated by remove unwanted nucleic acids and/or amplifying nucleic acids of interest. Any process to remove unwanted substances can be employed, including, but not limited to, separation on the basis of electrical charge (e.g., electrophoretic separation, ion-exchange chromatography), size (e.g., filtration, size-exclusion chromatography, molecular sieving, etc.), density (e.g., regular or gradient centrifugation), Svedberg constant (e.g., sedimentation with or without external force, etc.). Generation of an enriched nucleic acid sample may comprise using oligonucleotides that anneal to target nucleic acids. In certain embodiments, the enriched nucleic acid sample can be generated using a plurality of distinct oligonucleotides and/or can be generated using oligonucleotides that bind to nucleic acids of interest non-specifically. For example, mRNAs can be enriched by oligonucleotides that bind to poly(A) sequences on the 3′ terminus and/or complementary DNAs (cDNAs) can be enriched by oligonucleotides that bind to Poly(T) sequences. The enriched nucleic acid may be enriched by performing a reverse transcription reaction to produce cDNA from RNA. The oligonucleotides used to generate enriched nucleic acid sequences can comprise tags (e.g., fluorescent molecules, chemiluminescent molecules, etc.), moieties for binding to substrates and/or moieties used for purification of nucleic acids of interest (e.g., affinity tags such as biotin, etc.). The enriched nucleic acid sample may comprise nucleic acid from a single origin or a plurality of origins (e.g., nucleic acid derived from multiple patients or individuals).

The term “target analyte” or “analyte” as used herein refers to a molecule, compound, substance or component that is to be identified, quantified, and otherwise characterized. A target analyte can comprise by way of example, but not limitation to, an atom, a compound, a molecule (of any molecular size), a polypeptide, a protein (folded or unfolded), an oligonucleotide molecule (RNA, cDNA, or DNA), a fragment thereof, a modified molecule thereof, such as a modified nucleic acid, or a combination thereof. In an embodiment, a target analyte polypeptide or protein is about nine amino acids in length. Generally, a target analyte can be at any of a wide range of concentrations (e.g., from the mg/mL to ag/mL range), in any volume of solution (e.g., as low as the picoliter range). For example, samples of blood, serum, formalin-fixed paraffin embedded (FFPE) tissue, saliva, or urine could contain various target analytes. The target analytes are recognized by probes, which are used to identify and quantify the target analytes using electrical or optical detection methods.

The term, “complementary” as used herein refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence. The term “complementary” is used to indicate a sufficient degree of complementarity or precise pairing such that stable and specific binding occurs between nucleic acid sequences e.g., between a probe sequence and the target sequence (e.g., nucleotide sequence variant) of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4, 5, 6, 7, 8, 9 or more mismatches.

The term, “oligonucleotide” as used herein refers to a nucleic acid that is between 100 and 10 nucleotides in length, between 50 and 10 nucleotides in length, between 30 and 10 nucleotides in length, between 25 and 10 nucleotides in length, between 20 and 10 nucleotides in length, between 15 and 10 nucleotides in length. Oligonucleotides can comprise non-nucleic acid substances (e.g., substances used as tags, etc.)

The term “locus” as used herein refers to the nucleotide sequence position on a chromosome. A locus may indicate or refer to a general position that includes a region surrounding a more specific location on a chromosome. The region surrounding the more specific region may be as long as 10 kilobases or less, 5 kilobases or less, 1 kilobase or less, 100 bases or less or 10 bases or less. A locus may be either the positive strand, the negative strand or both the positive and negative strands of DNA. A locus can comprise the portion of a gene, a coding region or a non-coding region.

The term “nucleotide sequence variant” or “sequence variant” as used herein refers to any nucleotide sequence that has at least one nucleotide base difference in sequence than another sequence at the same locus on the genome or another sequence corresponding to or derived from the same locus, such as mRNA sequences or cDNA sequences derived from mRNAs. Nucleotide sequence variants are not limited to coding regions of genes and may comprise any oligonucleotide sequence with similar sequence to another oligonucleotide of interest. The at least one base difference in sequence may comprise one or more nucleotide additions, insertions, deletions, replacements, rearrangements and/or other mutations. Sequence variants comprise alleles, single nucleotide polymorphisms, mutations, low incidence mutations, etc.

The term “allele” as used herein refers to one of at least two alternative forms of a nucleotide sequence at the same locus on the genome. Alleles can be naturally found in a biological material or may be non-natural or generated by sequence alteration of a nucleic acid sequence.

The term “allelic variant” as used herein refers to a nucleic acid that differs in sequence by at least one nucleotide between two or more alleles for a given locus.

The term “constant region” as used herein, refers to a sequence or region of nucleic acid that has an identical sequence to at least one other variant sequence.

The term, “probe” as used herein refers to a molecule that is capable of binding to other molecules (e.g., oligonucleotides comprising DNA or RNA, polypeptides or full-length proteins, etc.). The probe comprises a structure or component that binds to the target analyte. In some embodiments, multiple probes may recognize different parts of the same target analyte. Examples of probes include, but are not limited to, an aptamer, an antibody, a polypeptide, an oligonucleotide (DNA, RNA), or any combination thereof. In certain aspects, probes comprise a detectable label or tag. In certain aspects, probes are modified for conjugation of a detection moiety or a substrate binding moiety. In certain aspects, oligonucleotide probes are modified with a peptide nucleic acid (PNA) or locked nucleic acid (LNA) to block binding of a label for optimization of detection methods to account for different binding activities of probes. Probes can have a cross-reactivity with non-target sequences. In certain aspects, probes has a cross-reactivity with non-target sequence variant of greater than 2%, 5%, 10%, 15%, 20%, 25%, 50% or 75%. In general, the affinity of an oligonucleotide probe to a target oligonucleotide sequence increases continuously with oligonucleotide length. In a preferred embodiment, oligonucleotide probes have a dissociation constant in the range of about 10⁻⁹ to 10⁻⁶ molar, in the range of 10⁻⁹ to 10⁻⁸ molar, in the range of 10⁻⁸ to 10⁻⁷ or the range of 10⁻⁷ to 10⁻⁶ molar.

The term “allele-specific probe” as used herein refers to a probe that has higher affinity or preferential binding affinity for one or more specific variants of a nucleotide sequence with respect to at least one other variant corresponding to the same locus. In general, the affinity of an oligonucleotide probe to a target oligonucleotide sequence increases continuously with oligonucleotide length. In a preferred embodiment, oligonucleotide probes have a dissociation constant in the range of about 10⁻⁹ to 10⁻⁶ molar, in the range of 10⁻⁹ to 10⁻⁸ molar, in the range of 10⁻⁸ to 10⁻⁷ or the range of 10⁻⁷ to 10⁻⁵ molar.

The term “locus-specific probe” as used herein refers to a probe that has affinity to a plurality of nucleotide sequence variants corresponding to a particular locus. In certain embodiments, the locus-specific probe does not have preferential affinity to a nucleotide sequence variant with respect to at least one different sequence variant at the same locus. In certain embodiments, the locus-specific probe binds to a constant region at a particular locus of interest. In general, the affinity of an oligonucleotide probe to a target oligonucleotide sequence increases continuously with oligonucleotide length. In a preferred embodiment, oligonucleotide probes have a dissociation constant in the range of about 10′ to 10-6 molar, in the range of 10-9 to 10-8 molar, in the range of 10-8 to 10-7 or the range of 10-7 to10-6 molar.

The term “sequence variant probe”, “target nucleotide sequence variant binding probe”, “variant binding probe” or “variant probe” as used herein refers to a probe capable of binding preferentially to a corresponding single one of a plurality of nucleotide sequence variants. In certain aspects, the variant probes have a cross-reactivity with non-target sequence variant at the same loci of greater than 2%, 5%, 10%, 15%, 20%, or 25%. In general, the affinity of an oligonucleotide probe to a target oligonucleotide sequence increases continuously with oligonucleotide length. In a preferred embodiment, oligonucleotide probes have a dissociation constant in the range of about 10″″9 to 10-6 molar, in the range of 10-9 to 10-8 molar, in the range of 10-8 to 10-7 or the range of 0-1 to 10-6 molar.

The term “barcode” or “barcode moiety” as used herein refers to a molecular substance that can be used to identify one or more nucleic acids from a plurality of nucleic acids. In preferred embodiments, the barcode is a nucleotide sequence can identify one or more nucleic acids. In certain embodiments, the barcode is a nucleotide sequence between 30 and 20 nucleotides in length, between 25 and 20 nucleotides in length, between 20 and 15 nucleotides in length, between 15 and 10 nucleotides in length or between 10 and 5 nucleotides in length. In certain embodiments, the barcode is DNA. Barcodes can further comprise non-nucleic acid substances (e.g., substances used as tags, etc.).

The term “barcode probe” as used herein refers to an oligonucleotide probe that can hybridize to one more barcode moieties under high or low stringency conditions. In certain aspects, barcode probes are complementary or partially complementary to one or more barcode moieties.

The term “substrate” as used herein refers to any solid or semi-solid support used for adhering to analysts (i.e., nucleic acids) of interest. A substrate can be made of any suitable material, such as, but not limited to, glass, metal, plastic, membranes, a gel, silicon, carbohydrate surfaces, etc. A substrate can be flat two-dimensional surfaces or three-dimensional surfaces, such as micro-beads or micro-spheres. Substrates can be coated or treated with substances to alter the binding characteristics of the substrate to analytes of interest (e.g., glass or silicon surfaces treated with amino silane and glass surfaces treated with epoxy silane-derivatized or isothiocyanate). Substrates may also be coated or bound to adapters (such as oligonucleotides) that specifically bind targets of interest (e.g., the enriched nucleic acid, ligation products and amplification products). Adapters, including oligonucleotide adapters coated on substrates can be used to generate addressable arrays wherein the location of the oligonucleotide adapters at distinct regions on the substrate correspond to specific targets.

The term “substrate binding moiety” as used herein refers to any molecule or substance that is used for the binding or conjugation of an analyte comprising a nucleic acid molecule to the substrate or solid support.

The term “primer” as used herein refers to an oligonucleotide used for an extension or amplification reaction that hybridizes to a nucleic acid of interest.

The term “label”, “detectable label” or “detection label” as used herein refers to a molecule capable of detecting a target analyte. The label can be, but is not limited to, a fluorescent label and/or an oligonucleotide sequence. The label can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, and the like. The tag can be directly or indirectly bound to, hybridizes to, conjugated to, or covalently linked to a probe.

The term “+strand”, “plus strand” or “sense strand” as used herein refers to the nucleotide sequence of a DNA that directs the synthesis of protein when in RNA form (i.e., the single strand of DNA of a double stranded DNA gene that is not used as the template for RNA Polymerases during transcription of the gene to messenger RNA).

The term “−strand” or minus strand” or “anti-sense strand” as used herein refers to a nucleotide sequence that is complementary to the +strand, positive strand or sense strand. (i.e., the single strand of DNA of a double stranded DNA gene that is used as the template for RNA Polymerases during transcription of the gene to messenger RNA).

A “pass” in a detection assay as used herein refers to a process where a plurality of probes are introduced to the bound analytes, selective binding occurs between the probes and distinct target analytes, and a plurality of signals are detected from the probes. A pass includes introduction of a set of antibodies that bind specifically to a target analyte. There can be multiple passes of different sets of probes before the substrate is stripped of all probes.

A “cycle” is defined by completion of one or more passes and stripping of the probes from the substrate, if needed for subsequent cycles. Subsequent cycles of one or more passes per cycle can be performed. Multiple cycles can be performed on a single substrate or sample. For proteins, multiple cycles will require that the probe removal (stripping) conditions either maintain proteins folded in their proper configuration, or that the probes used are chosen to bind to peptide sequences so that the binding efficiency is independent of the protein fold configuration.

The term “bit” as used herein refers to a basic unit of information in computing and digital communications. A bit can have only one of two values. The most common representations of these values are 0 and 1. The term bit is a contraction of binary digit. In one example, a system that uses 4 bits of information can create 16 different values. All single digit hexadecimal numbers can be written with 4 bits. Binary-coded decimal is a digital encoding method for numbers using decimal notation, with each decimal digit represented by four bits. In another example, a calculation using 8 bits, there are 28 (or 256) possible values.

The term “hybridizing” as used herein refers to the annealing of a nucleic acid molecule to another nucleic acid molecule through the formation of one or more hydrogen bonds (i.e., base pairing of complementary nucleotides by hydrogen bond formation). Nucleic acids may be hybridized under any conditions known and used in the art to efficiently anneal oligonucleotides to nucleic acids of interest. Oligonucleotides may be hybridized in conditions that vary significantly in stringency to compensate for probe binding activity with respect to target binding and off-target binding.

The term “extension” or “extension reaction” as used herein refers to generation of a single complementary copy of a nucleic acid sequence. In certain embodiments, extension reactions are performed as a result of an oligonucleotide probe hybridizing to a target nucleic acid sequence; wherein the probe is shorter than the target nucleotide sequence and a polymerase is used to synthesize and extend a nucleotide strand complementary to the target sequence from the 3′ terminus of the probe.

The term, “ligating” as used herein refers to covalently attaching polynucleotide sequences together to form a single sequence. This is typically performed by treatment with is ligase which catalyzes the formation of a phosphodiester bond between the 5′end of one sequence and the 3′ end of the other. However, in the context of the invention, the term “ligating” is also intended to encompass other methods of covalently attaching, such sequences, e.g., by chemical means.

The term “amplification” as used herein refers to synthesis of at least one additional nucleic acid molecule complementary to a template nucleic acid molecule to generate an increased abundance of a nucleic acid sequence and/or its complementary sequence. Amplification reactions include, but are not limited to, a polymerase chain reaction (PCR), a loop-mediated isothermal amplification (LAMP), a strand displacement amplification, a multiple displacement amplification, a recombinase polymerase amplification, a helicase dependent amplification and a rolling circle amplification.

The term “amplification reagents” as used herein refers to any substances or reagents added to mixture to facilitate an amplification of nucleic acid (i.e., oligonucleotide primers, polymerases, nucleotides, salts, buffers, etc.).

Abbreviations used in this application include the following: Complementary DNA (cDNA), polymerase chain reaction (PCR), oligonucleotide ligation assay (OLA), allele-specific PCR (AS-PCR), locus specific oligonucleotide (LSO), single-base extension (SBE), allele specific oligonucleotide (ASO) and 2′,3′ dideoxynucleotide (ddNTP).

It must be noted that, as used in the specification and the appended claims, the singular forms “a,” “an” and “the” include plural referents unless the context clearly dictates otherwise.

### General Description

(i) Overview of Methodology

Detection techniques for highly multiplexed single molecule identification and quantification of analytes using optical systems are disclosed. Analytes include, but are not limited to, nucleic acid, such as DNA and RNA molecules, with and without modifications. Techniques include complementary specific and non-specific probes for detailed characterization of analytes and highly multiplexed single molecule identification and quantification using probes. Probes can be conjugated to detection moieties or tags. Optical detection is accomplished by detection of fluorescent or luminescent tags, described in more detail below and in U.S. Patent publication US20150330974 A1, which is incorporated herein by reference in its entirety.

### Nucleotide Sequence Variants

Nucleotide sequence variants include any nucleotide sequence that has at least one nucleotide base difference in sequence compared to another sequence at the same locus on the genome, or compared to another sequence corresponding to or derived from the same locus, such as mRNA sequences or cDNA sequences derived from mRNAs. The at least one base difference in sequence may comprise one or more nucleotide additions, insertions, deletions, replacements, rearrangements and/or other mutations. Sequence variants comprise alleles, single nucleotide polymorphisms, mutations, low incidence mutations, etc. Nucleotide sequence variants are not limited to coding regions of genes and may comprise any oligonucleotide sequence with similar sequence to another oligonucleotide of interest.

(ii) Enrichment of a Nucleic Acid Samples

Removal of unwanted substances from the sample or reducing the complexity of a population of nucleic acids is performed prior to performing the methods described in the application. The enriched nucleic acid sample can be generated by any processes to remove non-nucleic acid biological material such as, but not limited to, carbohydrates, proteins, and/or lipids. In certain embodiments, extraction reagents may be used to produce an enriched nucleic acid sample. Examples of extraction agents for the extraction of nucleic acids comprise: phenol, chloroform, ethanol, methanol or other suitable methods for precipitating nucleic acids from mixtures of cellular debris following lysis of cells.

The enriched nucleic acid sample can be generated by remove unwanted nucleic acids and/or amplifying nucleic acids of interest. For example, DNA, such as genomic DNA can undergo an amplification step prior to performing the methods of the invention to produce an enriched nucleic acid sample. Nucleic acids can be amplified by any procedure known in the art including, a polymerase chain reaction (PCR), a loop-mediated isothermal amplification (LAMP), a strand displacement amplification, a multiple displacement amplification, a recombinase polymerase amplification, a helicase dependent amplification and a rolling circle amplification. The amplification may be performed to generate one or more copies of particular nucleic acids of interest (e.g., using specific primers that anneal to specific loci of interest) or may be performed non-specifically (e.g., using random or universal primers). Any process to separate and/or remove unwanted substances can be employed, including, but not limited to, separation on the basis of electrical charge (e.g., electrophoretic separation, ion-exchange chromatography), size (e.g., filtration, size-exclusion chromatography, molecular sieving, etc.), density (e.g., regular or gradient centrifugation), Svedberg constant (e.g., sedimentation with or without external force, etc.). In certain embodiments, manual separation is employed to enrich the nucleic acid of interest. In certain embodiments devices such as, centrifugation columns or microfluidic devices are used to enrich the nucleic acid. Generation of an enriched nucleic acid sample may comprise using oligonucleotides that anneal to target nucleic acids. In certain embodiments, the enriched nucleic acid sample can be generated using a plurality of distinct oligonucleotides and/or can be generated using oligonucleotides that bind to nucleic acids of interest non-specifically. For example, mRNAs can be enriched by oligonucleotides that bind to poly(A) sequences on the 3′ terminus of mRNAs and/or complementary DNA (cDNA) can be enriched by use of oligonucleotides that bind to Poly(T) sequences. In certain embodiments, reverse transcription using a reverse transcriptase is performed to generate cDNA. The oligonucleotides used to generate enriched nucleic acid sequences can comprise tags (e.g., fluorescent molecules, chemiluminescent molecules, etc.), moieties for binding to substrates and/or moieties used for purification of nucleic acids of interest (e.g., affinity tags such as biotin, etc.). In certain embodiments, the enrichment of nucleic acid may comprise use of antibodies that bind to specific chromatin binding proteins or other proteins bound either, directly or indirectly to DNA or RNA (for example use of antibodies for chromatin immunoprecipitation). In certain embodiments, the affinity tag or antibody is conjugated to a magnetic bead for magnetic separation. Enrichment can comprise use of a substrate or solid support to immobilize nucleic acids of interest. In certain embodiments, the enrichment process comprises an amplification step to generate increased abundance of nucleic acids of interest prior to performing the methods described herein. In certain embodiments, a microfluidic device can be employed (i.e., an electrophoretic microfluidic device), to enrich the nucleic acids of interest. Enriched nucleic acid samples may comprise nucleic acids from a single origin or from a plurality of origins (e.g., nucleic acids derived from more than one patient or individual). In certain embodiments, a particular target nucleotide sequence variant (e.g., a low frequency mutant allele) is enriched by blocking the detection (e.g., by incorporation of a PNA or LNA) of a more abundant (e.g., wild-type) nucleotide sequence.

Once the nucleic acid sample is enriched and/or purified, other treatments to the enriched nucleic acid sample may be performed, such as, but not limited to, fragmentation of the nucleic acid (e.g., by chemical or physical means), chemical crosslinking, amplification, conjugation of tags or detection markers and/or sequencing prior to performing the methods of the invention.

### Design, Complementarily and Hybridization of Probes

Probes described herein can be complementary to a target nucleotide sequence of interest. Oligonucleotide probes may be any length that allows efficient binding to a target sequence. In certain aspects probes are less than 200 nucleotides in length, less than 100 nucleotides in length, less than 80 nucleotides in length, less than 50 nucleotides in length, less than 40 nucleotides in length, less than 30 nucleotides in length or less than 20 nucleotides in length. The complementarity of the probes is a precise pairing such that stable and specific binding occurs between nucleic acid sequences e.g., between a probe sequence and the target sequence (e.g., nucleotide sequence variant) of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4, 5, 6, 7, 8, 9 or more mismatches. In certain aspects, the number of mismatches is 20% or less, 10% or less, 5% or less or 2% or less of the number of nucleotides present in the probe. In certain aspects, the probes are complementary to at least 18, at least 17, at least 16, at least 15, at least 14, at least 13, at least 12, at least 11, at least 1, at least 9, at least 8, at least 7, at least 6 or at least nucleotides of a target nucleotide sequence. In certain aspects, probes are complementary to one or more individual nucleotide sequence variants. In certain aspects, the probes do not bind to alternative sequences because of mismatches in sequences leading to loss of complementarity.

Probes may be hybridized to target sequences under any conditions known and used in the art to efficiently anneal oligonucleotide probes to nucleic acids of interest. Probes may be hybridized in conditions that vary significantly in stringency to compensate for probe binding activity with respect to target binding and off-target binding. Probe hybridization conditions can also vary depending on, for example, probe length, probe sequence (such as G+C content), concentration of nucleic acid present in the sample. Generally, more stringent conditions (such as higher temperature or use of buffers with detergents or denaturants and lower salt concentration) are used when probes are longer or have greater numbers of similar sequences present in the sample to reduce non-specific or off-target binding.

(iii) Design and Synthesis of Barcode Moieties

In certain embodiments, barcode moieties are used to identify a nucleic acid sequence. In certain aspects, the barcode determines the identity of a nucleotide sequence variant of interest. In certain aspects, the barcode determines an allele. In certain aspects, the barcode can determine the origin of a sample or nucleic acid sequence (e.g., such as the individual patient of origin of a nucleic acid sample derived from a patient). In certain aspects, oligonucleotide probes comprise a barcode moiety. In certain aspects, an oligonucleotide probe comprises more than one barcode moiety. In certain embodiments, the barcode is a nucleotide sequence between 30 and 20 nucleotides in length, between 25 and 20 nucleotides in length, between 20 and 15 nucleotides in length, between 15 and 10 nucleotides in length or between 10 and 5 nucleotides in length. In certain embodiments, the barcode is DNA. Barcode moieties can further comprise non-nucleic acid substances (e.g., substances used as tags, etc.).

Methods for the synthesis of barcode moieties include in certain embodiments, random addition of mixed bases during nucleic acid synthesis to produce a sequence that can be used to identify a specific oligonucleotide molecule through analysis of sequencing data. In certain embodiments, synthesis of barcode moieties comprises the controlled addition of bases to generate a known sequence. Barcode sequences can be verified by sequencing. In certain aspects, barcode moieties can be synthesized and extended using polymerase to attach the barcode moiety to oligonucleotides including oligonucleotide probes such as, nucleotide sequence variant probes, allele-specific probes or locus-specific probes. In other aspects, barcode sequences can be synthesized without probes and either ligated or annealed to the probes in a separate step.

(iv) Substrate Binding Moieties

Oligonucleotides described in the application can comprise substrate binding moieties. The nature of the substrate binding moieties will correspond to the type of substrate or solid support to be used for binding to the oligonucleotide. A substrate can be any solid or semi-solid support used for adhering to analysts (i.e., nucleic acids) of interest. A substrate can be made of any suitable material, such as, but not limited to, glass, metal, plastic, a gel, membranes, silicon, a carbohydrate surface, etc. Substrate binding moieties can be, for examples, modified nucleotides. The oligonucleotides can be modified by any suitable method known in the art for attachment of nucleic acid to substrates, for example, by conjugation to biotin, generating amine or thiol group modifications, covalently linked to a thioester or conjugated to a cholesterol-TEG. Modification of oligonucleotides to produce substrate binding moieties may occur at the 5′ terminus, 3′ terminus or at any position within the oligonucleotide. Linkers or spacers may be added between the terminus of the oligonucleotide and the substrate binding moiety. Substrate binding moieties may be bound directly or indirectly to the oligonucleotides.

The type of solid support chosen will be chosen based on the level of scattering and fluorescence background inherent in the support material and added chemical groups; the chemical stability and complexity of the construct; the amenability to chemical modification or derivatization; surface area; loading capacity and the degree of non-specific binding of the final product. Substrates can be prepared by treating glass or silicon surfaces, for example, with avidin for the binding to biotin-conjugated oligonucleotides. In another example, glass or silicon surfaces can be treated with an amino silane. Oligonucleotides modified with an NH2 group can be immobilized onto epoxy silane-derivatized or isothiocyanate coated glass slides. Succinylated oligonucleotides can be coupled to aminophenyl- or aminopropyl-derivatized glass slides by peptide bonds, and disulfide-modified oligonucleotides can be immobilized onto a mercaptosilanized glass support by a thiol/disulfide exchange reaction or through chemical cross-linkers. Amine-modified oligonucleotides can be reacted with carboxylate-modified micro-spheres with a carbodiimide, such as EDAC. Substrates may also be magnetic (such as magnetic microspheres) and bind to oligonucleotides conjugated or annealed to magnetic moieties.

(v) Labeled Probes

Described herein are methods comprising oligonucleotide probes. In certain embodiments, the methods comprise use of oligonucleotide probes comprising DNA. In certain embodiments, the probes are complementary to a target sequence suspected of being present in an enriched nucleic acid sample. In certain aspects, the target sequence is DNA. In certain other aspects, the target sequence is mRNA. In certain embodiments, the probes are complementary to a barcode sequence. In certain embodiments, the probe is complementary to one or more nucleotide sequence variants of interest. In certain embodiments, the probes are complementary to a constant region. In certain aspects, probes are complementary to a gene. In certain aspects, the probes are complementary to a coding-region or a non-coding region of a gene. Upon hybridization, probes may create a binding pair with a target of interest. The binding pair can be for example, a nucleotide sequence variant probe annealed to genomic DNA or other DNA (such as mitochondrial DNA or cDNA); a nucleotide sequence variant probe annealed to mRNA, a locus-specific probe annealed to genomic DNA or other DNA (such as mitochondrial DNA or cDNA); a locus-specific probe annealed to mRNA; a barcode probe annealed to barcode on genomic DNA or other DNA or a barcode probe annealed to a barcode on mRNA.

In some embodiments, the probe comprises a molecular tag for detection of the target analyte. Tags can be attached chemically or covalently to other regions of the probe. In some embodiments, the tags are fluorescent molecules. Fluorescent molecules can be fluorescent proteins or can be a reactive derivative of a fluorescent molecule known as a fluorophore. Fluorophores are fluorescent chemical compounds that emit light upon light excitation. In some embodiments, the fluorophore selectively binds to a specific region or functional group on the target molecule and can be attached chemically or biologically. Examples of fluorescent tags include, but are not limited to, green fluorescent protein (GFP), yellow fluorescent protein (YFP), red fluorescent protein (RFP), cyan fluorescent protein (CFP), fluorescein, fluorescein isothiocyanate (FITC), tetramethylrhodamine isothiocyanate (TRITC), cyanine (Cy3), phycoerythrin (R-PE) 5,6-carboxymethyl fluorescein, (5-carboxyfluorescein-N-hydroxysuccinimide ester), Texas red, nitrobenz-2-oxa-1,3-diazol-4-yl (NBD), coumarin, dansyl chloride, and rhodamine (5,6-tetramethyl rhodamine).

(vi) Methods for Optical Detection of Analytes

For optical detection of the analytes, in certain embodiments, the analytes are spatially separated on the solid substrate, so that there is no overlap of fluorescent signals. For a random array, multiple pixels are needed for each fluorescent spot. The number of pixels can be as few as 1 and as many as hundreds of pixels per spot. It is expected that the optimal amount of pixels per fluorescent spot is between 5 and 20 pixels. In one example, an imaging system has 224 nm pixels. For a system with 10 pixels per fluorescent spot on average, there is a surface density of 2 fluorescent pixels/μm2. This does not mean that the surface density of the analytes needs to be this low. If probes are only chosen for low abundance analytes, then the amount of analytes on the surface may be much higher. For instance, if there are, on average, 20,000 analytes per μm2 on the surface, and probes are chosen only for the rarest 0.01% (as an integrated sum) analytes, then the fluorescent analyte surface density will be 2 fluorescent pixels/μ2. In another embodiment, the imaging system has 163 nm pixels. In another embodiment, the imaging system has 224 nm pixels. In a preferred embodiment, the imaging system has 325 nm pixels. In other embodiments, the imaging system has as large as 500 nm pixels.

Optical detection methods can be used to quantify and identify a large number of analytes simultaneously in a sample. In an embodiment, optical detection of fluorescently-tagged single molecules can be achieved by frequency-modulated absorption and laser-induced fluorescence. Fluorescence can be more sensitive because it is intrinsically amplified as each fluorophore emits thousands to perhaps a million photons before it is photobleached. Fluorescence emission usually occurs in a four-step cycle: 1) electronic transition from the ground-electronic state to an excited-electronic state, the rate of which is a linear function of excitation power, b) internal relaxation in the excited-electronic state, c) radiative or non-radiative decay from the excited state to the ground state as determined by the excited state lifetime, and d) internal relaxation in the ground state. Single molecule fluorescence measurements are considered digital in nature because the measurement relies on a signal/no signal readout independent of the intensity of the signal.

The high dynamic-range analyte quantification methods of the invention allow the measurement of over 10,000 analytes from a biological sample. The method can quantify analytes with concentrations from about 1 ag/mL to about 50 mg/mL and produce a dynamic range of more than 1010. The optical signals are digitized, and analytes are identified based on a code (ID code) of digital signals for each analyte.

As described above, in certain embodiments, analytes are bound to a solid substrate, and probes are bound to the analytes. Each of the probes comprises tags and specifically binds to a target analyte. In some embodiments, the tags are fluorescent molecules that emit the same fluorescent color, and the signals for additional fluors are detected at each subsequent pass. During a pass, a set of probes comprising tags are contacted with the substrate allowing them to bind to their targets. An image of the substrate is captured, and the detectable signals are analyzed from the image obtained after each pass.

The information about the presence and/or absence of detectable signals is recorded for each detected position (e.g., target analyte) on the substrate.

In some embodiments, the invention comprises methods that include steps for detecting optical signals emitted from the probes comprising tags, counting the signals emitted during multiple passes and/or multiple cycles at various positions on the substrate, and analyzing the signals as digital information using a K-bit based calculation to identify each target analyte on the substrate. Error correction can be used to account for errors in the optically-detected signals, as described below.

In some embodiments, a substrate is bound with analytes comprising N target analytes. To detect N target analytes, M cycles of probe binding and signal detection are chosen. Each of the M cycles includes 1 or more passes, and each pass includes N sets of probes, such that each set of probes specifically binds to one of the N target analytes. In certain embodiments, there are N sets of probes for the N target analytes.

In each cycle, there is a predetermined order for introducing the sets of probes for each pass. In some embodiments, the predetermined order for the sets of probes is a randomized order. In other embodiments, the predetermined order for the sets of probes is a non-randomized order. In one embodiment, the non-random order can be chosen by a computer processor. The predetermined order is represented in a key for each target analyte. A key is generated that includes the order of the sets of probes, and the order of the probes is digitized in a code to identify each of the target analytes.

In some embodiments, each probe or probe set is associated with a distinct tag for detecting the target analyte, and the number of distinct tags is less than the number of N target analytes. In that case, each N target analyte is matched with a sequence of M tags for the M cycles. The ordered sequence of tags is associated with the target analyte as an identifying code.

(vii) Devices for Single Molecular Detection

Optical detection requires an optical detection instrument or reader to detect the signal from the labeled probes. U.S. Pat. Nos. 8,428,454 and 8,175,452, which are incorporated by reference in their entireties, describe exemplary imaging systems that can be used and methods to improve the systems to achieve sub-pixel alignment tolerances. In some embodiments, methods of aptamer-based microarray technology can be used. See Optimization of Aptamer Microarray Technology for Multiple Protein Targets, Analytica Chimica Acta 564 (2006).

(viii) Quantification of Optically-Detected Probes

After the detection process, the signals from each probe pool are counted, and the presence or absence of a signal and the color of the signal can be recorded for each position on the substrate.

From the detectable signals, K bits of information are obtained in each of M cycles for the N distinct target analytes. The K bits of information are used to determine L total bits of information, such that K×M=L bits of information and L>log 2 (N). The L bits of information are used to determine the identity (and presence) of N distinct target analytes. If only one cycle (M=1) is performed, then K×1=L. However, multiple cycles (M>1) can be performed to generate more total bits of information L per analyte. Each subsequent cycle provides additional optical signal information that is used to identify the target analyte.

In practice, errors in the signals occur, and this confounds the accuracy of the identification of target analytes. For instance, probes may bind the wrong targets (e.g., false positives) or fail to bind the correct targets (e.g., false negatives). Methods are provided, as described below, to account for errors in optical and electrical signal detection.

The probes used to detect the analytes are introduced to the substrate in an ordered manner in each cycle. A key is generated that encodes information about the order of the probes for each target analyte. The signals detected for each analyte can be digitized into bits of information. The order of the signals provides a code for identifying each analyte, which can be encoded in bits of information.

(ix) Error-Correction Methods

In optical detection methods described above, errors can occur in binding and/or detection of signals. In some cases, the error rate can be as high as one in five (e.g., one out of five fluorescent signals is incorrect). This equates to one error in every five-cycle sequence. Actual error rates may not be as high as 20%, but error rates of a few percent are possible. In general, the error rate depends on many factors including the type of analytes in the sample and the type of probes used. In an optical detection method, a probe may not bind to its target or bind to the wrong target.

Additional cycles are generated to account for errors in the detected signals and to obtain additional bits of information, such as parity bits. The additional bits of information are used to correct errors using an error correcting code. In an embodiment, the error correcting code is a Reed-Solomon code, which is a non-binary cyclic code used to detect and correct errors in a system. In other embodiments, various other error correcting codes can be used. Other error correcting codes include, for example, block codes, convolution codes, Monte Carlo codes, Golay codes, Hamming codes, BCH codes, AN codes, Reed-Muller codes, Goppa codes, Hadamard codes, Walsh codes, Hagelbarger codes, polar codes, repetition codes, repeat-accumulate codes, erasure codes, online codes, group codes, expander codes, constant-weight codes, tornado codes, low-density parity check codes, maximum distance codes, burst error codes, luby transform codes, fountain codes, and raptor codes. See Error Control Coding, 2nd Ed., S. Lin and DJ Costello, Prentice Hall, New York, 2004.

Error correction can reduce the false-positive detection rate to less than 1 in 104, less than 1 in 105, less than 1 in 107, less than 1 in 108 or less than 1 in 109.

### Generalized Description of Specific Embodiments for Detection of Nucleotide Sequence Variants, Alleles and Single Nucleotide Polymorphisms of Interest

(x) Embodiments Comprising a Ligation Reaction Product

In an embodiment, the application describes methods for the detection of target nucleotide sequence variants (e.g., alleles, single nucleotide polymorphisms, mutations, low incidence mutation, etc.) comprising providing a ligation reaction product of a target-dependent oligonucleotide ligation reaction performed on an enriched nucleic acid sample. The enriched nucleic acid sample can be or be derived from any nucleic acid found in biological material, such as, but not limited to genomic DNA, mRNA, mitochondrial DNA, cDNA. In an aspect, the enriched nucleic acid sample is enriched by performing a reverse transcription reaction on a sample comprising RNA. In certain embodiments, the ligation reaction product is generated by hybridizing allele-specific oligonucleotides probes or sequence variant-specific oligonucleotide probes and locus-specific oligonucleotide probes to an enriched nucleic acid sample. In certain aspects, the allele-specific oligonucleotides and locus-specific oligonucleotides are aligned for ligation when hybridized to the target nucleotide sequence variants and the allele-specific oligonucleotide probe and locus specific oligonucleotide probes and can be ligated to each other. In certain aspects, the allele-specific oligonucleotides and locus-specific oligonucleotides are adjacent to each other when hybridized to the target nucleotide sequence variants. The ligation reaction may occur using means known in the art, e.g., using T4 ligase. Attachment or conjugation of nearby or adjacent probes can also be carried out by use of adapters or other means to attach nearby allele-specific and locus-specific probes to each other to produce an allele-specific probe and locus-specific probe conjugate. In an aspect, the ligated or attached allele-specific probes and locus-specific probes can then be denatured. In certain aspects, the ligated allele-specific and locus-specific probes or allele-specific probe and locus specific probe conjugates comprise both a substrate binding moiety and a barcode moiety. In an aspect, the allele-specific probes are bound to a barcode moiety. In an aspect, the locus-specific probes are bound to a substrate binding-moiety. The ligated or attached allele-specific probes and locus-specific probes can be then distributed on a substrate. The ligated or attached allele-specific and locus-specific probes are then distributed and bound onto a substrate using methods described above or any methods known in the art to bind nucleic acid molecules to a substrate. In certain aspects, the ligated or attached allele-specific and locus-specific probes are distributed at spatially separate regions on the substrate. In certain aspects, the probes are distributed in an array format. The support and probes are then washed using an appropriate solution or buffer to remove unbound probes (for example, allele-specific probes not bound to a locus-specific probe, and thus, lack a substrate binding moiety). An appropriate solution or buffer can be any solution that does not substantially interfere with the affinity of the conjugated allele-specific and locus-specific probes with the substrate or change the structure of the oligonucleotides. Methods of detecting nucleic acid sequences using a ligase reaction to anneal probes and arrays to detect ligated probes are described in U.S. Pat. Nos. 5,494,810 and 6,852,487 both of which are incorporated herein by reference in their entirety.

A target nucleotide sequence variant identification assay is then performed to detect the sequence variants using a detection moiety conjugated to barcode probes. In an aspect, barcode probes are complementary to the barcode moieties. In certain aspects, the barcode probes are conjugated with a detection moiety or detection label. The detection label can be a fluorescent tag (i.e., a fluorophore) or any other molecular tag. In certain aspects, the barcode probes may correspond to one or more loci. In certain aspects, the barcode probes are unique for each nucleotide sequence variant. In an aspect, the barcode probes corresponding to a single locus are contacted with the substrate sequentially, and the barcode probes are detected after addition to the substrate prior to contacting the substrate with an additional plurality of barcode probes corresponding to a different locus. In certain aspects, the enriched nucleic acid comprising the nucleotide sequence variants is complementary DNA (cDNA). In certain aspects, barcode probes corresponding to cDNAs corresponding to an individual gene or locus is contacted with the substrate. In an aspect, barcode probes corresponding to different cDNAs corresponding to different genes or loci are contacted with the substrate.

In an aspect, the variant identification assay determines the presence or absence of one or more nucleotide sequence variants. In an aspect, the variant identification assay determines the quantity of one or more nucleotide sequence variants. The variant identification assay comprises performing at least M detection cycles to generate a signal detection sequence, wherein M is at least two. In certain embodiments, each detection cycle comprises contacting the substrate bound to the attached allele-specific probe and locus-specific probe conjugates with a plurality of barcode probes that anneal with the barcode moieties on the substrate, washing the substrate using an appropriate solution or buffer to remove unbound barcode probes, detecting the identity and location of the detection label bound to the barcode probe on the substrate; and if the cycle number is less than M, removing the barcode probe from the barcode moiety; and analyzing the signal detection sequence generated by the M cycles at the spatially separate locations on the substrate to determine the presence or absence of the at least one target nucleotide sequence variant of interest. In certain aspects, the detection of the identity and location of the detection label is performed by optical detection using an optical detection instrument or reader to detect the signal from the labeled probes. Any imaging system can also be used to achieve sub-pixel alignment tolerances. In certain aspects, M is greater than 1, 2, 3, 4, 10, 15, 20, 25, 30, 35, 40, 45, or 50. In certain aspects, M is sufficient to detect a barcode moiety bound to the substrate with a false positive detection rate of less than 1 in 10⁶. Analysis of the signal detection sequence can be performed by comparing the signal detection sequence with an anticipated signal detection sequence for the target nucleotide sequence variant of interest, and determining a probability score for the presence or absence of the target nucleotide sequence variant of interest based on the signal detection sequence. In certain aspects, the analysis reduces the error due to misidentification of the target. In an aspect, a misidentification event is due to a false positive or a false negative signal. In certain aspects, the false-positive rate for the detection of at least one target nucleotide sequence variant of interest is less than 1 in 10⁶. In certain aspects, the false-positive detection rate is less than less than 1 in 10⁴, 1 in 10⁵, less than 1 in 10⁷, less than 1 in 10⁸ or less than 1 in 10⁹. In certain aspects, a target nucleotide sequence variant identification assay is carried out for identifying N nucleotide sequence variants comprising providing at least M sets of barcode probes for performing at least M cycles of the assay, each set comprising N unique barcode binding moieties capable of binding preferentially to a corresponding one of the N barcode moieties, each barcode probe set comprising a detection label for generating K bits of information per cycle, performing at least M detection cycles to generate a signal detection sequence at a plurality of locations on the substrate and determining from M detection cycles L total bits of information, wherein K×M=L and L>log 2 (N), and wherein the L bits of information are used to identify one or more of the N nucleotide sequence variants. The method can be used for varying degrees of multiplex capabilities. In certain aspects, N corresponds to a plurality of loci. In certain aspects N corresponds to a plurality of alleles for a plurality of loci. In certain aspects, the nucleotide variant identification assay comprises determining L total bits of information such that L is sufficient to reduce a false positive error rate of detection to less than 1 in 10⁶. In certain aspects, the false-positive detection rate is less than less than 1 in 10⁴, 1 in 10⁵, less than 1 in 10⁷, less than 1 in 10⁸ or less than 1 in 10⁹. In an aspect, L is a function of the misidentification rate for a target at each cycle. In an aspect, the misidentification rate comprises the non-binding rate and the false binding rate of the probe set to the barcode. In certain aspects, L comprises bits of information that are ordered in a predetermined order. In certain aspects, the predetermined order is a random order. In certain aspects, L comprises bits of information comprising a key for decoding an order of the plurality of ordered probe reagent sets. In certain aspects, at least K bits of information comprise information about the absence of a signal for one of the N distinct target analytes.

In certain embodiments, the substrate bound to the biological material comprising the target nucleotide sequence variants can be further interrogated by the single nucleotide extension detection methods described herein. In certain embodiments, further interrogation of the biological material by performing the single nucleotide extension detection methods can further detect rare mis-ligation events leading to less error in the detection overall.

In certain embodiments, the methods for the detection of target nucleotide sequence variants comprising a ligation reaction product of a target-dependent oligonucleotide ligation reaction described herein either with or without further interrogation by performing the single nucleotide extension detection methods, can detect target nucleotide sequence variants (e.g., low-incidence alleles) that are present in the biological material at a percentage below 0.01%, below 0.05%, below 0.1%, below 0.5%, or below 1%.

(xi) Embodiments Comprising Contacting a Substrate Bound to an Enriched Nucleic Acid Sample with Nucleotide Sequence Variant Probes

In an embodiment, the application describes methods for the detection of target nucleotide sequence variants (e.g., alleles, single nucleotide polymorphisms, mutations, low incidence mutation, etc.) comprising contacting a substrate bound to an enriched nucleic acid sample with allele-specific probes or target nucleotide sequence variant binding probes (“variant binding probe”). The enriched nucleic acid sample can be or be derived from any nucleic acid found in biological material, such as, but not limited to genomic DNA, mRNA, mitochondrial DNA, cDNA. In an aspect, the enriched nucleic acid sample is enriched by performing a reverse transcription reaction on a sample comprising RNA. The enriched nucleic acid sample can comprise nucleic acid derived from one or more origins. The enriched nucleic acid sample can comprise nucleic acid corresponding to one or more loci of interest. The enriched nucleic acid sample is bound to the support by any methods described above or known in the art. In an aspect, the variant binding probes are capable of each binding preferentially to a corresponding single one of a nucleotide sequence variant at a particular locus. In certain embodiments, the substrate is also contacted with locus-specific probes. In an aspect, the locus-specific probes are capable of binding preferentially to a single locus, comprising one or more nucleotide sequence variants. In certain aspects, a target identification assay is performed where the substrate is contacted first with locus-specific probes, the substrate is washed and then the substrate is contacted with variant binding probes. Contacting of the enriched nucleic acid sample with probes is performed under hybridization conditions with a stringency optimized for the particular probes and sample being assayed. In an aspect, the locus-specific probes are bound to a detection moiety or detection label. In an aspect, the variant binding probes are bound to a detection moiety or detection label. In an aspect, the label is a fluorophore. In certain aspects, the locus-specific probes and the variant binding probes that bind to the same corresponding locus comprise the same detection label regardless of the presence of a particular sequence variant. In certain aspects, the enriched nucleic acid sample is distributed on a substrate so that the nucleic acid sequence variants are bound to the substrate at spatially separate regions on the substrate. A target nucleotide sequence variant identification assay is then preformed. In certain aspects, the target nucleotide sequence variant identification assay determines a quantity of one or more nucleotide sequence variants. The target nucleotide sequence variant identification assay comprises M number of detection cycles. In an embodiment, the detection cycle comprises contacting the substrate bound to the enriched nucleic acid sample and target nucleotide sequence variant binding probes, washing the surface of the substrate with an appropriate solution or buffer to remove unbound probes, detecting the identity and location of the detectable label on the substrate and if the cycle number is less than M, performing a denaturation reaction to remove bound variant binding probe. In an aspect, the presence or absence of the target nucleotide sequence variant is determined from the sequence of detectable labels at the location on the substrate. In certain aspects, the detection of the identity and/or location of the detection label is performed by optical detection using an optical detection instrument or reader to detect the signal from the labeled probes. Any imaging system can also be used to achieve sub-pixel alignment tolerances.

In certain embodiments, the target oligonucleotide sequence variant identification assay comprises identifying at least one of N nucleotide sequence variants, wherein the assay comprises providing at least M sets of sequence variant probes for performing at least M cycles of the assay, wherein each of the sequence variant probes comprise a detection label for generating K bits of information for the corresponding cycle; wherein for at least 2 of the M cycles, the sequence variant probe set comprises N sequence variant probes each capable of binding preferentially to a corresponding single one of the N nucleotide sequence variants; and performing at least M detection cycles to generate a signal detection sequence at the spatially separate regions of the substrate, wherein M is at least 2. The method can be used for varying degrees of multiplex capabilities. In certain aspects, N corresponds to a plurality of loci. In certain aspects N corresponds to a plurality of alleles for a plurality of loci. In an aspect, L total bits of information are determined from the M detection cycles, wherein the L equals the sum of the K bits of information generated at each of the M detection cycles, wherein L>log 2 (N), and wherein the L bits of information are used to identify one or more of the N oligonucleotide sequence variants. In certain aspects, L is a function of the average non-binding rate and the false binding rate of the variant probe set to the corresponding N oligonucleotide sequence variants. In certain aspects, L is sufficient to reduce a false positive detection error rate from a single binding cycle to less than 1 in 10⁵, less than 1 in 10⁶, less than 1 in 10⁷, less than 1 in 10⁸, or less than 1 in 10⁹. In certain aspects, L is sufficient to reduce a false negative error rate from a single cycle for at least one of the N oligonucleotide sequence variants to less than 0.1%, less than 0.01% or less than 0.001% of the false negative error rate from a single cycle. In an aspect, K varies between two or more cycles. In certain aspects, the oligonucleotide sequence variant probe sets for cycles 1 through X are capable of identifying a locus, but not a sequence variant and X<M. In certain aspects, the oligonucleotide sequence variant probe sets for cycles 1 through X comprise N sequence variant probes each capable of binding preferentially to a corresponding single one of N nucleotide sequence variants, and wherein each probe that binds preferentially to a sequence variant at a particular target locus comprises the same detection marker as other sequence variants at the particular target locus for a particular cycle. In certain other aspects, oligonucleotide sequence variant probe sets for cycles 1 through X comprises a plurality of sequence variant probes that bind preferentially to a target locus, but does not bind preferentially to a sequence variant at the target locus. In certain aspects, X is 1. In certain other aspects, X is more than 1. In certain aspects the variant probes have a cross-reactivity with non-target sequence variant at the same loci of greater than 2%, 5%, 10%, 15%, 20%, or 25%. In certain aspects, at least one of the N oligonucleotide sequence variants does not bind to a corresponding oligonucleotide sequence variant probe for at least 10%, at least 20%, at least 30%, or at least 40% of cycles.

In certain aspects, sequence variant probes and/or locus-specific probes are modified. In certain aspects, the amount of probes or the concentration of each of the sequence variant probes and/or locus-specific probes is optimized to account for the difference in binding affinities and cross-reactivity of the individual probes. In certain aspects, the sequence variant probes and/or locus-specific probes are modified with a peptide nucleic acid (PNA) or locked nucleic acid (LNA) to block binding of a label for optimization of detection methods to account for the different binding activities of probes.

(xii) Embodiments Comprising Performing a Single Base Extension Reaction

In certain embodiments, the application describes methods for the detection of target nucleotide sequence variants (e.g., alleles, single nucleotide polymorphisms, mutations, low incidence mutation, etc.) comprising performing a single base extension reaction on an enriched nucleic acid sample bound to a substrate wherein nucleic acids are distributed on the substrate at distinct spatially separate regions on the substrate. The enriched nucleic acid sample can be or be derived from any nucleic acid found in biological material, such as, but not limited to genomic DNA, mRNA, mitochondrial DNA, cDNA. In an aspect, the enriched nucleic acid sample is enriched by performing a reverse transcription reaction on a sample comprising RNA. The enriched nucleic acid sample can comprise nucleic acid derived from one or more origins. The enriched nucleic acid sample can comprise nucleic acid corresponding to one or more loci of interest. The enriched nucleic acid sample is bound to the support by any methods described above or known in the art. In certain aspects, a target nucleotide sequence variant identification assay is performed, comprising performing at least M detection cycles to generate a signal detection sequence. In certain aspects, the detection cycles comprise contacting the substrate with a set of primers each capable of binding preferentially to an oligonucleotide sequence immediately 5′ to the location of one of at least one target sequence variant, thereby forming a hybridized primer or hybridized oligonucleotide bound to the substrate and contacting the substrate with reagents for performing a single nucleotide extension reaction. In certain aspects, the single nucleotide extension reagents comprise at least one nucleotide comprising a detectable label and a terminator. In certain aspects the terminator is ddNTP. In certain aspects, the nucleotides comprise any of ddATP, ddGTP, ddCTP, and ddTTP. The substrate is then exposed to conditions that promote a single nucleotide extension reaction at the 3′ terminus of the primer, and the substrate surface is then washed to remove unbound nucleotides. Methods of detecting nucleic acid sequences using a single base extension reaction are described in the U.S. Patent publication US20050153320 A1, incorporated herein by reference in its entirety. In certain aspects, detecting the identity and location of the detectable label on the substrate is performed; and if the cycle number is less than M, a denaturation reaction is also performed to remove the primers bound to the oligonucleotides. The presence or absence of the target nucleotide sequence variant is then determined from the sequence of detectable labels for each cycle at a location on the substrate. In certain aspects, the detection of the identity and/or location of the detection label is performed by optical detection using an optical detection instrument or reader to detect the signal from the labeled probes. Any imaging system can also be used to achieve sub-pixel alignment tolerances.

In certain aspects, the nucleotide extension reaction at each cycle comprises addition of only one type of a nucleotide. In certain other aspects, the nucleotide extension reaction at each cycle comprises addition of all types of nucleotides comprising adenosine, guanine, thymine, and cytosine. In certain aspects, the detectable label is fluorescent label. In certain aspects, the detectable label corresponds to a unique nucleotide identity. In certain aspects, the single base extension reaction is performed with a set of reagents comprising 4 distinctly labeled ddNTP, wherein each distinctly labeled ddNTP is bound to a distinct fluorophore.

In an embodiment, the target single nucleotide variant identification assay comprises providing a set of primers for each locus comprising at least one of the N single nucleotide variants, contacting the oligonucleotides hybridized to the primers with a set of nucleotides for generating K bits of information for the corresponding cycle, detecting the identity and location of the detection label on the substrate to generate K bits of information at each of the spatially separate regions for the cycle and determining from the at least M detection cycles L total bits of information, wherein the L equals the sum of the K bits of information generated at each of the M detection cycles, wherein L>log₂ (N), and wherein the L bits of information are used to identify one or more of the N oligonucleotide sequence variants. In an aspect, at least K bits of information comprise information about the absence of a signal for one of the N distinct target analytes. In certain aspects, K varies between two or more cycles. In certain other aspects, K is constant for all cycles, and L=K×M. The method can be used for varying degrees of multiplex capabilities. In certain aspects, N corresponds to a plurality of loci. In certain aspects N corresponds to a plurality of alleles for a plurality of loci. In certain aspects, N is at least 10, at least 20, at least 30, at least 40, at least 50, at least 75, at least 100, at least 200, at least 500, or at least 1,000. In certain aspects, L is sufficient to reduce a false positive detection error rate from a single binding cycle to less than 1 in 10⁵, less than 1 in 10⁶, less than 1 in 10⁷, less than 1 in 10⁸, or less than 1 in 109. In certain aspects, L is sufficient to reduce a false negative error rate of detection of at least one of N oligonucleotide sequence variants to less than 0.1%, less than or less than 0.001%. In certain aspects, the method comprises further comprising contacting the oligonucleotides bound to the substrate with a locus specific probe that binds preferentially to a specific locus comprising any of the single nucleotide variants at the locus. In certain aspects, the methods comprise carrying out on the oligonucleotides bound to the substrate a locus identification assay comprising performing Q number of detection cycles for locus identification, wherein Q is at least two, each cycle comprising contacting the oligonucleotides bound to the substrate with a locus binding probe that binds preferentially to the locus, the locus binding probe comprising a detectable label; washing the surface of the substrate to remove unbound locus binding probes; detecting the identity and location of the detectable label on the substrate; and if the cycle number is less than Q, performing a denaturation reaction to remove bound nucleotide sequence variant binding probes or allele binding probes from the oligonucleotide bound to the substrate; and determining from the sequence of detectable labels at the location on the substrate the presence or absence of the nucleotide sequence variant or allele suspected of being present in the sample. In certain aspects, the plurality of oligonucleotides bound to the substrate comprises the + and − strand at the locus, wherein the target single nucleotide variant identification assay is redundantly performed on both the + and − strand. In certain embodiments, the methods can detect target nucleotide sequence variants (e.g., low-incidence alleles) that are present in the biological material at a percentage below 0.01%, below 0.05%, below 0.1%, below 0.5%, or below 1%.

(xiii) Embodiments Comprising Detection of Variant—Specific Amplification Products

In an embodiment, described herein are methods of identifying at least one target nucleotide sequence variant (e.g., alleles, single nucleotide polymorphisms, mutations, low incidence mutation, etc.) in an enriched nucleic acid sample, comprising detection of an amplification reaction product of a sequence variant-specific amplification reaction wherein the amplification reaction product comprises a plurality of oligonucleotides each comprising a substrate binding moiety and a barcode moiety. The enriched nucleic acid sample can be or be derived from any nucleic acid found in biological material, such as, but not limited to genomic DNA, mRNA, mitochondrial DNA, cDNA. In an aspect, the enriched nucleic acid sample is enriched by performing a reverse transcription reaction on a sample comprising RNA. The enriched nucleic acid sample can comprise nucleic acid derived from one or more origins. The enriched nucleic acid sample can comprise nucleic acid corresponding to one or more loci of interest. The amplification reaction product is distributed on a substrate such that individual oligonucleotides bind to the substrate via the substrate binding moiety at spatially separate regions of the substrate. The enriched nucleic acid sample is bound to the support by any of the methods described above or any methods known in the art. In an aspect, the method comprises carrying out on the substrate a target nucleotide sequence variant identification assay, wherein the sequence variant identification assay comprises performing at least M detection cycles to generate a signal detection sequence, wherein M is at least two, each cycle comprising contacting the amplification reaction product with a barcode probe comprising a detection label wherein the barcode probe binds to the barcode moiety when it is present on the substrate; washing the surface of the substrate to remove unbound barcode probes; detecting the identity and location of the detection label on the substrate; and if the cycle number is less than M, removing the barcode probe from the barcode moiety; and analyzing the signal detection sequence generated by the M cycles at the spatially separate locations on the substrate to determine the presence or absence of the at least one target nucleotide sequence variant of interest. Contacting of the enriched nucleic acid sample with barcode probes is performed under hybridization conditions with a stringency optimized for the particular barcode probes and sample being assayed. In certain aspects, the detection of the identity and/or location of the detection label is performed by optical detection using an optical detection instrument or reader to detect the signal from the labeled probes. Any imaging system can also be used to achieve sub-pixel alignment tolerances.

In an aspect, the step of providing the amplification reaction product comprises carrying out the sequence variant-specific amplification reaction on the sample. Methods of performing a sequence variant-specific amplification reaction for certain embodiments are described in more detail below and are also described in U.S. Pat. No. 5,302,509, incorporated herein in its entirety. In an aspect, the sample is an enriched nucleic acid sample suspected of comprising at least one target nucleotide sequence variant of a plurality of sequence variants at one of a plurality of target loci. In certain embodiments, the method comprises carrying out the sequence variant-specific amplification reaction on the sample. In an embodiment, the sequence variant-specific amplification reaction comprises providing a plurality of oligonucleotide primer sets, each set comprising a pair of oligonucleotide primers for amplifying a locus suspected of comprising the oligonucleotide sequence variant. In certain aspects, a primer pair comprises a first oligonucleotide primer capable of specifically hybridizing to one of a plurality of nucleotide sequence variants at a target locus, wherein the primer is bound to a barcode moiety and a second oligonucleotide primer capable of specifically hybridizing to the target locus at a region upstream or downstream from the sequence variant, wherein the second oligonucleotide primer is bound to a substrate binding moiety. Contacting of the enriched nucleic acid sample with primers is performed under hybridization conditions with a stringency optimized for the particular primers and sample being assayed. In certain aspects, the method comprises contacting the sample with the plurality of oligonucleotide primer sets and amplification reagents to perform the sequence variant-specific amplification reaction, thereby generating the amplification reaction product. In certain aspects, more than one barcode moiety is bound to the primer.

In an aspect, the target nucleotide variant identification assay comprises identifying at least one of N nucleotide sequence variants, providing at least M sets of barcode probes for performing at least M cycles of the assay, each set comprising N unique barcode binding moieties capable of binding preferentially to a corresponding one of the N barcode moieties for generating K bits of information per cycle and performing at least M detection cycles to generate a signal detection sequence at a plurality of the spatially separate regions on the substrate, wherein M is at least one. In an aspect, L total bits of information are determined from at least M detection cycles wherein K×M=L and L>log₂ (N), and wherein the L bits of information are used to identify one or more of the N nucleotide sequence variants. In certain aspects, M is greater than 1, 2, 3, 4, 5, 10, 15, 20, 25, 30, 35, 40, 45, or 50. In certain aspects, M is sufficient to detect a barcode moiety bound to the substrate with a false positive detection rate of less than 1 in 10⁶. Analysis of the signal detection sequence can be performed by comparing the signal detection sequence with an anticipated signal detection sequence for the target nucleotide sequence variant of interest, and determining a probability score for the presence or absence of the target nucleotide sequence variant of interest based on the signal detection sequence. In certain aspects, the analysis reduces the error due to misidentification of the target. In an aspect, a misidentification event is due to a false positive or a false negative signal. In certain aspects, the false-positive rate for the detection of at least one target nucleotide sequence variant of interest is less than 1 in 10⁶. In certain aspects, the false-positive detection rate is less than less than 1 in 10⁴, 1 in 10⁵, less than 1 in 10⁷, less than 1 in 10⁸ or less than 1 in 10⁹. In certain aspects, the nucleotide variant identification assay comprises determining L total bits of information such that L is sufficient to reduce a false positive error rate of detection to less than 1 in 10⁶. In an aspect, L is a function of the misidentification rate for a target at each cycle. In an aspect, the misidentification rate comprises the non-binding rate and the false binding rate of the probe set to the barcode. In certain aspects, L comprises bits of information that are ordered in a predetermined order. In certain aspects, the predetermined order is a random order. In certain aspects, L comprises bits of information comprising a key for decoding an order of the plurality of ordered probe reagent sets. In certain aspects, at least K bits of information comprise information about the absence of a signal for one of the N distinct target analytes. The method can be used for varying degrees of multiplex capabilities. In certain aspects, N corresponds to a plurality of loci. In certain aspects N corresponds to a plurality of alleles for a plurality of loci. In certain embodiments, the methods can detect target nucleotide sequence variants (e.g., low-incidence alleles) that are present in the biological material at a percentage below 0.01%, below below 0.1%, below 0.5%, or below 1%.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Carey and Sundberg Advanced Organic Chemistry 3^{rd} Ed. (Plenum Press) Vols A and B (1992).

### Example 1: Detection of Low Frequence Alleles of Interest by Detection of a Ligation Reaction Product

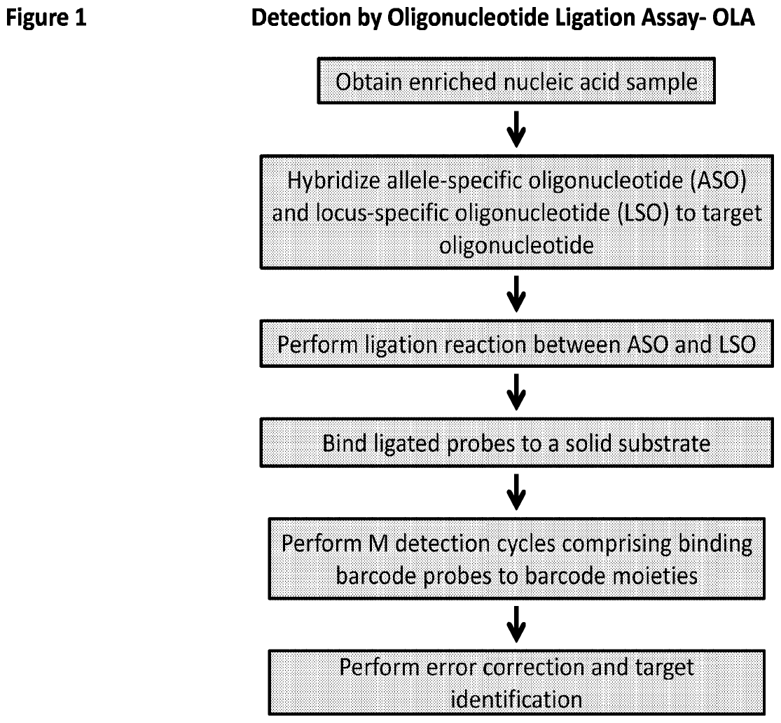

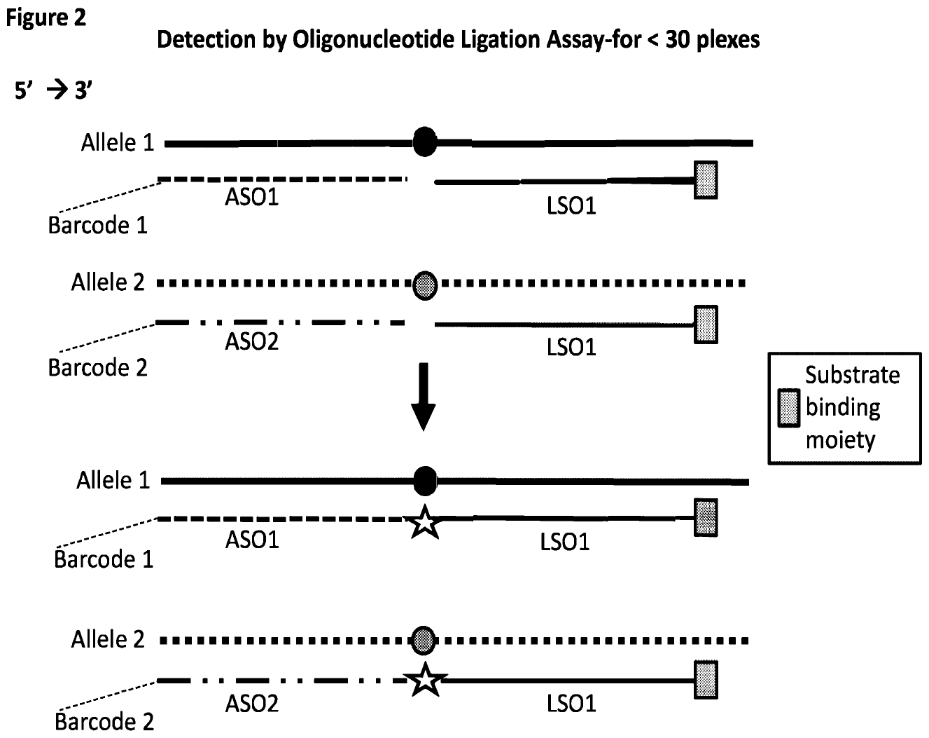

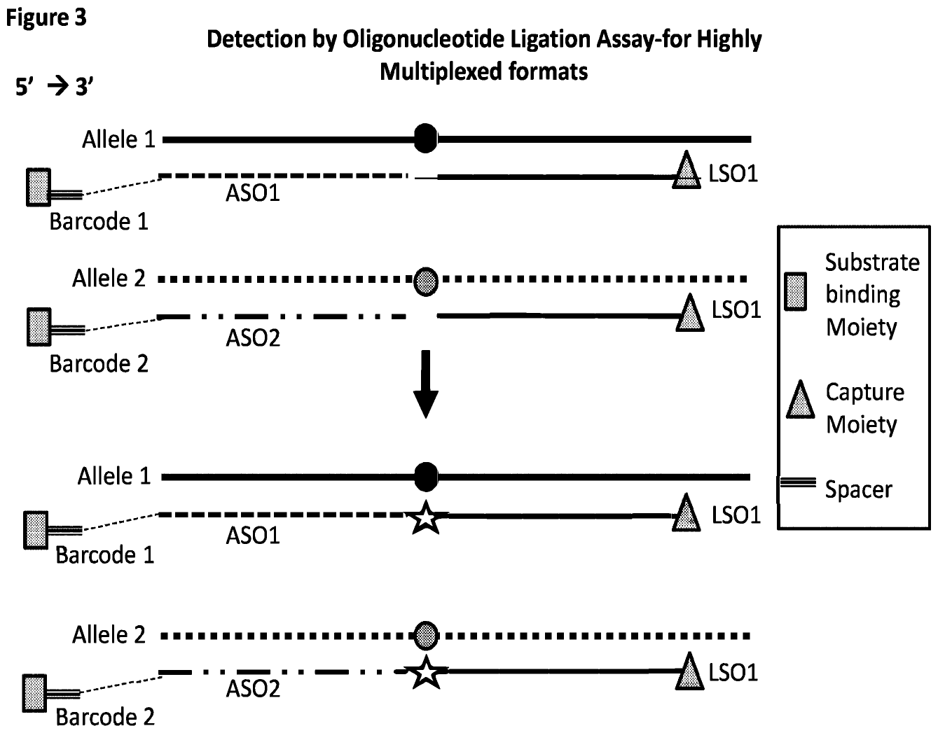

Genomic DNA is extracted from patient samples according to known methods. The genomic DNA is then fragmented by heat-mediated fragmentation by incubating the samples for 2-5 minutes at 99° C. The concentration DNA in each sample is 50-200 ng/uL and the volume of 12.5 to 150 uL in water or 1×TE. Fragmentation is performed to generate lengths of nucleic acids less than 12 kilobases, preferably 2 to 7 kbases. An oligonucleotide ligation assay followed by detection is then performed on the fragmented, enriched nucleic acid sample as outlined in FIG. 1. Examples of locus-specific oligonucleotide (LSO) probes and allele-specific oligonucleotide (ASO) probes for detection of mutations in two genes, BRAF and EGFR, are shown in Table 1 below. Oligonucleotide ligation reactions (OLA) are performed using the SNPlex™ Genotyping System 48-plex system available from Applied Biosystems™. 48 locus-specific oligonucleotide probes and 96 allele-specific oligonucleotide probes are added to the fragmented genomic DNA samples and allowed to hybridize to the fragmented genomic DNA under high or low stringency conditions such as, hybridizing in a solution of 1×SSC at pH7, Sodium dodecyl sulfate (SDS), 1% Bovine Serum Albumin for 18-24 hours at 42° C. In addition, 96 Allele-specific oligonucleotide linkers or adapters comprising barcode moieties and sequences to direct the binding of each linker to a particular allele-specific oligonucleotide probe and a single locus-specific oligonucleotide linker capable of annealing to any of the 48 locus-specific oligonucleotide probes are also added to the fragmented genomic DNA and allowed to hybridize. The locus-specific oligonucleotide probes linkers comprise the substrate binding moiety of biotin. The allele-specific oligonucleotide probes and locus specific probes are ligated to each other, and the linkers are ligated to the corresponding oligonucleotide probes using T4 DNA ligase (New England Biolabs). Alternatively, oligonucleotide ligation reactions are performed using locus-specific oligonucleotide probes and allele-specific probes in the absence of linkers or adapters, and barcode moieties are conjugated to the allele-specific probes (FIG. 2 and FIG. 3).

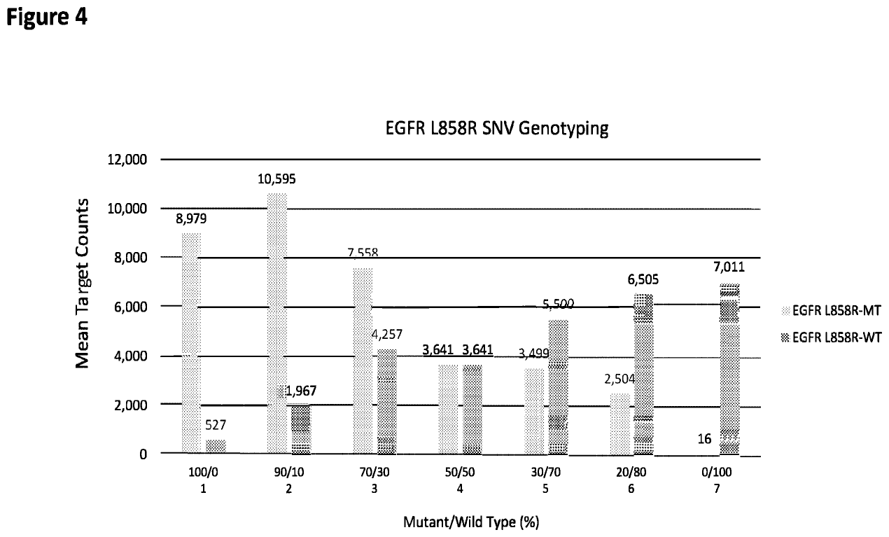

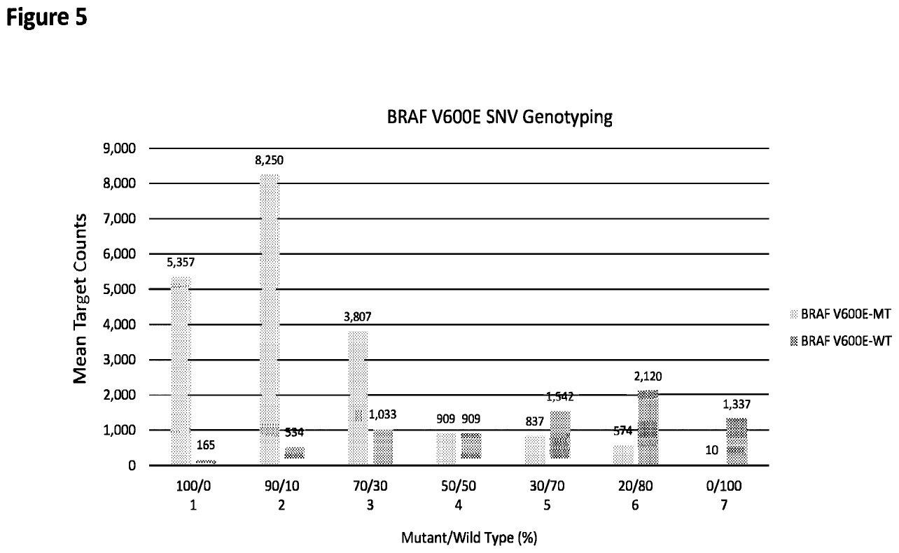

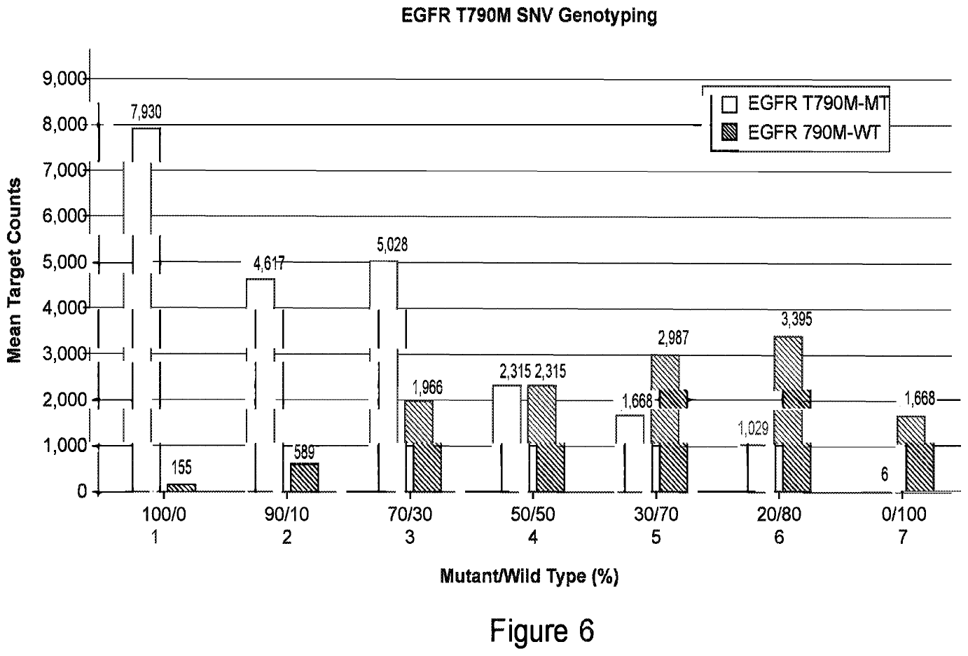

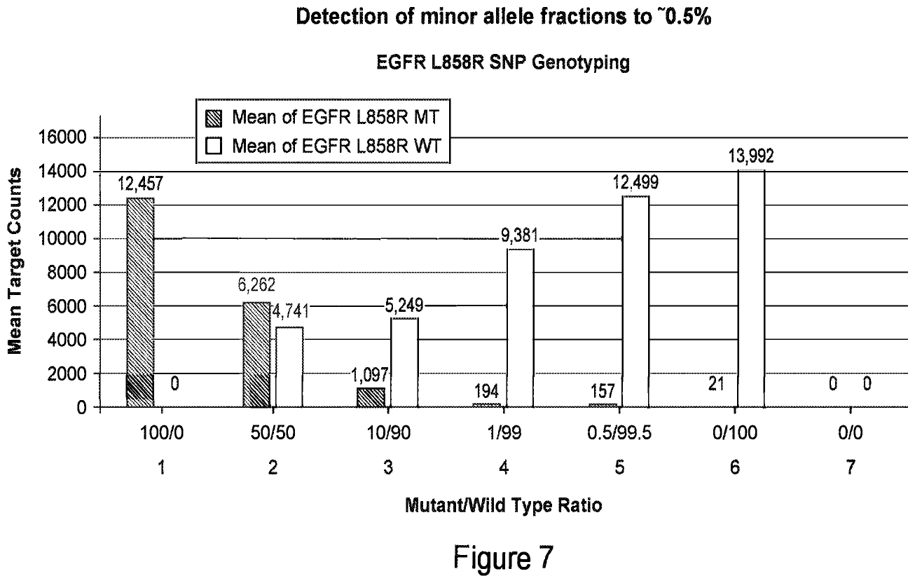

The ligation products are then contacted with exonucleases to digest portions of the ligated OLA reaction products, unligated and partially ligated oligonucleotides and the genomic DNA. The ligation products are then distributed on a streptavidin-coated glass slide wherein the streptavidin is coated in an array format. Fluorescent-tagged barcode probes corresponding to individual allele-specific probes are then added for each locus of interest sequentially to the coated slide. Each of the two allele-specific probes corresponding to each allele of a specific locus are tagged with a unique fluorophore, (such as, GFP, RFP etc.). The alleles are detected by performing M=10 cycles to generate a reduced false-positive error rate, wherein each cycle comprises contacting the slide with the allele-specific probes corresponding to an individual locus, washing the slide to remove unbound barcode probe and detecting the fluorescence at each region on the array using an optical imaging system (GenePix® 4200A microarray scanner provided by Axon Instruments™). If the cycle is less than 10, the cycle further comprises denaturing the barcode probes from the array. In each cycle, the bar code probes are hybridized to the slide. The barcode probes are added to a solution of 1×SSC at pH7, 0.1% Sodium dodecyl sulfate (SDS), 1% Bovine Serum Albumin for 18-24 hours at 42° C. The washing conditions for removing unbound barcode probes are carried out by washing the array with 2×SSC at pH7, 0.1% SDS at 42° C. for 5 minutes then washed either in low stringency conditions (one wash with 0.1×SSC, 0.1% SDS for 10 minutes at room temperature) or high stringency conditions (washed four times 0.1×SSC, 0.1% SDS for 5 minutes at 60° C.). After the step of denaturing the barcode probes to remove bound barcode probes following the detection step and washing the barcode probes from the array, the array is scanned to confirm efficient removal or stripping of the barcode probes prior to initiating the subsequent cycle. Analysis of color codes for identification of sequences is performed using a two-color imaging system. Mapping of target identification sequence to color sequence is performed such that each color corresponds to a sequence, which maps to 1 or 0 with 1 bit of information being acquired per cycle. The error correction scheme is conservative and requires zero errors per target, an error is defined as a positive identification in a sequence where it is not expected. Up to five missing sequences are allowed per molecule. Missing sequences are cases where a molecule is not identified in a cycle and are not classified as errors. In certain examples, the array is further interrogated using the detection methods comprising a single nucleotide extension reaction as described herein. Single nucleotide variants of Epidermal Growth Factor Receptor and BRAF were detected by performing oligonucleotide ligation reactions (OLA) as described above in a multiplexed format. Genotyping results for detection of the EGFR allele harboring the mutation L858R are shown in FIG. 4. Genotyping results for detection of the BRAF allele harboring the V600E mutation are shown in FIG. 5. Genotyping results for detection of the EGFR allele harboring the mutation T790M are shown in FIG. 6. Genoyping results for the detection of the EGFR allele harboring the L858R mutation, where the mutation is present at an allele frequency of 0.5%, are shown in FIG. 7. These results confirm the detection of single nucleotide mutations in low frequency alleles by the oligonucleotide ligation assay (OLA) methods described herein.

TABLE 1
Probes for Detection Using Oligonucleotide Ligation

| | | | | | AS01 | AS02 | | |
|---|---|---|---|---|---|---|---|---|
| | | | | LSO Probe | Probe | Probe | Wild | |
| Gene | COSMIC ID | CDS Mutation | AA Mutation | Sequence | Sequence | Sequence | Type | Mutation |
| BRAF | C0SM476 | c.17991 > A | p. V600E | SEQ ID | SEQ ID | SEQ ID | T | A |
| | | (Substitution, | (Substitution-Missense, | NO: 1 | NO: 2 | NO: 3 | | |
| | | position 1799, | position 600, V→E) | | | | | |
| | | T→A) | | | | | | |
| EGFR | COSM6224 | c.2573T > G | p.L858R | SEQ ID | SEQ ID | SEQ ID | T | G |
| | | (Substitution, | (Substitution-Missense, | NO: 4 | NO: 5 | NO: 6 | | |
| | | position 2573, | position 858, L→R) | | | | | |
| | | T→G) | | | | | | |
| EGFR | COSM6240 | c.2369C > T | p.T790M | SEQ ID | SEQ ID | SEQ ID | C | T |
| | | (Substitution, | (Substitution-Missense, | NO: 7 | NO: 8 | NO: 9 | | |
| | | position 2369, | position 790, T→M) | | | | | |
| | | C→T) | | | | | | |
| BRAF | COSM476 | c.1799T > A | p. V600E | SEQ ID | SEQ ID | SEQ ID | T | A |
| | | (Substitution, | (Substitution-Missense, | NO: 10 | NO: 11 | NO: 12 | | |
| | | position 1799, | position 600, V→E) | | | | | |
| | | T→A) | | | | | | |
| EGFR | C0SM6224 | c.25731 > G | p.L858R | SEQ ID | SEQ ID | SEQ ID | T | G |
| | | (Substitution, | (Substitution-Missense, | NO: 13 | NO: 14 | NO: 15 | | |
| | | position 2573, | position 858, L→R) | | | | | |
| | | T→G) | | | | | | |
| EGFR | C0SM6240 | c.2369C > T | p.T790M | SEQ ID | SEQ ID | SEQ ID | C | T |
| | | (Substitution, | (Substitution-Missense, | NO: 16 | NO: 17 | NO: 18 | | |
| | | position 2369, | position 790, T→M) | | | | | |
| | | C→T) | | | | | | |

### Example 2: Detection of Alleles by Contacting a Substrate Bound to an Enriched Nucleic Acid Sample with Allele-Specific Probes

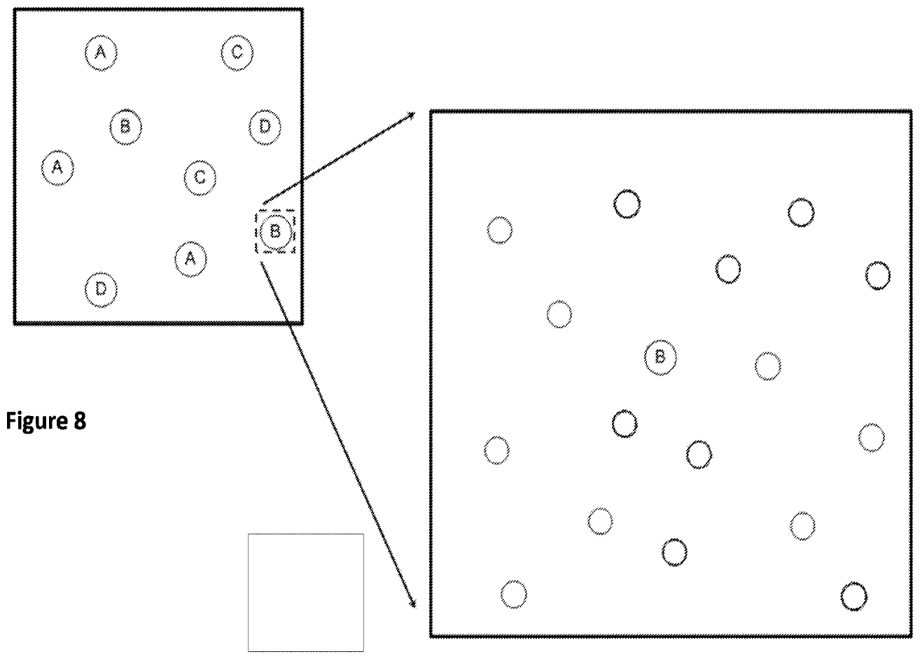

Fragmented genomic DNA prepared as described above in Example 1 are bound and randomly distributed onto the surface of coated silicone slide in an array format (FIG. 8). Silicon slides are purchased from University Wafer (Boston, MA), diced (American Precision Dicing Inc., San Jose, California), and coated with SuperEpoxy substrate (ArrayIt™). The single crystal silicon chips as prepared as 25 mm×75 mm substrate slides. The thickness of the silicon chips used are 500 μm, 675 μm, and 1000 μm. A thermal oxide is grown on the silicon chips of 100 nm and then are diced into slides. The genomic DNA fragments are modified with C6-amino linkers to generate an active primary amino group on the 5′terminus of the genomic DNA fragments (amino linker C6 can be purchased from Gene Link™). The fragmented genomic DNA is denatured into single stranded DNA by incubating the genomic DNA at greater than 80° C. for 10 minutes. The C6 modified single-stranded DNAs are then added to the epoxy coated silicon slides in a container at room temperature overnight. During incubation, a reaction between the epoxy coating and the C6 oligonucleotides covalently bonded the single stranded DNA to the surface.

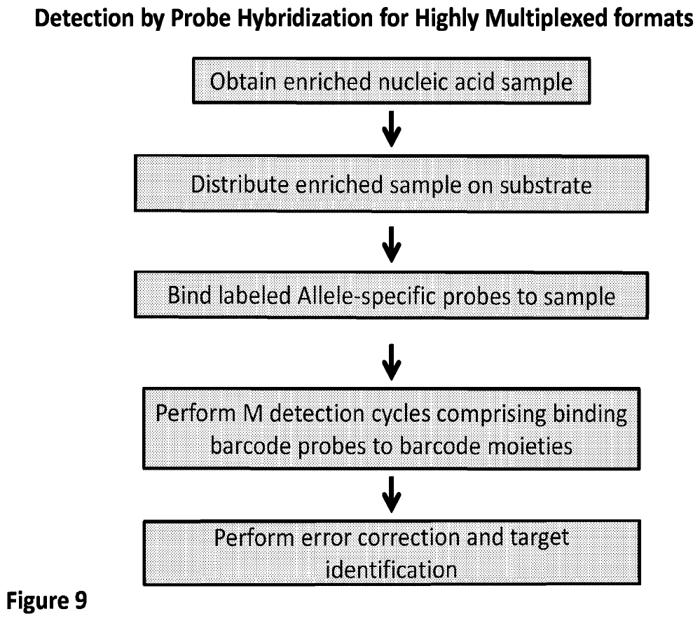

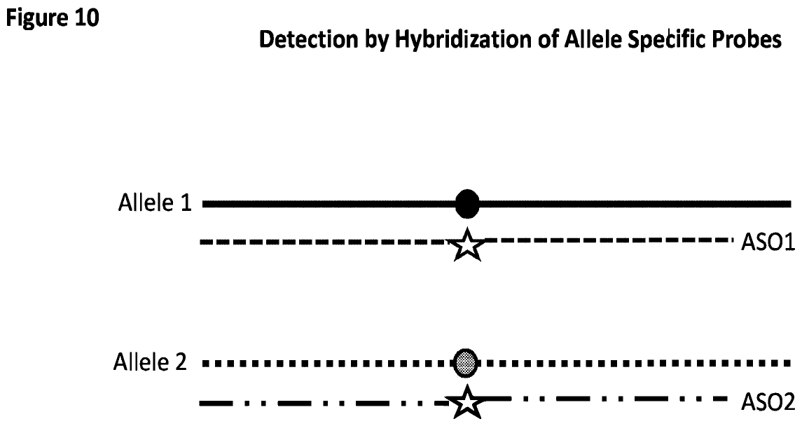

Hybridization of allele-specific probes followed by detection is then performed on the fragmented, enriched nucleic acid sample as outlined in FIG. 9. Allele-specific oligonucleotide probes comprising fluorescent tags are hybridized to the genomic DNA fragments bound on the array under high or low stringency conditions (FIG. 10). Examples of allele-specific oligonucleotide probes specific for wild-type or mutant alleles of EGFR and KRAS genes are shown in Table 2 below. The fluorescent-tagged allele-specific probes are added for each locus of interest sequentially to the coated slide. Each of the allele-specific probes corresponding to each allele of a specific locus are tagged with a unique fluorophore, (such as, GFP, YFP, RFP, etc.). The alleles are detected by performing M=10 cycles to generate a reduced false-positive error rate, wherein each cycle comprises contacting the slide with the allele-specific probes corresponding to an individual locus, washing the slide to remove unbound barcode probe and detecting the fluorescence at each region on the array using an optical imaging system (GenePix® 4200A microarray scanner provided by Axon Instruments™). If the cycle is less than 10, the cycle further comprises denaturing the allele-specific probes from the array. Analysis of color codes for identification of sequences is performed using a two-color imaging system. Mapping of target identification sequence to color sequence is performed such that each color corresponds to a sequence, which maps to 1 or 0 with 1 bit of information being acquired per cycle. The error correction scheme is conservative and requires zero errors per target, an error is defined as a positive identification in a sequence where it is not expected. Up to five missing sequences are allowed per molecule. Missing sequences are cases where a molecule is not identified in a cycle and are not classified as errors.

TABLE 2
Probes for Detection by Hybridization of Allele-Specific Probes
Table 2: Probes for Detection by Hybridization of Allele-Specific Probes

| | COSMIC | CDS | AA | | Probe Sequence- | Probe ID- | Probe Sequence- | Wild | Muta- |
|---|---|---|---|---|---|---|---|---|---|
| Gene | ID | Mutation | Mutation | Probe ID-Wild Type | Wild Type | Mutation | Mutation | Type | tion |
| EGFR | COSM13 | c.2572_ | p.L858R | EGFR_p.858_c53_wt | SEQ ID NO: 19 | EGFR_p.858_ | SEQ ID NO: 20 | CT | AG |
| | 553 | 2573CT > | | | | c53 mut4 | | | |
| | | A G | | | | | | | |
| EGFR | COSM13 | c.2572_ | p.L858R | EGFR_p.858_c54_wt | SEQ ID NO: 21 | EGFR_p.858_ | SEQ ID NO: 22 | CT | AG |
| | 553 | 2573CT > | | | | c54 mut4 | | | |
| | | A G | | | | | | | |
| EGFR | COSM62 | c.2369C > | p.T790M | EGFR_p.790_c44_wt | SEQ ID NO: 23 | EGFR_p.790_ | SEQ ID NO: 24 | C | T |
| | 40 | T | | | | c44 mut1 | | | |
| EGFR | COSM62 | c.2369C > | p.T790M | EGFR_p.790_c50_wt | SEQ ID NO: 25 | EGFR_p.790_ | SEQ ID NO: 26 | C | T |
| | 40 | T | | | | c50 mut1 | | | |
| EGFR | COSM62 | c.2369C > | p.T790M | EGFR_p.790_c57_wt | SEQ ID NO: 27 | EGFR_p.790_ | SEQ ID NO: 28 | C | T |
| | 40 | T | | | | c57 mut1 | | | |
| EGFR | COSM62 | c.2369C > | p.T790M | EGFR_p.790_c59_wt | SEQ ID NO: 29 | EGFR p.790_ | SEQ ID NO: 30 | C | T |
| | 40 | T | | | | c59 mut1 | | | |
| EGFR | COSM62 | c.2369C > | p.T790M | EGFR_p.790_c62_wt | SEQ ID NO: 31 | EGFR p.790_ | SEQ ID NO: 32 | C | T |
| | 40 | T | | | | c62 mut1 | | | |
| EGFR | COSM62 | c.2369C > | p.T790M | EGFR_p.790_c63_wt | SEQ ID NO: 33 | EGFR_p.790_ | SEQ ID NO: 34 | C | T |
| | 40 | T | | | | c63 mut1 | | | |
| EGFR | COSM62 | c.2369C > | p.T790M | EGFR_p.790_c65_wt | SEQ ID NO: 35 | EGFR_p.790_ | SEQ ID NO: 36 | C | T |
| | 40 | T | | | | c65 mut1 | | | |
| EGFR | COSM62 | c.2369C > | p.T790M | EGFR_p.790_c66_wt | SEQ ID NO: 37 | EGFR_p.790_ | SEQ ID NO: 38 | C | T |
| | 40 | T | | | | c66 mut1 | | | |
| EGFR | COSM62 | c.2369C > | p.T790M | EGFR_p.790_c67_wt | SEQ ID NO: 39 | EGFR_p.790_ | SEQ ID NO: 40 | C | T |
| | 40 | T | | | | c67 mut1 | | | |
| EGFR | COSM62 | c.2369C > | p.T790M | EGFR_p.790_c68_wt | SEQ ID NO: 41 | EGFR_p.790_ | SEQ ID NO: 42 | C | T |
| | 40 | T | | | | c68 mut1 | | | |
| EGFR | COSM62 | c.2369C > | p.T790M | EGFR_p.790_c69_wt | SEQ ID NO: 43 | EGFR_p.790_ | SEQ ID NO: 44 | C | T |
| | 40 | T | | | | c69 mut1 | | | |
| EGFR | COSM62 | c.2369C > | p.T790M | EGFR_p.790_c70_wt | SEQ ID NO: 45 | EGFR_p.790_ | SEQ ID NO: 46 | C | T |
| | 40 | T | | | | c70 mut1 | | | |
| EGFR | COSM62 | c.2369C > | p.T790M | EGFR_p.790_c72_wt | SEQ ID NO: 47 | EGFR_p.790_ | SEQ ID NO: 48 | C | T |
| | 40 | T | | | | c72_mut1 | | | |
| EGFR | COSM62 | c.2369C > | p.T790M | EGFR_p.790_c73_wt | SEQ ID NO: 49 | EGFR_p.790_ | SEQ ID NO: 50 | C | T |
| | 40 | T | | | | c73_mut1 | | | |
| EGFR | COSM62 | c.2369C > | p.T790M | EGFR_p.790_c74_wt | SEQ ID NO: 51 | EGFR_p.790_ | SEQ ID NO: 52 | C | T |
| | 40 | T | | | | c74_mut1 | | | |
| EGFR | COSM62 | c.2369C > | p.T790M | EGFR_p.790_c75_wt | SEQ ID NO: 53 | EGFR_p.790_ | SEQ ID NO: 54 | C | T |
| | 40 | T | | | | c75_mut1 | | | |
| EGFR | COSM62 | c.2369C > | p.T790M | EGFR_p.790_c76_wt | SEQ ID NO: 55 | EGFR_p.790_ | SEQ ID NO: 56 | C | T |
| | 40 | T | | | | c76_mut1 | | | |
| EGFR | COSM62 | c.2369C > | p.T790M | EGFR_p.790_c77_wt | SEQ ID NO: 57 | EGFR_p.790_ | SEQ ID NO: 58 | C | T |
| | 40 | T | | | | c77_mut1 | | | |
| EGFR | COSM62 | c.2369C > | p.T790M | EGFR_p.790_c78_wt | SEQ ID NO: 59 | EGFR_p.790_ | SEQ ID NO: 60 | C | T |
| | 40 | T | | | | c78_mut1 | | | |
| EGFR | COSM62 | c.2369C > | p.T790M | EGFR_p.790_c79_wt | SEQ ID NO: 61 | EGFR_p.790_ | SEQ ID NO: 62 | C | T |
| | 40 | T | | | | c79_mut1 | | | |
| EGFR | COSM62 | c.2369C > | p.T790M | EGFR_p.790_c80_wt | SEQ ID NO: 63 | EGFR_p.790_ | SEQ ID NO: 64 | C | T |
| | 40 | T | | | | c80_mut1 | | | |
| EGFR | COSM62 | c.2369C > | p.T790M | EGFR_p.790_c81_wt | SEQ ID NO: 65 | EGFR_p.790_ | SEQ ID NO: 66 | C | T |
| | 40 | T | | | | c81_mut1 | | | |
| EGFR | COSM62 | c.2369C > | p.T790M | EGFR_p.790_c82_wt | SEQ ID NO: 67 | EGFR_p.790_ | SEQ ID NO: 68 | C | T |
| | 40 | T | | | | c82_mut1 | | | |
| EGFR | COSM62 | c.2369C > | p.T790M | EGFR_p.790_c83_wt | SEQ ID NO: 69 | EGFR p.790_ | SEQ ID NO: 70 | C | T |
| | 40 | T | | | | c83_mut1 | | | |
| EGFR | COSM62 | c.2369C > | p.T790M | EGFR_p.790_c85_wt | SEQ ID NO: 71 | EGFR_p.790_ | SEQ ID NO: 72 | C | T |
| | 40 | T | | | | c85_mut1 | | | |
| EGFR | COSM62 | c.2369C > | p.T790M | EGFR_p.790_c86_wt | SEQ ID NO: 73 | EGFR_p.790_ | SEQ ID NO: 74 | C | T |
| | 40 | T | | | | c86_mut1 | | | |
| EGFR | COSM62 | c.2369C > | p.T790M | EGFR_p.790_c87_wt | SEQ ID NO: 75 | EGFR_p.790_ | SEQ ID NO: 76 | C | T |
| | 40 | T | | | | c87_mut1 | | | |
| EGFR | COSM62 | c.2369C > | p.T790M | EGFR_p.790_c88_wt | CSEQ ID NO: 77 | EGFR_p.790_ | SEQ ID NO: 78 | C | T |
| | 40 | T | | | | c88_mut1 | | | |
| EGFR | COSM62 | c.2369C > | p.T790M | EGFR_p.790_c89_wt | SEQ ID NO: 79 | EGFR_p.790_ | SEQ ID NO: 80 | C | T |
| | 40 | T | | | | c89_mut1 | | | |
| EGFR | COSM62 | c.2369C > | p.T790M | EGFR_p.790_c90_wt | SEQ ID NO: 81 | EGFR_p.790_ | SEQ ID NO: 82 | C | T |
| | 40 | T | | | | c90_mut1 | | | |
| EGFR | COSM13 | c.2573_ | p.L858 R | EGFR_p.858_c48_wt | SEQ ID NO: 83 | EGFR_p.858_ | SEQ ID NO: 84 | TG | GA |
| | 3630 | 2574TG > | | | | c48 mut2 | | | |
| | | G A | | | | | | | |
| EGFR | COSM13 | c.2573_ | p.L858 R | EGFR_p.858_c53_wt | SEQ ID NO: 85 | EGFR_p.858_ | SEQ ID NO: 86 | TG | GA |
| | 3630 | 2574TG > G | | | | c53 mut2 | | | |
| | | A | | | | | | | |
| EGFR | COS M13 | c.2573_ | p.L858 R | EGFR_p.858_c54_wt | SEQ ID NO: 87 | EGFR_p.858_ | SEQ ID NO: 88 | TG | GA |
| | 3630 | 2574TG > | | | | c54 mut2 | | | |
| | | G A | | | | | | | |
| EGFR | COS M12 | c.2573 | p.L858 R | EGFR_p.858_c48_wt | SEQ ID NO: 89 | EGFR_p.858_ | SEQ ID NO: 90 | TG | GT |
| | 429 | 2574TG > | | | | c48 mut1 | | | |
| | | G T | | | | | | | |
| EGFR | COSM62 | c.2573T > | p.L858R | EGFR_p.858_c33_wt | SEQ ID NO: 91 | EGFR_p.858_ | SEQ ID NO: 92 | T | G |
| | 24 | G | | | | c33 mut3 | | | |
| KRAS | COSM52 | c.35G > A | p.G12D | KRAS_p.12_c82_wt | SEQ ID NO: 93 | KRAS_p.12_ | SEQ ID NO: 94 | G | A |
| | 1 | | | | | c82 mut5 | | | |
| KRAS | COSM52 | c.35G > A | p.G12D | KRAS_p.12_c85_wt | SEQ ID NO: 95 | KRAS_p.12 | SEQ ID NO: 96 | G | A |
| | 1 | | | | | c85 mut5 | | | |
| KRAS | COSM52 | c.35G > A | p.G12D | KRAS_p.12_c89_wt | SEQ ID NO: 97 | KRAS_p.12_ | SEQ ID NO: 98 | G | A |
| | 1 | | | | | c89 mut5 | | | |
| KRAS | COSM52 | c.35G > A | p.G12D | KRAS_p.12_c90_wt | SEQ ID NO: 99 | KRAS_p.12_ | SEQ ID NO: 100 | G | A |
| | 1 | | | | | c90_mut5 | | | |
| KRAS | COSM52 | c.35G > C | p.G12A | KRAS_p.12_c82_wt | SEQ ID NO: 101 | KRAS_p.12_ | SEQ ID NO: 102 | G | C |
| | 2 | | | | | c82_mut4 | | | |
| KRAS | COSM52 | c.35G > C | p.G12A | KRAS_p.12_c85_wt | SEQ ID NO: 103 | KRAS_p.12_ | SEQ ID NO: 104 | G | C |
| | 2 | | | | | c85_mut4 | | | |
| KRAS | COSM52 | c.35G > C | p.G12A | KRAS_p.12_c89_wt | SEQ ID NO: 105 | KRAS_p.12_ | SEQ ID NO: 106 | G | C |
| | 2 | | | | | c89_mut4 | | | |
| KRAS | COSM52 | c.35G > C | p.G12A | KRAS_p.12_c90_wt | SEQ ID NO: 107 | KRAS_p.12_ | SEQ ID NO: 108 | G | C |
| | 2 | | | | | c90_mut4 | | | |
| KRAS | COSM51 | c.34_ | p.G12C | KRAS_p.12_c82_wt | SEQ ID NO: 109 | KRAS_p.12_ | SEQ ID NO: 110 | GGT | TGC |
| | 3 | 36GGT > TGC | | | | c82_mut2 | | | |
| KRAS | COSM51 | c.34 | p.G12C | KRAS_p.12_c85_wt | SEQ ID NO: 111 | KRAS_p.12_ | SEQ ID NO: 112 | GGT | TGC |
| | 3 | 36GGT > TGC | | | | c85_mut2 | | | |
| KRAS | COSM51 | c.34 | p.G12C | KRAS_p.12_c89_wt | SEQ ID NO: 113 | KRAS_p.12_ | SEQ ID NO: 114 | GGT | TGC |
| | 3 | 36GGT > TGC | | | | c89_mut2 | | | |
| KRAS | COSM51 | c.34 | p.G12C | KRAS_p.12_c90_wt | SEQ ID NO: 115 | KRAS_p.12_ | SEQ ID NO: 116 | GGT | TGC |
| | 3 | 36GGT > TGC | | | | c90_mut2 | | | |
| KRAS | COSM14 | c.35 | p.G12D | KRAS_p.12_c82_wt | SEQ ID NO: 117 | KRAS_p.12_ | SEQ ID NO: 118 | GT | AC |
| | 209 | 36GT > AC | | | | c82_mut3 | | | |
| KRAS | COSM14 | c.35_ | p.G12D | KRAS_p.12_c85_wt | SEQ ID NO: 119 | KRAS_p.12_ | SEQ ID NO: 120 | GT | AC |
| | 209 | 36GT > AC | | | | c85_mut3 | | | |
| KRAS | COSM14 | c.35_ | p.G12D | KRAS_p.12_c89_wt | SEQ ID NO: 121 | KRAS_p.12_ | SEQ ID NO: 122 | GT | AC |
| | 209 | 36GT > AC | | | | c89_mut3 | | | |
| KRAS | COSM14 | c.35 | p.G12D | KRAS_p.12_c90_wt | SEQ ID NO: 123 | KRAS_p.12_ | SEQ ID NO: 124 | GT | AC |
| | 209 | 36GT > AC | | | | c90_mut3 | | | |
| KRAS | COSM516 | c.34G > T | p.G12C | KRAS_p.12_c82_wt | SEQ ID NO: 125 | KRAS_p.12_ | SEQ ID NO: 126 | G | T |
| | | | | | | c82_mut1 | | | |
| KRAS | COSM516 | c.34G > T | p.G12C | KRAS_p.12_c85_wt | SEQ ID NO: 127 | KRAS_p.12_ | SEQ ID NO: 128 | G | T |
| | | | | | | c85_mut1 | | | |
| KRAS | COSM516 | c.34G > T | p.G12C | KRAS_p.12_c89_wt | SEQ ID NO: 129 | KRAS_p.12_ | SEQ ID NO: 130 | G | T |
| | | | | | | c89_mut1 | | | |
| KRAS | COSM516 | c.34G > T | p.G12C | KRAS_p.12_c90_wt | SEQ ID NO: 131 | KRAS_p.12_ | SEQ ID NO: 132 | G | T |
| | | | | | | c90_mut1 | | | |
| EG | COSM13 | c.2572_ | p.L858R | EGFR_p.858_c187_wt | SEQ ID NO: 133 | EGFR p.858_ | SEQ ID NO: 134 | CT | AG |
| FR | 553 | 2573CT > | | | | c187_mut4 | | | |
| | | A G | | | | | | | |
| EGFR | COSM13 | c.2572_ | p.L858R | EGFR_p.858_c198_wt | SEQ ID NO: 135 | EGFR_p.858_ | SEQ ID NO: 136 | CT | AG |
| | 553 | 2573CT > | | | | c198 mut4 | | | |
| | | A G | | | | | | | |
| EGFR | COSM13 | c.2572_ | p.L858R | EGFR_p.858_c209_wt | SEQ ID NO: 137 | EGFR p.858_ | SEQ ID NO: 138 | CT | AG |
| | 553 | 2573CT > | | | | c209 mut4 | | | |
| | | A G | | | | | | | |
| EGFR | COSM13 | c.2572_ | p.L858R | EGFR_p.858_c220_wt | SEQ ID NO: 139 | EGFR p.858_ | SEQ ID NO: 140 | CT | AG |
| | 553 | 2573CT > | | | | c220 mut4 | | | |
| | | A G | | | | | | | |
| EGFR | COSM13 | c.2572_ | p.L858R | EGFR_p.858_c264_wt | SEQ ID NO: 141 | EGFR_p.858_ | SEQ ID NO: 142 | CT | AG |
| | 553 | 2573CT > | | | | c264_mut4 | | | |
| | | A G | | | | | | | |
| EGFR | COSM62 | c.2369C > | p.T790M | EGFR_p.790_c194_wt | SEQ ID NO: 143 | EGFR_p.790_ | SEQ ID NO: 144 | C | T |
| | 40 | T | | | | c194 mut1 | | | |
| EGFR | COSM62 | c.2369C > | p.T790M | EGFR_p.790_c198_wt | SEQ ID NO: 145 | EGFR_p.790_ | SEQ ID NO: 146 | C | T |
| | 40 | T | | | | c198_mut1 | | | |
| EGFR | COSM62 | c.2369C > | p.T790M | EGFR_p.790_c204_wt | SEQ ID NO: 147 | EGFR_p.790_ | SEQ ID NO: 148 | C | T |
| | 40 | T | | | | c204 mut1 | | | |
| EGFR | COSM62 | c.2369C > | p.T790M | EGFR_p.790_c215_wt | SEQ ID NO: 149 | EGFR_p.790_ | SEQ ID NO: 150 | C | T |
| | 40 | T | | | | c215 mut1 | | | |
| EGFR | COSM62 | c.2369C > | p.T790M | EGFR_p.790_c226_wt | SEQ ID NO: 151 | EGFR_p.790_ | SEQ ID NO: 152 | C | T |
| | 40 | T | | | | c226 mut1 | | | |
| EGFR | COSM13 | c.2573_ | p.L858R | EGFR_p.858_c187_wt | SEQ ID NO: 153 | EGFR_p.858_ | SEQ ID NO: 154 | TG | GA |
| | 3630 | 2574TG > | | | | c187 mut2 | | | |
| | | G A | | | | | | | |
| EGFR | COSM13 | c.2573_ | p.L858R | EGFR_p.858_c198_wt | SEQ ID NO: 155 | EGFR_p.858_ | SEQ ID NO: 156 | TG | GA |
| | 3630 | 2574TG > | | | | c198 mut2 | | | |
| | | G A | | | | | | | |
| EGFR | COSM133 | c.2573_ | p.L858R | EGFR_p.858_c209_wt | SEQ ID NO: 157 | EGFR_p.858_ | SEQ ID NO: 158 | TG | G |
| | 630 | 2574TG > | | | | c209 mut2 | | | A |
| | | G A | | | | | | | |
| EGFR | COSM13 | c.2573_ | p.L858R | EGFR_p.858_c220_wt | SEQ ID NO: 159 | EGFR_p.858_ | SEQ ID NO: 160 | TG | G |
| | 3630 | 2574TG > | | | | c220 mut2 | | | A |
| | | G A | | | | | | | |
| EGFR | COSM13 | c.2573_ | p.L858R | EGFR_p.858_c264_wt | SEQ ID NO: 161 | EGFR_p.858_ | SEQ ID NO: 162 | TG | G |
| | 3630 | 2574TG > | | | | c264_mut2 | | | A |
| | | G A | | | | | | | |
| EGFR | COSM124 | c.2573_ | p.L858R | EGFR_p.858_c187_wt | SEQ ID NO: 163 | EGFR_p.858_ | SEQ ID NO: 164 | TG | G |
| | 29 | 2574TG > | | | | c187 mut1 | | | T |
| | | G T | | | | | | | |
| EGFR | COSM12 | c.2573_ | p.L858R | EGFR_p.858_c198_wt | SEQ ID NO: 165 | EGFR_p.858_ | SEQ ID NO: 166 | TG | G |
| | 429 | 2574TG > | | | | c198 mut1 | | | T |
| | | G T | | | | | | | |
| EGFR | COSM12 | c.2573_ | p.L858R | EGFR_p.858_c209_wt | SEQ ID NO: 167 | EGFR_p.858_ | SEQ ID NO: 168 | TG | G |
| | 429 | 2574TG > G | | | | c209_mut1 | | | T |
| EGFR | COSM62 | c.2573T > | p.L858R | EGFR_p.858_c187_wt | SEQ ID NO: 169 | EGFR_p.858_ | SEQ ID NO: 170 | T | G |
| | 24 | G | | | | c187 mut3 | | | |
| EGFR | COSM62 | C.2573T > | p.L858R | EGFR_p.858_c198_wt | SEQ ID NO: 171 | EGFR_p.858_ | SEQ ID NO: 172 | T | G |
| | 24 | G | | | | c198 mut3 | | | |
| KRAS | COSM52 | c.35G > A | p.G12D | KRAS_p.12_c187_wt | SEQ ID NO: 173 | KRAS_p.12_ | SEQ ID NO: 174 | G | A |
| | 1 | | | | | c 187 mut5 | | | |
| KRAS | COSM52 | c.35G > A | p.G12D | KRAS_p.12_c198_wt | SEQ ID NO: 175 | KRAS_p.12_ | SEQ ID NO: 176 | G | A |
| | 1 | | | | | c 198 mut5 | | | |
| KRAS | COSM52 | c.35G > A | p.G12D | KRAS_p.12_c209_wt | SEQ ID NO: 177 | KRAS_p.12_ | SEQ ID NO: 178 | G | A |
| | 1 | | | | | c 209 mut5 | | | |
| KRAS | COSM52 | c.35G > A | p.G12D | KRAS_p.12_c220_wt | SEQ ID NO: 179 | KRAS_p.12_ | SEQ ID NO: 180 | G | |
| | 1 | | | | | c 220 mut5 | | | |
| KRAS | COSM52 | c.35G > A | p.G12D | KRAS_p.12_c231_wt | SEQ ID NO: 181 | KRAS_p.12_ | SEQ ID NO: 182 | G | A |
| | 1 | | | | | c 231 mut5 | | | |
| KRAS | COSM52 | c.35G > A | p.G12D | KRAS_p.12_c242_wt | SEQ ID NO: 183 | KRAS_p.12_ | SEQ ID NO: 184 | G | A |
| | 1 | | | | | c 242 mut5 | | | |
| KRAS | COSM52 | c.35G > A | p.G12D | KRAS_p.12_c253_wt | SEQ ID NO: 185 | KRAS_p.12_ | SEQ ID NO: 186 | G | A |
| | 1 | | | | | c 253 mut5 | | | |
| KRAS | COSM52 | c.35G > A | p.G12D | KRAS_p.12_c264_wt | SEQ ID NO: 187 | KRAS_p.12_ | SEQ ID NO: 188 | G | A |
| | 1 | | | | | c 264mut5 | | | |
| KRAS | COSM52 | c.35G > A | p.G12D | KRAS_p.12_c275_wt | SEQ ID NO: 189 | KRAS_p.12_ | SEQ ID NO: 190 | G | A |
| | 1 | | | | | c 275_mut5 | | | |
| KRAS | COSM52 | c.35G > A | p.G12D | KRAS_p.12_c286_wt | SEQ ID NO: 191 | KRAS_p.12_ | SEQ ID NO: 192 | G | A |
| | 1 | | | | | c 286_mut5 | | | |
| KRAS | COSM52 | c.35G > A | p.G12D | KRAS_p.12_c297_wt | SEQ ID NO: 193 | KRAS_p.12_ | SEQ ID NO: 194 | G | A |
| | 1 | | | | | c 297_mut5 | | | |
| KRAS | COSM52 | c.35G > C | p.G12A | KRAS_p.12_c187_wt | SEQ ID NO: 195 | KRAS_p.12_ | SEQ ID NO: 196 | G | C |
| | 2 | | | | | c 187 mut4 | | | |
| KRAS | COSM52 | c.35G > C | p.G12A | KRAS_p.12_c198_wt | SEQ ID NO: 197 | KRAS_p.12_ | SEQ ID NO: 198 | G | C |
| | 2 | | | | | c 198 mut4 | | | |
| KRAS | COSM52 | c.35G > C | p.G12A | KRAS_p.12_c209_wt | SEQ ID NO: 199 | KRAS_p.12_ | SEQ ID NO: 200 | G | C |
| | 2 | | | | | c 209 mut4 | | | |
| KRAS | COSM52 | c.35G > C | p.G12A | KRAS_p.12_c220_wt | SEQ ID NO: 201 | KRAS_p.12_ | SEQ ID NO: 202 | G | C |
| | 2 | | | | | c220 mut4 | | | |
| KRAS | COSM52 | c.35G > C | p.G12A | KRAS_p.12_c231_wt | SEQ ID NO: 203 | KRAS_p.12_ | SEQ ID NO: 204 | G | C |
| | 2 | | | | | c231 mut4 | | | |
| KRAS | COSM52 | c.35G > C | p.G12A | KRAS_p.12_c242_wt | SEQ ID NO: 205 | KRAS_p.12_ | SEQ ID NO: 206 | G | C |
| | 2 | | | | | c242 mut4 | | | |
| KRAS | COSM52 | c.35G > C | p.G12A | KRAS_p.12_c253_wt | SEQ ID NO: 207 | KRAS_p.12_ | SEQ ID NO: 208 | G | C |
| | 2 | | | | | c253 mut4 | | | |
| KRAS | COSM52 | c.35G > C | p.G12A | KRAS_p.12_c264_wt | SEQ ID NO: 209 | KRAS_p.12_ | SEQ ID NO: 210 | G | C |
| | 2 | | | | | c264 mut4 | | | |
| KRAS | COSM52 | c.35G > C | p.G12A | KRAS_p.12_c275_wt | SEQ ID NO: 211 | KRAS_p.12_ | SEQ ID NO: 212 | G | C |
| | 2 | | | | | c275_mut4 | | | |
| KRAS | COSM52 | c.35G > C | p.G12A | KRAS_p.12_c286_wt | SEQ ID NO: 213 | KRAS_p.12_ | SEQ ID NO: 214 | G | C |
| | 2 | | | | | c286_mut4 | | | |
| KRAS | COSM52 | c.35G > C | p.G12A | KRAS_p.12_c297_wt | SEQ ID NO: 215 | KRAS_p.12_ | SEQ ID NO: 216 | G | C |
| | 2 | | | | | c297_mut4 | | | |
| KRAS | COSM51 | c.34_ | p.G12C | KRAS_p.12_c187_wt | SEQ ID NO: 217 | KRAS_p.12_ | SEQ ID NO: 218 | GGT | TGC |
| | 3 | 36GGT > TGC | | | | c187 mut2 | | | |
| KRAS | COSM51 | c.34_ | p.G12C | KRAS_p.12_c198_wt | SEQ ID NO: 219 | KRAS_p.12_ | SEQ ID NO: 220 | GGT | TGC |
| | 3 | 36GGT > TGC | | | | c198 mut2 | | | |
| KRAS | COSM51 | c.34_ | p.G12C | KRAS_p.12_c209_wt | SEQ ID NO: 221 | KRAS_p.12_ | SEQ ID NO: 222 | GGT | TGC |
| | 3 | 36GGT > TGC | | | | c209 mut2 | | | |
| KRAS | COSM51 | c.34_ | p.G12C | KRAS_p.12_c220_wt | SEQ ID NO: 223 | KRAS_p.12_ | SEQ ID NO: 224 | GGT | TGC |
| | 3 | 36GGT > TGC | | | | c220 mut2 | | | |
| KRAS | COSM51 | c.34_ | p.G12C | KRAS_p.12_c231_wt | SEQ ID NO: 225 | KRAS_p.12_ | SEQ ID NO: 226 | GGT | TGC |
| | 3 | 36GGT > TGC | | | | c231 mut2 | | | |
| KRAS | COSM51 | c.34_ | p.G12C | KRAS p.12_c242_wt | SEQ ID NO: 227 | KRAS_p.12_ | SEQ ID NO: 228 | GGT | TGC |
| | 3 | 36GGT > TGC | | | | c242 mut2 | | | |
| KRAS | COSM51 | c.34_ | p.G12C | KRAS_p.12_c253_wt | SEQ ID NO: 229 | KRAS_p.12_ | SEQ ID NO: 230 | GGT | TGC |
| | 3 | 36GGT > TGC | | | | c253 mut2 | | | |
| KRAS | COSM51 | c.34_ | p.G12C | KRAS_p.12_c264_wt | SEQ ID NO: 231 | KRAS_p.12_ | SEQ ID NO: 232 | GGT | TGC |
| | 3 | 36GGT > TGC | | | | c264 mut2 | | | |
| KRAS | COSM51 | c.34_ | p.G12C | KRAS_p.12_c275_wt | SEQ ID NO: 233 | KRAS_p.12_ | SEQ ID NO: 234 | GGT | TGC |
| | 3 | 36GGT > TGC | | | | c275_mut2 | | | |
| KRAS | COSM51 | c.34_ | p.G12C | KRAS_p.12_c286_wt | SEQ ID NO: 235 | KRAS_p.12_ | SEQ ID NO: 236 | GGT | TGC |
| | 3 | 36GGT > TGC | | | | c286_mut2 | | | |
| KRAS | COSM51 | c.34_ | p.G12C | KRAS_p.12_c297_wt | SEQ ID NO: 237 | KRAS_p.12_ | SEQ ID NO: 238 | GGT | TGC |
| | 3 | 36GGT > TGC | | | | c297_mut2 | | | |
| KRAS | COSM14 | c.35_ | p.G12D | KRAS_p.12_c187_wt | SEQ ID NO: 239 | KRAS_p.12_ | SEQ ID NO: 240 | GT | AC |
| | 209 | 36GT > AC | | | | c187 mut3 | | | |
| KRAS | COSM14 | c.35_ | p.G12D | KRAS_p.12_c198_wt | SEQ ID NO: 241 | KRAS_p.12_ | SEQ ID NO: 242 | GT | AC |
| | 209 | 36GT > AC | | | | c198 mut3 | | | |
| KRAS | COSM14 | c.35_ | p.G12D | KRAS_p.12_c209_wt | SEQ ID NO: 243 | KRAS_p.12_ | SEQ ID NO: 244 | GT | AC |
| | 209 | 36GT > AC | | | | c209_mut3 | | | |
| KRAS | COSM14 | c.35_ | p.G12D | KRAS_p.12_c220_wt | SEQ ID NO: 245 | KRAS_p.12_ | SEQ ID NO: 246 | GT | AC |
| | 209 | 36GT > AC | | | | c220 mut3 | | | |
| KRAS | COSM14 | c.35_ | p.G12D | KRAS_p.12_c231_wt | SEQ ID NO: 247 | KRAS_p.12_ | SEQ ID NO: 248 | GT | AC |
| | 209 | 36GT > AC | | | | c231 mut3 | | | |
| KRAS | COSM14 | c.35_ | p.G12D | KRAS_p.12_c242_wt | SEQ ID NO: 249 | KRAS_p.12_ | SEQ ID NO: 250 | GT | AC |
| | 209 | 36GT > AC | | | | c242 mut3 | | | |
| KRAS | COSM14 | c.35_ | p.G12D | KRAS_p.12_c253_wt | SEQ ID NO: 251 | KRAS_p.12_ | SEQ ID NO: 252 | GT | AC |
| | 209 | 36GT > AC | | | | c253_mut3 | | | |
| KRAS | COSM14 | c.35_ | p.G12D | KRAS_p.12_c264_wt | SEQ ID NO: 253 | KRAS_p.12_ | SEQ ID NO: 254 | GT | AC |
| | 209 | 36GT > AC | | | | c264 mut3 | | | |
| KRAS | COSM14 | c.35_ | p.G12D | KRAS_p.12_c275_wt | SEQ ID NO: 255 | KRAS_p.12_ | SEQ ID NO: 256 | GT | AC |
| | 209 | 36GT > AC | | | | c275_mut3 | | | |
| KRAS | COSM14 | c.35_ | p.G12D | KRAS_p.12_c286_wt | SEQ ID NO: 257 | KRAS_p.12_ | SEQ ID NO: 258 | GT | AC |
| | 209 | 36GT > AC | | | | c286_mut3 | | | |
| KRAS | COSM14 | c.35_ | p.G12D | KRAS_p.12_c297_wt | SEQ ID NO: 259 | KRAS_p.12_ | SEQ ID NO: 260 | GT | AC |
| | 209 | 36GT > AC | | | | c297_mut3 | | | |
| KRAS | COSM51 | c.34G > T | p.G12C | KRAS_p.12_c187_wt | SEQ ID NO: 261 | KRAS_p.12_ | SEQ ID NO: 262 | G | T |
| | 6 | | | | | c187 mut1 | | | |
| KRAS | COSM51 | c.34G > T | p.G12C | KRAS_p.12_c198_wt | SEQ ID NO: 263 | KRAS_p.12_ | SEQ ID NO: 264 | G | T |
| | 6 | | | | | c198 mut1 | | | |
| KRAS | COSM51 | c.34G > T | p.G12C | KRAS_p.12_c209_wt | SEQ ID NO: 265 | KRAS_p.12_ | SEQ ID NO: 266 | G | T |
| | 6 | | | | | c209 mut1 | | | |
| KRAS | COSM51 | c.34G > T | p.G12C | KRAS_p.12_c220_wt | SEQ ID NO: 267 | KRAS_p.12_ | SEQ ID NO: 268 | G | T |
| | 6 | | | | | c220 mut1 | | | |
| KRAS | COSM51 | c.34G > T | p.G12C | KRAS p.12_c231_wt | SEQ ID NO: 269 | KRAS_p.12_ | SEQ ID NO: 270 | G | T |
| | 6 | | | | | c231 mut1 | | | |
| KRAS | COSM51 | c.34G > T | p.G12C | KRAS_p.12_c242_wt | SEQ ID NO: 271 | KRAS_p.12_ | SEQ ID NO: 272 | G | T |
| | 6 | | | | | c242 mut1 | | | |
| KRAS | COSM51 | c.34G > T | p.G12C | KRAS_p.12_c253_wt | SEQ ID NO: 273 | KRAS_p.12_ | SEQ ID NO: 274 | G | T |
| | 6 | | | | | c253 mut1 | | | |
| KRAS | COSM51 | c.34G > T | p.G12C | KRAS_p.12_c264_wt | SEQ ID NO: 275 | KRAS_p.12_ | SEQ ID NO: 276 | G | T |
| | 6 | | | | | c264 mut1 | | | |
| KRAS | COSM51 | c.34G > T | p.G12C | KRAS_p.12_c275_wt | SEQ ID NO: 277 | KRAS_p.12_ | SEQ ID NO: 278 | G | T |
| | 6 | | | | | c275 mut1 | | | |
| KRAS | COSM51 | c.34G > T | p.G12C | KRAS_p.12_c286_wt | SEQ ID NO: 279 | KRAS_p.12_ | SEQ ID NO: 280 | G | T |
| | 6 | | | | | c286_mut1 | | | |
| KRAS | COSM51 | c.34G > T | p.G12C | KRAS_p.12_c297_wt | SEQ ID NO: 281 | KRAS_p.12_ | SEQ ID NO: 282 | G | T |
| | 6 | | | | | c297 mut1 | | | |

### Example 3: Detection of Alleles by Contacting a Substrate Bound to an Enriched Nucleic Acid Sample with Locus-Specific Probes and Allele-Specific Probes

Fragmented genomic DNA prepared as described above in Example 1 and then are bound and distributed onto the surface of an epoxy-coated silicon substrate as described above in Example 2. Locus-specific probes comprising fluorescent tags, each tag corresponding to a particular locus are contacted with the substrate and the locus-specific probes are allowed to hybridize to the genomic locus of interest under high or low stringency conditions. The array surface is then washed under high or low stringency wash conditions to remove unbound locus-specific probes. The fluorescence is detected using an optical imaging system to detect the presence of the locus at individual locations on the array. Allele-specific probes comprising fluorescent-tags are contacted with array with M=10 cycles as described above in Example 2. Analysis of color codes for identification of sequences is performed using a two-color imaging system. Mapping of target identification sequence to color sequence is performed such that each color corresponds to a sequence, which maps to 1 or 0 with 1 bit of information being acquired per cycle. The error correction scheme is conservative and requires zero errors per target, an error is defined as a positive identification in a sequence where it is not expected. Up to five missing sequences are allowed per molecule. Missing sequences are cases where a molecule is not identified in a cycle and are not classified as errors.

### Example 4: Detection of Epidermal Growth Factor Receptor (EGFR) Exon 19 Deletion Mutations Using Allele-Specific Probes

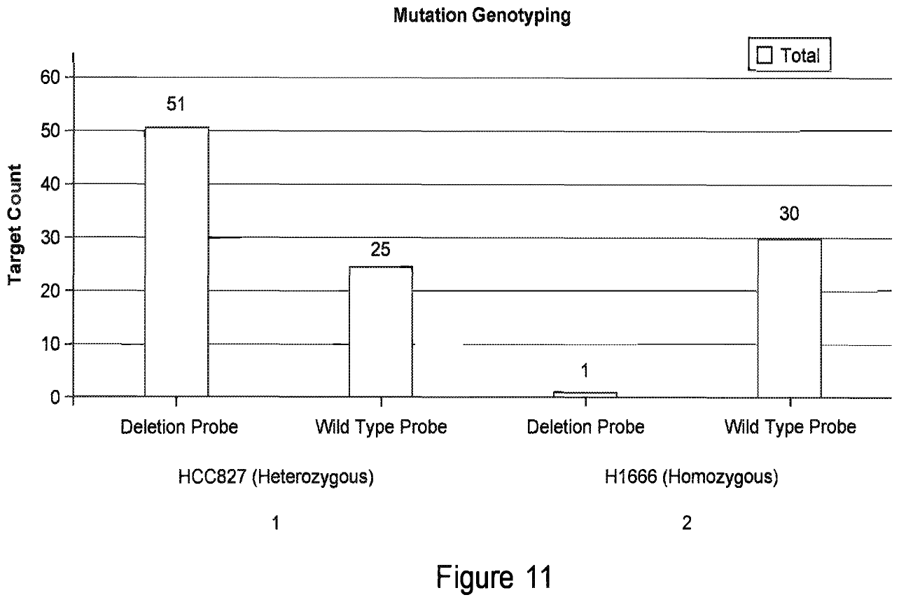

Detection for EGFR deletion mutation (E747 A750) on exon 19 was performed by hybridization of allele-specific probes to enriched genomic DNA isolated from two cell lines: the Non-Small Cell Lung Cancer (NSCLC) cell line, HCC827, heterozygous for the E746-A750 deletion mutation and the lung adenocarcinoma cell line, H1666, homozygous for the wild-type EGFR gene. Enriched genomic DNA samples were loaded on carbohydrazide activated slides using EDC chemistry. Ten cycles comprising hybridization, washing and stripping of probes were performed. Two allele-specific probes were used, one probe specific to the wild-type allele and another probe specific for the E747 A750 deletion mutation. The assay resulted in efficient detection of mutant and the wild type alleles in the heterozygous HCC827 cell line; while the probe did not detect the deletion mutation in the wild-type H1666 cell line (FIG. 11).

### Example 5: Detection of Single Nucleotide Polymorphisms Using a Single Base Extension Reaction

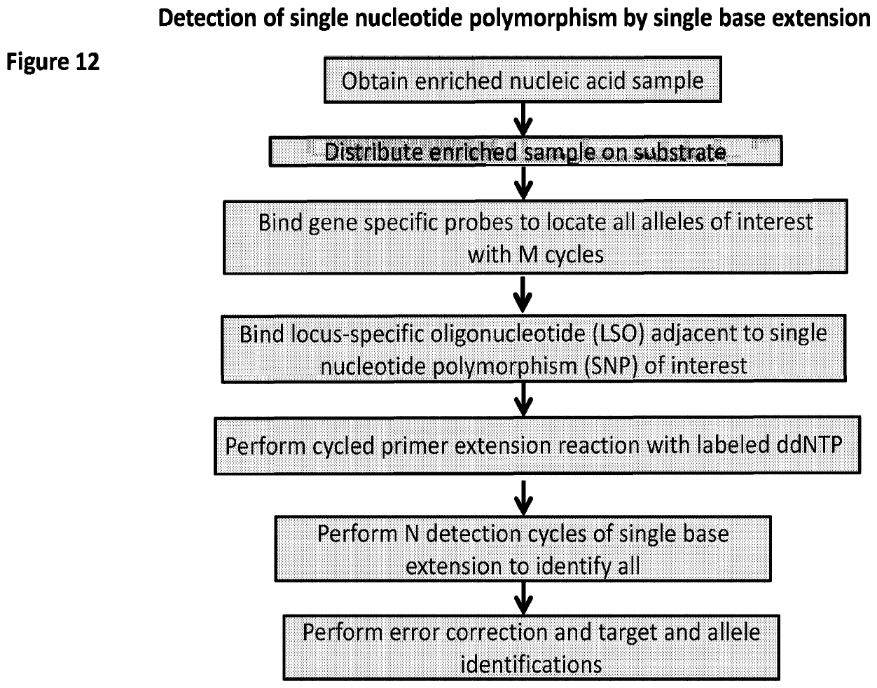

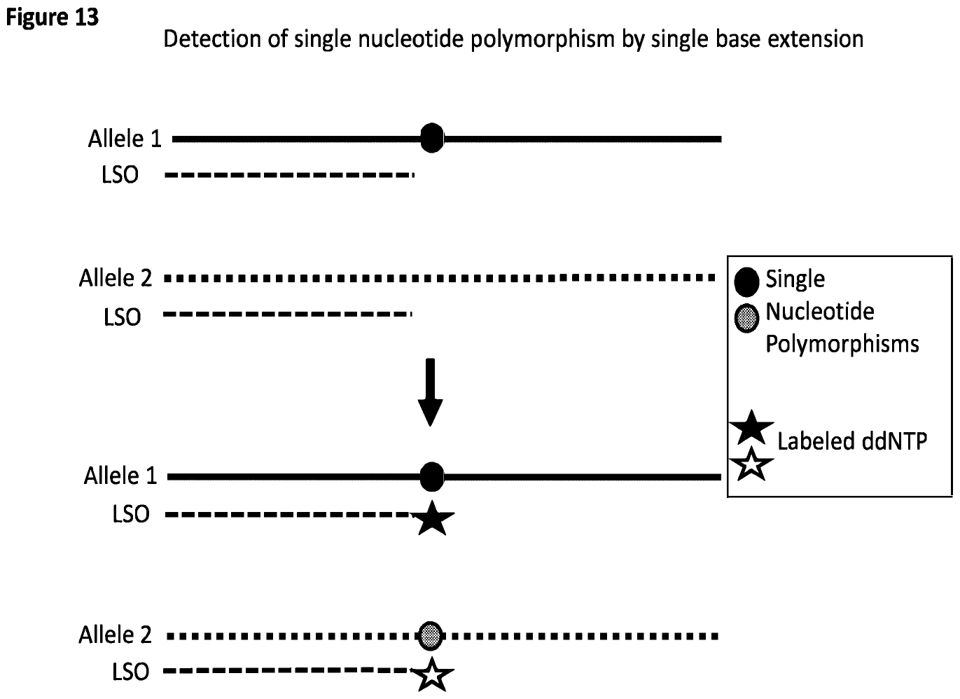

Fragmented genomic DNA prepared as described above in Example 1 and then fragmented single stranded genomic DNA fragments are bound and distributed onto the surface of an epoxy-coated silicon substrate as described above in Example 2. The genomic DNA is then subjected to M=10 detection cycles wherein each detection cycle comprises a single nucleotide base extension (SBE) reaction (FIG. 12 and FIG. 13). To perform the SBE reaction, unlabeled oligonucleotide primers complementary to loci of interest are annealed with the genomic ssDNA at 42° C. for 5 minutes. Examples of oligonucleotide primers for detection of mutations in BRAF and EGFR genes are shown in Table 3 below. Extension is performed for 30 seconds at 72° C. to allow polymerase to extend the primer using fluorescently labeled ddNTPs comprising (ddATP, ddTTP, ddCTP and ddGTP) wherein each of the 4 ddNTPs are labeled with a unique fluorescent tag. The array is then washed under high or low stringency conditions to remove the unincorporated ddNTPs. The fluorescence on the extended primers at each region on the array is then detected using an optical imaging system (GenePix® 4200A microarray scanner provided by Axon Instruments™). UM is less than 10, the primers are then denatured from the array and genomic ssDNA fragments in preparation for the subsequent detection cycle. Analysis of color codes for identification of sequences is performed using a two-color imaging system. Mapping of target identification sequence to color sequence is performed such that each color corresponds to a sequence, which maps to 1 or 0 with 1 bit of information being acquired per cycle. The error correction scheme is conservative and requires zero errors per target, an error is defined as a positive identification in a sequence where it is not expected. Up to five missing sequences are allowed per molecule. Missing sequences are cases where a molecule is not identified in a cycle and are not classified as errors.

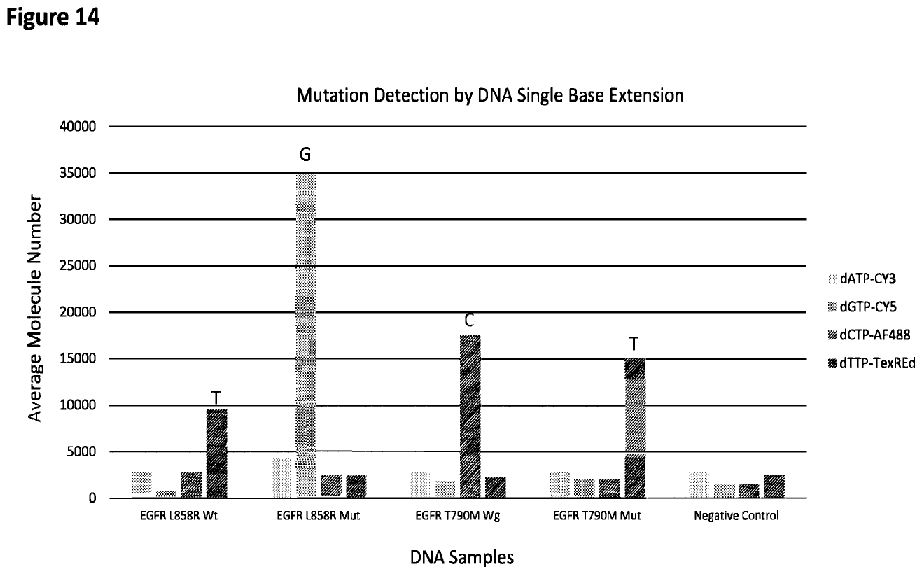

Wild type and mutant DNA targets for EGFR L858M and EGFR T790M were loaded on the surface of different flow cells. Oligonucleotide primers complementary to the target and with 3′ terminal adjacent to the nucleotide base to be identified were first annealed to the DNA targets. The oligonucleotide primer was then enzymatically extended by single base in the presence of four dye labeled nucleotides with a 3′ blocker (dCTP-AF488, dATP-AFCy3, dTTP-TexRed, and dGTP-Cy5). The nucleotide complementary to the base in the DNA template was incorporated and then identified (FIG. 14). These results confirm the detection of single nucleotide mutations in the EGFR gene by the single base extension methods described herein.

TABLE 3
Probes for Detection Using a Single Base Extension Reaction

| Gene | COSMIC ID | CDS Mutation | AA Mutation | Probe Sequence |
|---|---|---|---|---|
| BRAF | COSM476 | c.1799T > A | p.V600E | SEQ ID NO: 283 |
| | | (Substitution, | (Substitution- | |
| | | position 1799, | Missense, | |
| | | T → A) | position 600, | |
| | | | V → E) | |
| EGFR | COSM6224 | c.2573T > G | p.L858R | SEQ ID NO: 284 |
| | | (Substitution | (Substitution | |
| | | position 2573, | Missense, | |
| | | T → G) | position 858, | |
| | | | L → R) | |
| EGFR | COSM6240 | c.2369C > T | p.T790M | SEQ ID NO: 285 |
| | | (Substitution | (Substitution- | |
| | | position 2369, | Missense, | |
| | | C → PT) | position 790, | |
| | | | T → M) | |

### Example 6: Detection of Alleles of Interest by Detection of Amplification Products

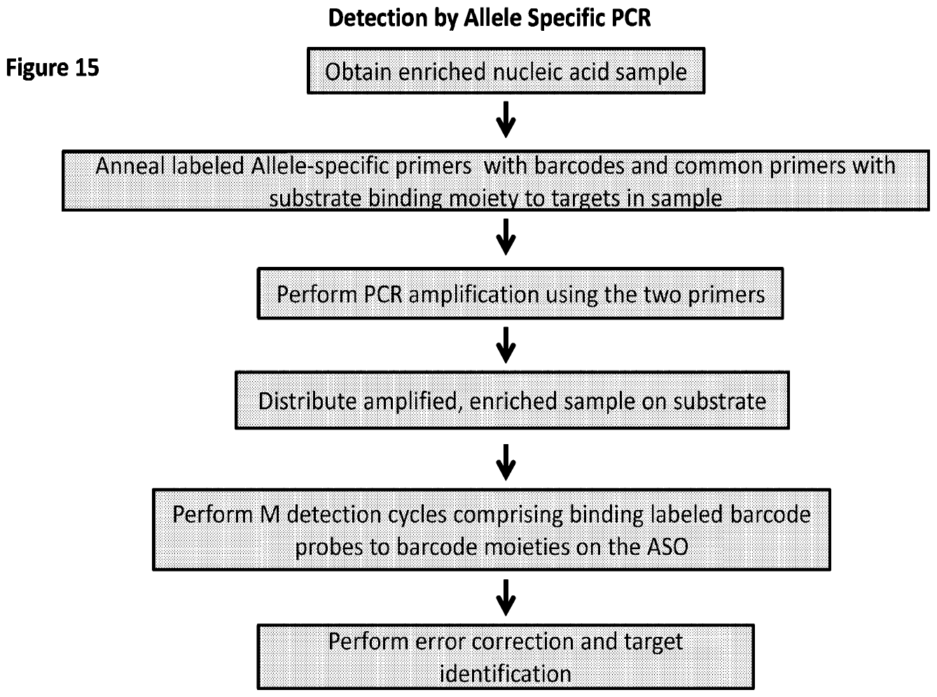

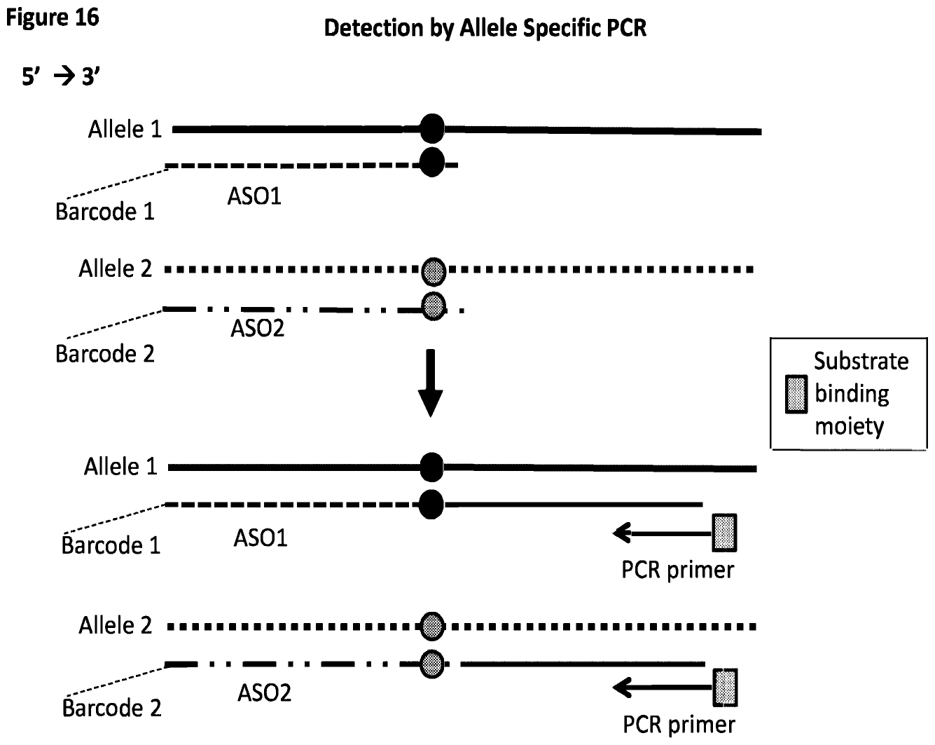

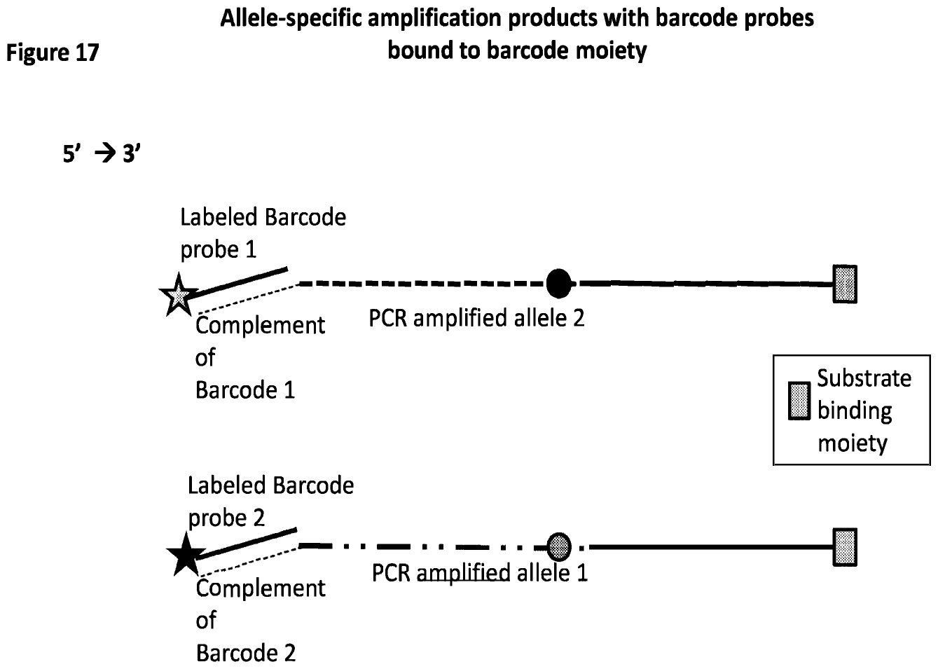

Fragmented genomic DNA prepared as described above in Example 1. Allele-specific PCR is then performed on the fragmented, enriched nucleic acid sample as described in FIGS. 15-17. Allele specific amplification reactions (AS-PCR) are performed on the fragmented genomic DNA. 200 ng of genomic DNA and a master mix based on the Expand High Fidelity Polymerase kit (no. 11759078001; Roche, Indianapolis, IN) with 1.4 U of polymerase, 160 mol/L dNTP (Stratagene, Cedar Creek, TX), 400 nmol/L nucleotide sequence variant-specific primers or allele-specific primers bound to a barcode moiety and 800 nmol/L reverse locus-specific primer bound to biotin. Examples of allele-specific primers are shown in Table 4 below. The cycling conditions for the amplification reaction are as follows: 95° C. for 1 minute, followed by 45 cycles of 94° C. for 1 minute, 55° C. for 1 minute and 72° C. for 1 minute, and a final 7-minute incubation at 73° C. The amplification products derived from the fragmented single stranded genomic DNA fragments are denatured to produce single stranded DNA and then are bound and distributed onto the surface of a streptavidin-coated glass surface in an array format, as described in Example 1. M=10 detection cycles are performed, wherein each detection cycle comprises contacting the array with barcode probes (FIG. 15 and FIG. 17). In each detection cycle, barcode probes comprising fluorescently-labeled tags are complementary to the barcode moieties are hybridized to the amplification products under high or low stringency conditions, the array surface is then washed to remove unhybridized barcode probes, and the fluorescence at each region on the array is detected using an optical imaging system (GenePix® 4200A microarray scanner provided by Axon Instruments™). If M is less than 10, the barcode probes annealed to the barcode moieties are denatured and the surface of the array is washed to remove the barcode probes in preparation for the subsequent detection cycle. Analysis of color codes for identification of sequences is performed using a two-color imaging system. Mapping of target identification sequence to color sequence is performed such that each color corresponds to a sequence, which maps to 1 or 0 with 1 bit of information being acquired per cycle. The error correction scheme is conservative and requires zero errors per target, an error is defined as a positive identification in a sequence where it is not expected. Up to five missing sequences are allowed per molecule. Missing sequences are cases where a molecule is not identified in a cycle and are not classified as errors.

### Table 4: Probes for Detection Using Allele-Specific Amplification

TABLE 4
Probes for Detection Using Allele-Specific Amplification

| | | | | Forward | | | | |
|---|---|---|---|---|---|---|---|---|
| | | | | Primer | Forward | | | |
| | COSMIC | CDS | | Wild | Primer | Reverse | Wild | |
| Gene | ID | Mutation | AA Mutation | Type | Mutant | Primer | Type | Mutation |
| BRAF | COSM476 | c. 1799T > A | p. V600E | SEQ ID | SEQ ID | SEQ ID | T | A |
| | | (Substitution, | (Substitution- | NO: 286 | NO: 287 | NO: 288 | | |
| | | position 1799, | Missense, | | | | | |
| | | T→A) | position 600, | | | | | |
| | | | V→E) | | | | | |
| EGFR | COSM6224 | c. 2573T > G | p. L858R | SEQ ID | SEQ ID | SEQ ID | T | G |
| | | (Substitution, | (Substitution- | NO: 289 | NO: 290 | NO: 291 | | |
| | | position 2573, | Missense, | | | | | |
| | | T→G) | position 858, | | | | | |
| | | | L→PR) | | | | | |
| EGFR | COSM6240 | c. 2369C > T | p. T790M | SEQ ID | . SEQ ID | SEQ ID | C | T |
| | | (Substitution, | (Substitution- | NO: 292 | NO: 293 | NO: 294 | | |
| | | position 2369, | Missense, | | | | | |
| | | C→T) | position 790, | | | | | |
| | | | T→M) | | | | | |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1.-193. (canceled)

194. A method of detecting at least one target nucleotide sequence variant suspected of being present in a sample, comprising:
(a) providing a ligation reaction product of a target-dependent oligonucleotide ligation reaction performed on said sample, wherein said ligation reaction product comprises a plurality of oligonucleotides each comprising a substrate binding moiety and a barcode moiety;
(b) distributing said ligation reaction product on a substrate such that individual oligonucleotides bind to the substrate via said substrate binding moiety at spatially separate regions of said substrate;
(c) carrying out on said substrate a target nucleotide sequence variant identification assay, wherein said sequence variant identification assay comprises performing at least M detection cycles to generate a signal detection sequence, wherein M is at least two, each cycle comprising:
(i) contacting said ligation reaction product with a barcode probe comprising a detection label, wherein said barcode probe binds to a barcode moiety when it is present on said substrate;
(ii) washing a surface of said substrate to remove unbound barcode probes;
(iii) detecting an identity and location of said detection label on said substrate; and
(iv) if a cycle number is less than said M detection cycles, removing said barcode probe from said barcode moiety; and
(d) analyzing a signal detection sequence generated by said M cycles at spatially separate locations on said substrate to determine a presence or absence of said at least one target nucleotide sequence variant.

195. The method of claim 194, wherein said ligation reaction product comprises an oligonucleotide comprising a sequence variant-specific oligonucleotide sequence, a locus-specific oligonucleotide sequence, a binding moiety, and a barcode moiety.

196. The method of claim 194, wherein providing said ligation reaction product comprises carrying out said target-dependent oligonucleotide ligation reaction on said sample suspected of comprising at least one target nucleotide sequence variant.

197. The method of claim 194, wherein said sample is an enriched nucleic acid sample suspected of comprising at least one target nucleotide sequence variant of a plurality of sequence variants at one of a plurality of target loci.

198. The method of claim 197, wherein said enriched nucleic acid sample is enriched by performing a reverse transcription reaction on a sample comprising RNA.

199. The method of claim 194, wherein said barcode probe comprises a unique label between at least two different cycles.

200. The method of claim 194, wherein analyzing said signal detection sequence comprises comparing said signal detection sequence with an anticipated signal detection sequence for said target nucleotide sequence variant and determining a probability score for a presence or absence of said target nucleotide sequence variant of interest based on said signal detection sequence.

201. The method of claim 200, wherein said comparing reduces an error due to misidentification of said target in at least one of said M cycles.

202. The method of claim 201, wherein said misidentification event is due to a false positive or a false negative signal.

203. The method of claim 194, wherein said at least one target nucleotide sequence variant is an allele.

204. A method for determining that at least one sequence variant is present at a locus of a nucleic acid molecule, comprising:
(a) providing an array comprising a plurality of distinct nucleic acid molecules, wherein said plurality of distinct nucleic acid molecules comprises said nucleic acid molecule, wherein said nucleic acid molecule comprises a sequence having an allele-specific nucleic acid molecule and a locus-specific nucleic acid molecule hybridized thereto, and wherein said allele-specific nucleic acid molecule and said locus-specific nucleic acid molecule are ligated to one another;
(b) subjecting said nucleic acid molecule or derivative thereof to a sequence identification to yield at least a first signal and at least a second signal, wherein said first signal is specific to said allele-specific nucleic acid molecule and said second signal is specific to said locus-specific nucleic acid molecule; and
(c) using at least said first signal and at least said second signal to determine that said at least one sequence variant is present at said locus of said nucleic acid molecule.

205. The method of claim 204, wherein said sequence identification comprises sequencing.

206. The method of claim 204, wherein said allele-specific nucleic acid molecule and said locus-specific nucleic acid molecule comprise a detectable label, and wherein said detectable label generates said first signal or said second signal.

207. The method of claim 206, wherein said sequence identification comprises detecting said detectable label.

208. The method of claim 204, further comprising, prior to (a), providing a nucleic acid sample, and performing a reverse transcription reaction on a ribonucleic acid (RNA) molecule from said nucleic acid sample to yield said nucleic acid molecule.

209. The method of claim 208, wherein said nucleic acid sample is derived from a plurality of origins.

210. The method of claim 204, further comprising, prior to (b), separating said allele-specific nucleic acid molecule and said locus-specific nucleic acid molecule from said sequence of said nucleic acid molecule.

211. The method of claim 204, wherein said locus comprises at least a portion of a gene.

212. The method of claim 204, wherein said at least one sequence variant comprises a deletion, a replacement, a rearrangement, or an insertion.

213. The method of claim 204, wherein said at least one sequence variant comprises a single nucleotide polymorphism.
